(19) United States
(12) Patent Application Publication
(10) Pub. No.: US 2023/0241105 A1
(43) Pub. Date: Aug. 3, 2023

(54) METHODS OF USE OF ALLERGEN-SPECIFIC T CELLS IN ALLERGY AND ASTHMA

(71) Applicants: Gregory SEUMOIS, La Jolla, CA (US); La Jolla Institute for Immunology, La Jolla, CA (US)

(72) Inventors: Gregory Seumois, La Jolla, CA (US); Pandurangan Vijayanand, La Jolla, CA (US)

(73) Assignee: La Jolla Institute for Immunology, La Jolla, CA (US)

(21) Appl. No.: 18/008,633

(22) PCT Filed: Jun. 10, 2021
(86) PCT No.: PCT/US2021/036882

§ 371 Date: Dec. 6, 2022

Related U.S. Application Data
(60) Provisional application No. 63/038,057, filed on Jun. 11, 2020.

(51) Int. Cl. A61K 35/17; C12N 5/0783; C07K 14/705; C12Q 1/6881; A61K 45/06; A61P 11/06; A61P 37/08

(52) U.S. Cl. CPC A61K 35/17; C12N 5/0637; C07K 14/70575; C12Q 1/6881; A61K 45/06; A61P 11/06; A61P 37/08; C12Q 2600/158

(57) ABSTRACT

Disclosed herein are methods of detecting an antigen-specific T cell (e.g., a HDM-reactive T cell) expressing IL-9 which contribute to a worsening of an allergy (e.g., a HDM-induced allergy) or detecting an antigen-specific T cell (e.g., a HDM-reactive T cell) associated with a positive prognosis. In some embodiments, also disclosed herein is a method of treating an allergy (e.g., a HDM-induced allergy) in a subject in need thereof based on the presence or absence of an antigen-specific T cell (e.g., a HDM-reactive T cell) that express IL-9.

### CROSS-REFERENCE TO RELATED APPLICATIONS

This application claims priority under 35 U.S.C. § 119(e) of U.S. Provisional Application No. 63/038,057, filed Jun. 11, 2020, the contents of which are incorporated by reference in its entireties into the present application.

### STATEMENT AS TO FEDERALLY SPONSORED RESEARCH

This invention was made with United States government support under federal grants U19AI100275, U19AI135731, and R01HL114093 awarded by the National Institute of Health. The United States government has certain rights in the invention.

### BACKGROUND

House dust mites (HDM; Dermatophagoides spp.) are one the most common source of air-bone allergen. HDMs are members of the subclass Acari and HDMs isolated from dust samples comprise predominately species Dermatophagoides pteronyssinus and Dermatophagoides farina. CD4+ helper T cells (T_{H}) and regulatory T cells (T_{reg}) that respond to common allergens play a role in driving and dampening airway inflammation in patients with asthma. Crosstalk between the innate and adaptive immune system further modulates the initiation and propagation of the allergic Th2 response. As such, there is a need to further understanding the diverse determinants that contribute to HDM allergenicity. This disclosure satisfies this need and provides related advantages as well.

### SUMMARY

Disclosed herein, in certain embodiments, are methods of detecting an antigen-specific T cell (e.g., a HDM-reactive T cell) that contribute to a worsening of an allergy (e.g., a HDM-induced allergy) or associated with a positive prognosis. In some embodiments, disclosed herein is a method of detecting the presence of an antigen-specific T cell (e.g., a house dust mite (HDM)-reactive T cell) characterized with an elevated expression level of an interferon response gene profile in a biological sample, comprising, or consisting essentially of, or yet further consisting of: (a) generating a gene expression profile of at least one T cell from each of a population of CD154-enriched T cells, a population of CD137-enriched T cells, or a population of CD4-enriched T cells obtained from a biological sample by a sequencing method; and (b) analyzing the gene expression profile to detect the presence of the antigen-specific T cell (e.g., a HDM-reactive T cell) that expresses the elevated level of the interferon response gene profile in the biological sample.

In some embodiments, also disclosed herein is a method of detecting the presence of an interleukin 9 (IL-9)-expressing antigen-specific T cell (e.g., an IL-9-expressing HDM-reactive T cell) in a biological sample, comprising, or consisting essentially of, or yet further consisting of: (a) generating a gene expression profile for at least one T cell from each of a population of CD154-enriched T cells, a population of CD137-enriched T cells, or a population of CD4-enriched T cells obtained from a biological sample by a sequencing method; and (b) analyzing the gene expression profile to detect the presence of the IL-9-expressing antigen-specific T cell (e.g., an IL-9-expressing HDM-reactive T cell) in the biological sample.

In some embodiments, further disclosed herein is a method of treating an allergy (e.g., a house dust mite (HDM)-induced allergy) in a subject in need thereof or selecting a subject for treatment of an allergy (e.g., a HDM-induced allergy), comprising, or consisting essentially of, or yet further consisting of: detecting the presence of an IL-9-expressing antigen-specific T cell (e.g., an IL-9-expressing HDM-reactive T cell) in a biological sample obtained from the subject to identify the subject has expressing the IL-9-expressing antigen-specific T cell (e.g., the IL-9-expressing HDM-reactive T cell); and administering a therapeutic agent selected from an antihistamine, a corticosteroid, a mast cell stabilizer, and a leukotriene modifier to the subject expressing the IL-9-expressing antigen-specific T cell (e.g., the IL-9-expressing HDM-reactive T cell).

In some embodiments, additionally disclosed herein is a composition comprising, or consisting essentially of, or yet further consisting of a plurality of antigen-specific T cells (e.g., HDM-reactive T cells), detectable using the method of detecting as disclosed herein and subsequently isolating an antigen-specific T cell (e.g., a house dust mite (HDM)-reactive T cell), characterized with an elevated expression level of an interferon response gene profile in a biological sample; and optionally comprising a carrier, excipient, stabilizer or preservative. In some embodiments, disclosed herein is a method of treating an allergy (e.g., a house dust mite (HDM)-induced allergy) in a subject in need thereof comprising administering to the subject the composition.

In some embodiments, further disclosed herein is a method of generating a TRAIL-expressing T cell, comprising, or consisting essentially of, or yet further consisting of: incubating a biological sample comprising a plurality of CD4+ T cells with an isolated and recombinant TRAIL protein for at least 10 minutes to generate a population of stimulated T cells; and separating the population of stimulated T cells by a flow cytometry method to isolate a TRAIL-expressing T cell.

In some embodiments, provided is a method of generating a TRAIL-expressing T cell. The method comprises, or alternatively consists essentially of, or yet further consists of incubating a biological sample comprising a plurality of CD4+ T cells with a TCR stimulator, optionally selected from an antigen, an anti-CD3 antibody, an anti-CD28 antibody, or any combination thereof, for at least 10 minutes to generate a population of stimulated T cells; and isolating or enriching or both isolating and enriching TRAIL-expressing T cells from the stimulated T cells. In some embodiments, the isolated or enriched T cells comprise an elevated expression level of an interferon response gene profile as disclosed herein.

In some embodiments, provided is a method comprising, or alternatively consisting essentially of, or yet further consisting of expanding or isolating or both expanding and isolating a plurality of CD4+ T cells that express an elevated level of an interferon response gene profile. In further embodiments, the method further comprises stimulating the plurality of CD4+ T cells by contacting with a TCR stimulator, optionally selected from an anti-CD3 antibody or an anti-CD28 antibody or both. In yet further embodiments, the isolated or enriched T cells comprise a TRAIL-expressing T_{H} cell or a TRAIL-expressing T_{REG} cell.

In further embodiments, provided are T cells isolated or enriched using a method as disclosed herein. In further embodiments, provided is a composition comprising the isolated or enriched T cells and optionally a carrier, excipient, stabilizer or preservative. In some embodiments, the T cells isolated or enriched or detected using a method as disclosed herein are engineered to express a chimeric antigen receptor (CAR). Additionally or alternatively, the T cells isolated or enriched or detected using a method as disclosed herein express a T cell receptor (TCR). In some embodiments, the CAR or TCR or both specifically recognize and bind to an antigen of a pathogen or a cancer. Accordingly, the composition can be used to treat a subject having or suspect of having an infection caused by the pathogen or the cancer.

In yet further embodiments, provided are methods of one or more of the following: (i) treating an allergy in a subject in need thereof, (ii) treating an infection in a subject in need thereof, or (iii) treating an inflammation in a subject in need thereof. The method comprises, or alternatively consists essentially of, or yet further consists of administering to the subject, for example a therapeutically effective amount of, the T cells isolated or enriched or detected using a method as disclosed herein. In some embodiments, the method comprises, or alternatively consists essentially of, or yet further consists of administering a composition as disclosed herein to the subject.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1D illustrate bulk RNA-seq analysis of HDM allergen-reactive T cells does not identify asthma-specific features. (FIG. 1A) Dot plot showing the percentage of CD4 memory HDM-reactive T cells that were CD154⁺ (left) or CD154⁻CD137⁺ (right) for each subject group. Horizontal line mean; error bar, standard error. (FIG. 1B) t-SNE plot of bulk RNA-seq samples: 6 HDM⁻ T cells (from asthma-allergic patients, 24 HDM⁺ T_{H} (from all patients), 18 HDM⁺ T_{reg} (from all patients)). (FIG. 1C) Gene set enrichment analysis (GSEA) of grouped bulk RNA-seq datasets presented in FIG. 1B. q, false discovery rate; NES, normalized enrichment score; RES, relative enrichment score. (FIG. 1D) Scatter plot shows log₂-fold change in expression of significantly differentially expressed genes between asthma (N=24) versus non-asthma (N=24) (x axis) and allergic (N=24) versus non-allergic (N=24) (y axis) T_{H} samples.

FIGS. 2A-2B illustrate single-cell RNA-seq clustering analysis reveals heterogeneity among HDM-reactive T_{H} cells. (FIG. 2A) Heatmap of row-wise z-score-normalized mean expression of cluster-specific differentially expressed genes. Columns represent the average expression for each cluster, ordered based on biological relevance. Right—lists of biologically relevant example genes for each cluster. Equal numbers of cells were sampled from each cluster. (FIG. 2B) Violin plots show log₂ (CPM+1) normalized expression in each cluster (3 T_{H}ACT clusters merged) for 24 cluster-specific signature genes (6 per cell type). Grey scale represents the fraction of cells within each cluster expressing the given gene, excluding cells with no expression. q, false discovery rate; NES, normalized enrichment score; RES, relative enrichment score.

FIGS. 3A-3E illustrate proportions of HDM-reactive T_{H} subsets differ between allergic and non-allergic subjects. (FIG. 3A) t-SNE visualization of Seurat clustering analysis, using equal cell numbers for each disease group (N=3,720), obtained from all 6 subjects in each group. Cells are shaded/marked. (FIG. 3B) Scatter plot shows the log₂-fold change of expression of T_{H}IFNR signature genes between asthmatic (N=12) versus non-asthmatic (N=12) (x-axis) and allergic (N=12) versus non-allergic (N=12) (y-axis) subjects among cells in the T_{H}IFNR cluster. Equal numbers of cells were sampled per group. Dotted lines indicate the threshold value of fold change for gene filtering. (FIG. 3C) Scatter plots show co-expression of TNFSF10 with the products of the T_{H}INFR signature genes IFI6 and ISG15 by T_{H}INFR cells (left) or HDM⁻ T cells (right). Each dot represents one cell. (FIG. 3D) Contour plots show the expression of CD69 versus TRAIL in memory CD4⁺ T cells before (left) and after 6 h (center) of TCR stimulation with anti-CD3 and anti-CD28. Both plots are representative of 5 independent experiments. Numbers indicate the percentage of cells in each quadrant. Right, quantification of each of the 6 experiments; bars represent the mean and standard error. P<0.01 (Student t test). (FIG. 3E) Left, contour plots show the expression of the cell-activation markers CD154, CD69, and CD137 in memory CD4⁺ cells after 6 h of stimulation in the presence or absence of hrTRAIL (human recombinant TRAIL). Data shown are from a representative experiment. Right, quantification of each of the 6 experiments; bars represent the mean and standard error. *, P<0.1; **, P<0.01, *** P<0.001 (Student t test).

FIGS. 4A-4E illustrate a subset of HDM-reactive T_{reg} cells express the interferon response signature. (FIG. 4A) t-SNE visualization of Seurat clustering analysis of the transcriptomes of 10,526 single HDM-activated T_{reg} cells obtained from all 24 subjects. (FIG. 4B) Heatmap showing hierarchical clustering of row-wise z-score-normalized mean expression of cluster-specific differentially expressed genes (N=1,559) Columns represent each T_{reg} cluster. Right—lists of biologically relevant examples genes for the T_{reg} IFNR cluster. Equal numbers of cells were sampled per disease group. (FIG. 4C) t-SNE visualization of Seurat clustering analysis shown in FIG. 4A, using equal cell numbers for each subject group (n=2,180) obtained from all 6 subjects. (FIG. 4D) GSEA between each T_{reg}IFNR and the 2 other T_{reg} clusters for the Type I and II IFN signaling list of genes. q, false discovery rate; NES, normalized enrichment score; RES, relative enrichment score. (FIG. 4E) Violin plots show log₂(CPM+1) normalized expression of TNFSF10 in each T_{reg} cluster. Cells with no expression are excluded (see Materials and Methods).

FIG. 5 illustrates HDM-reactive T_{H}2 cells are enriched for transcripts linked to enhanced functionality. Hierarchical clustering of Spearman correlation coefficient matrix for saver-imputed expression values of the 214 genes uniquely up-regulated in the T_{H}2 cluster. Values are clustered with complete linkage. Dotted line indicates Euclidian distance threshold value used to define the 5 modules of co-expressed genes. Right—list of example genes for each module (modules 1 and 2 merged).

FIGS. 6A-6C illustrate expression of differentially expressed genes in HDM⁺ cells. Standard error dot plots of genes differentially up-regulated in HDM⁺ CD4+ memory effector (T_{H}) and regulatory (T_{reg}) T cells. Each dot represents a sample of 200 cells; shaded indicates the sorted subset. For each panel, the three groups from left to right represent data from HDM⁻, HDM⁺ T_{H}, and HDM⁺ T_{REG}, respectively. Black dots represent mean value expression, error bars represent standard error to the mean expression.

FIGS. 7A-7C illustrate single cell filtering of doublets and low HDM-reactive T cells. (FIG. 7A) Left—t-SNE visualization of all single cell datasets. Centre—t-SNE visualization of cells identified with more than one genotype (dark dots, more than one cell) from the entire single-cell dataset. Right—Demuxlet classification of doublets (droplet with more than 1 genotype identified) as a fraction of all single cell datasets per sequencing library. The left three bars represent data from NA. The fourth to seventh bars counted from the left represent data from AA. The eighth to tenth bards counted from the left represent data from AN. The right three bars under the group of T_{H} represent data from NN. For the group of T_{reg}, the four bars from left to right represent data from NA, AA, AN and NN, respectively. The right most bar represent data from the AA HDM⁻ T group. (FIG. 7B) Upper panels—Volcano plots show differential gene expression analysis between HDM⁻ vs HDM⁺ T_{H} (left), and HDM⁻ vs HDM⁺ T_{reg} (right); dotted lines, which can be extended to a boarder of the boxes, represent threshold of selection: horizontally—Benjamin-Hochberg—adjusted P-value ≤0.05 and vertically, log₂ fold change ≥2. Lower panels—Dot plots represent the distribution of genes ordered based on their mean expression value in function of the percentage of cell expressing the given gene in HDM⁺ T_{H} (left), HDM⁺ T_{reg} (right). Dotted lines, which can be extended to a boarder of the boxes, represent threshold of selection: horizontally, log₂ mean expression ≥0.75 CPM and vertically, fraction of cells expressing the given genes >37.5%. Boxes represent area of selection considered to identify “activation signature genes”. Dark dots in the boxes are selected genes that responded to the 4 criteria of selection listed above. Selected genes are shaded based on the % of cell expressing the given gene (upper panels) and log₂ mean of expression value (lower panels). (FIG. 7C) t-SNE visualization activation signature scores in HDM⁻ with either HDM⁺ T_{H} cells (left), or HDM⁺ T_{reg} cells (right); each dot represents a cell and is shaded based on the score given by AddModuleScore from Seurat.

FIG. 8 illustrates distribution of cells frequency for the 7 HDM⁺ T_{H} clusters for the 24 subjects. Dot plot represents the distribution of log₂ cell count per HDM⁺ T_{H} clusters for each subject. Central bar represents the median and error bars represent inter-quartile distribution of data.

FIG. 9 illustrates co-expression of T_{H}1 or T_{H}17 specific-signature genes. Scatter plots show co-expression of T_{H}1 cluster (top) or T_{H}17 (bottom) cluster cells for canonical signature genes and functionally related genes. On the left of every row, violin plots show distribution of expression for individual genes (shade represent the % of cell expressing the gene). Each scatter plot is split in 4 quadrants (dotted lines at 1 CPM) and percentage of cells present in each quadrant is shown.

FIG. 10 illustrates T_{reg} disease-related differences. Scatter plot shows log₂-fold change in expression of significantly differentially expressed genes between asthma (N=21) versus non-asthma (N=20) (x axis) and allergic (N=22) versus non-allergic (N=19) (y axis) T_{reg} samples.

FIGS. 11A-11F illustrate UMAP clustering analysis of the single-cell transcriptomes of ≈30,000 CD4⁺ T cells isolated from BAL samples of 26 patients with mild and severe asthma. (FIG. 11A) Clusters are marked. (FIG. 11B) Pie chart represents the proportion of cells for each cluster (n=9), T_{H}IFNR cluster is cluster 6 as marked. (FIG. 11C) UMAP show cells enriched in IFN-responsive genes, in circle is the T_{H}IFNR cells. (FIG. 11D) Dot plot illustrates the relative proportion of T_{H}IFNR cells for each subject (median). (FIG. 11E) Violin plot show normalized expression of TNFSF10 in T_{H}IFNR cluster vs other cluster merged. Numbers (%) represent the fraction of cell expressing the gene. (FIG. 11F) UMAP show cells enriched in TRM genes.

FIGS. 12A-12D show that TₕIFNR cells are present in response to other allergens, not just HDM; EXAMPLE 1 examined these cells in HDM-allergic/asthma patients. (FIG. 12A) UMAP clustering analysis of the transcriptomes of ≈2,000 single ASP-reactive CD4+ T cells (CD154+ selected) obtained from 2 mild asthmatic patients. (FIG. 12B) UMAP show cell enriched in IFN-responsive genes, in circle=T_{H}IFNR cells. (FIG. 12C) Pie chart represents the proportion of cells for each cluster (n=9), T_{H}IFNR cluster marked. (FIG. 12D) Violin plot show normalized expression of TNFSF10 in T_{H}IFNR cluster vs other clusters merged. Numbers (%) represent the fraction of cell expressing the gene.

FIGS. 13A-13D detail that TₕIFNR cells are generated in response to viral infections, and this, taken in combination with the fact that TₕIFNR cells are generated against allergens generally as well as viral infection, indicates these cells may dampen airway inflammation. (FIG. 13A) UMAP clustering analysis of the transcriptomes of ≈80,000 single viral-reactive CD4+ T cells (CD154+& CD69+ selected) obtained from 46 individuals. Pie chart represents the proportion of cells for each cluster (n=7), T_{H}IFNR cluster is marked. (FIG. 13B) UMAP show cells highly enriched in IFN-responsive genes, in circle is the T_{H}IFNR cells. (FIG. 13C) Dot plot illustrates the relative proportion of T_{H}IFNR cells for each subject (median). (FIG. 13D) Violin plot show log₂(CPM+1) normalized expression of TNFSF10 in T_{H}IFNR cluster vs all other cluster merged. Numbers (%) represent the fraction of cell expressing the gene.

FIGS. 14A-14D show that TₕIFNR cells are observed as a stable subset in healthy subjects, which was expected to see if they are generated in response to allergens and viruses. (FIG. 14A) UMAP clustering analysis of the single-cell transcriptomes of 700,000 circulating CD4+ T cells stimulated with aCD3/aCD28 (CD69+ selected) obtained from 91 healthy individuals. (FIG. 14B) Pie chart represent the proportion of cells for each cluster (n=16), T_{H}IFNR cluster marked. (FIG. 14C) UMAP plot shows cells enriched in IFN-responsive genes, in circle is the T_{H}IFNR cells as well as T_{REG}. (FIG. 14D) Dot plot illustrates the relative proportion of T_{H}IFNR cells for each subject (median).

FIG. 15 illustrates asthma inflammation and protection models of allergic inflammation with controls, readouts and simplistic representation of expected results; in terms of balance between pathogenic (T_{H}2, 10 pathogenic T cells are exemplified under the T_{H}2 inflammation setting while 5 pathogenic T cells are exemplified under the limited T_{H}2 inf setting) and protective (T_{H}IFNR, marked with a “+”, 1 protective T cell is exemplified under the T_{H}2 inflammation setting while t protective T cells are exemplified under the limited T_{H}2 inf setting) cells.

FIG. 16 provides schematic representation of the different type of experiments outlined in EXAMPLE 2, 1B.

FIG. 17 provides schematic representation of the different type of experiments outlined in EXAMPLE 2, 1C with simplified representation of expected results.

FIGS. 18A-18F provide comparison of both murine models of allergic T_{H}2 airway inflammation implemented, inflammatory (HDM) and protective (HDM/LPS), using ISRE Mx1-GFP reporter mice. (FIG. 18A) Schematic representation of both models. (FIG. 18B) Flow cytometry analysis of Mx1-GFP and CD44 expression of T cells in lung draining lymph nodes. FIGS. 18A-18B detail mouse models of asthma where one is a model that is protective against asthma; the protective model has more TₕIFNR positive cells. (FIG. 18C) Measurement of airway resistance (R) to increasing doses of a standard broncho-constrictor agent, methacholine (MCh) (Flexivent plethysmograph). (FIG. 18D) Flow cytometry measurements of Eosinophils accumulation in upper airways (BAL, broncho-alveolar lavage). (FIG. 18E) Flow cytometry measurements of lung tissue accumulation of memory T_{H}2 cells (ST2+). (FIG. 18F) Flow cytometry measurement of Tₕ2 cytokines, Il13 and Il5, by intra-cellular staining of CD4+ T cells after in vitro stimulation with PMA for 6 hr in presence of brefeldin A (2 hr).

### DETAILED DESCRIPTION

### Definitions

As it would be understood, the section or subsection headings as used herein is for organizational purposes only and are not to be construed as limiting and/or separating the subject matter described.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

As used herein, the singular forms “a”, “an,” and “the” include plural referents unless the context clearly indicates otherwise. For example, the term “a cell” includes a plurality of cells, including mixtures thereof.

As used herein, the term “comprising” is intended to mean that the methods include the recited steps or elements, but do not exclude others. “Consisting essentially of” shall mean rendering the claims open only for the inclusion of steps or elements, which do not materially affect the basic and novel characteristics of the claimed methods. “Consisting of” shall mean excluding any element or step not specified in the claim. Embodiments defined by each of these transition terms are within the scope of this disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

As used herein, comparative terms as used herein, such as high, low, increase, decrease, reduce, elevate or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

As will be understood by one skilled in the art, for any and all purposes, all ranges disclosed herein also encompass any and all possible subranges and combinations of subranges thereof. Furthermore, as will be understood by one skilled in the art, a range includes each individual member.

As used herein, the term “mammal” includes both human and non-human mammals. As used herein, the term “animal” refers to living multi-cellular vertebrate organisms, a category that includes, for example, mammals and birds.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The practice of the present disclosure will employ, unless otherwise indicated, conventional techniques of tissue culture, immunology, molecular biology, microbiology, cell biology and recombinant DNA, which are within the skill of the art. See, e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2nd edition; Guisan ed. (2006) Immobilization of Enzymes and Cells; Perbal (1988) A Practical Guide to Molecular Cloning, 2nd edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4th edition; and Herzenberg et al. eds (1996) Weir's Handbook of Experimental Immunology, 5th edition.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), prophylaxis, progression, amelioration or palliation of the condition (including disease), states and remission (whether partial or total), whether detectable or undetectable. In one aspect, treatment excludes prophylaxis.

Allergy is a condition involving an abnormal immune reaction to an allergen. There are several different types of allergies and allergens, for example, aeroallergens such as dust mite, mold, and pollens; food allergens such as milk, egg, soy, wheat, nut, or fish proteins; allergens from pets or pests; or allergens from drugs. Symptoms of allergy include, but are not limited to, sneezing, runny or stuffy nose, red, itchy or teary eyes, wheezing, coughing, tightness in the chest and shortness of breath, or itching. In some instances, an allergic response comprises an inflammation of the skin, airway mucosa, or a combination thereof. In some instances, an allergic response comprises atopic dermatitis, allergic rhinitis, allergic asthma, allergic conjunctivitis, or a combination thereof.

House dust mites (HDM; Dermatophagoides spp.) are members of the mite family Pyroglyphidae with D. pteronyssinus (European HDM), D. farina (American HDM), D. microcera, D. siboney, Blomia tropicalis, Euroglyphus maynei (Mayne's HDM), Psoroptes ovis, Sarcoptes scabie, and Lepidoglyphus destructor as the main species. HDM allergens comprise proteins secreted in the fecal pellets of HDMs and include for example, but are not limited to, cysteine protease or papain-like proteins such as Der p 1, Der f 1, Der m 1, Der s 1, Eur m 1, Blo t 1, Pso o 1, and Sar s 1; trypsin-like serine proteases such as Der p 3, Der f 3, Der s 3, Eur m 3, Blot 3, Sar s 3, Gly d 3, and Lep d 3; chymotrypsin-like serine proteases such as Der p 6, Der f 6, and Blo t 6; and collagenolytic-like serine proteases such as Der p 9, Der f 9, and Blo t 9. (Note the allergen nomenclature is as follows: Der p, Dermatophagoides pteronyssinus; Der f, D. farinae; Der m, D. microcera; Der s, D. siboney; Eur m, Euroglyphus maynei; Blo t, Blomia tropicalis; Pso o, Psoroptes ovis; Sar s, Sarcoptes scabie; Lep d, Lepidoglyphus destructor.)

As used herein, a HDM peptide comprises a HDM allergenic peptide, e.g., from a protein secreted in the fecal pellets of the HDMs. In some instances, the HDM peptide comprises a fragment of a cysteine protease or papain-like protein such as Der p 1, Der f 1, Der m 1, Der s 1, Eur m 1, Blot 1, Pso o 1, or Sar s 1; a trypsin-like serine protease such as Der p 3, Der f 3, Der s 3, Eur m 3, Blot 3, Sar s 3, Gly d 3, or Lep d 3; a chymotrypsin-like serine protease such as Der p 6, Der f 6, or Blo t 6; or a collagenolytic-like serine protease such as Der p 9, Der f 9, or Blo t 9.

As used herein, a HDM-induced allergy refers to an allergic response in a subject in the presence of HDM allergens or has come into contact with one or more HDM allergens. In some instances, the HDM-induced allergy comprises an inflammation of the skin, airway mucosa, or a combination thereof. In some instances, the HDM-induced allergy comprises symptoms of sneezing, runny or stuffy nose, red, itchy or teary eyes, wheezing, coughing, tightness in the chest and shortness of breath, or itching. In some instances, the HDM-induced allergy comprises atopic dermatitis, allergic rhinitis, allergic asthma, allergic conjunctivitis, or a combination thereof.

Atopic dermatitis or AD, is the most common type of eczema. AD is characterized with red, itchy rash on the cheeks, arms, and/or legs. AD is chronic, and symptoms include dry, scaly skin; redness; itching; cracks behind the ear; rash on the cheeks, arms and/or legs; and/or open, crusted or “weepy” sores.

Allergic rhinitis (also known as hay fever) is the most common type of chronic rhinitis. Allergic rhinitis is characterized with inflammatory of the respiratory tract, with accumulations of phagocytes, eosinophils, mast cells, and lymphocytes, and elevated levels of serum IgE and/or allergen-specific IgE. Symptoms of allergic rhinitis include sneezing, congestion, coughing, sinus pressure, itchy watery eyes, and itchy nose, mouth, and throat.

Allergic asthma (or allergy-induced asthma) is the most common type of asthma. Symptoms of allergic asthma include chest tightness, coughing, difficulty in breathing, shortness of breath, and wheezing. In some instances, patients suffering from allergic asthma also experience atopic dermatitis, allergic conjunctivitis, and/or allergic rhinitis.

Allergic conjunctivitis is an inflammation of the conjunctiva caused by an allergen. Symptoms of allergic conjunctivitis may be acute, seasonal, or perennial, and can include itching, redness, tearing, and/or swelling of the eyelid. In some instances, symptoms also include sensitivity to light and/or burning. Patients with allergic conjunctivitis may also suffer from eczema, allergic rhinitis, and/or allergic asthma.

T helper cells (also referred to herein Th cells, Tₕ cells, T_{H} cells or T_{H} cells), also known as CD4+ T cells, are mature T cells that are involved in modulating the immune system, in particular, the adaptive immune system. There are two subtypes of Th cells, T helper 1 (T_{H}1) cells and T helper 2 (T_{H}2) cells. T_{H}2 cells represent a distinct population of T cells that regulate type 2 (Th2) responses through the secretion of cytokines such as IL-4, IL-5, IL-10, and IL-13. T_{H}2 cells have been associated with inducing allergic/inflammatory responses observed in allergic rhinitis, atopic dermatitis, and allergic asthma, and allergic conjunctivitis. Cytokines such as IL-25 and IL-33, thymic stromal lymphopoietin (TSLP), and IL-4 producing immune cells such as innate lymphoid cells and basophils can potentiate T_{H}2 cell responses. CD154, also known as CD40 ligand or CD40L, is a surface marker expressed on mature CD4+ T cells such as T_{H}2 cells. As used herein, a HDM-reactive T_{H} cell (or HDM-reactive T_{H}2 cell) is CD154⁺.

Regulatory T (also referred to herein as T_{reg}, T_{reg}, T_{REG} or T_{REG}) cells are CD4+ T cells involved in maintaining peripheral tolerance, preventing autoimmunity, and limiting chronic inflammatory diseases. T_{reg} cells have been shown to suppress inflammation by upregulating immunosuppressive molecules and tissue homing receptors and repressing genes. There are five subtypes of T_{reg} cells based on the expression of the transcription factor FOXP3. FOXP3⁺ T_{reg} cells include thymus-derived T_{reg} (tT_{reg}) cells and peripheral regulatory T cells (pT_{reg} cells). The FOXP3⁻ T_{reg} cells include Tr1, Th3, and CD8⁺ T_{reg} cells. CD137 (also known as 4-1BB or tumor necrosis factor receptor 9, TNFR9) is a member of the TNFR family expressed on activated T cells, NK cells, dendritic cells, eosinophils, mast cells, and endothelial cells. In some instances, T_{reg} cells express CD137 (or is CD137⁺) but does not express CD154 (or is CD154⁻). As used herein, a HDM-reactive T_{reg} cell is CD154⁻ and CD137⁺.

As used herein, a HDM non-reactive T cell is CD154⁻ and CD137⁻.

CD154, which is also referred to herein as CD40L, is a protein that is primarily expressed on activated T cells and is a member of the TNF superfamily of molecules. In total CD40L has three binding partners: CD40, α5β1 integrin and αIIbβ3. In some embodiments, the CD154 is a human CD154. Non-limiting exemplary sequences of this protein or the underlying gene can be found under Gene Cards ID: GC0XP136649, HGNC: 11935, NCBI Entrez Gene: 959, Ensembl: ENSG00000102245, OMIM®: 300386, and UniProtKB/Swiss-Prot: P29965, each of which is incorporated by reference herein in its entirety.

CD137 is also referred to herein as TNF Receptor Superfamily Member 9 or 4-1BB. CD137 is expressed by activated T cells of both the CD4+ and CD8+ lineages. Although it is thought to function mainly in co-stimulating those cell types to support their activation by antigen presenting cells expressing its ligand (CD137L), CD137 is also expressed on dendritic cells, B cells, NK cells, neutrophils and macrophages. In some embodiments, the CD137 is a human CD137. Non-limiting exemplary sequences of this protein or the underlying gene can be found under Gene Cards ID: GC01M007915, HGNC: 11924, NCBI Entrez Gene: 3604, Ensembl: ENSG00000049249, OMIM®: 602250, and UniProtKB/Swiss-Prot: Q07011, each of which is incorporated by reference herein in its entirety.

As used herein, “enrich” refers to an increase the concentration or amount of one substance, such as a cell expressing a marker, optionally relative to the concentration or amount of another substance; or one composition containing a higher concentration or amount of a substance, compared to a second composition's concentration or amount of that substance. The difference in the amount (weight/mass/number) can be at least about 1%, at least about 5%, at least about 10%, at least about 25%, or at least about 50%. Likewise, the difference in concentration can be at least about 1%, at least about 5%, at least about 10%, at least about 25%, or at least about 50%. In reference to “higher concentration” and “lower concentration,” the difference in concentration can be at least about 1%, at least about 5%, at least about 10%, at least about 25%, or at least about 50%.

The term “expression level” refers to protein, RNA, or mRNA level of a particular gene of interest. Any methods known in the art can be utilized to determine the expression level of a particular gene of interest. Examples include, but are not limited to, reverse transcription and amplification assays (such as PCR, ligation RT-PCR or quantitative RT-PCT), hybridization assays, Northern blotting, dot blotting, in situ hybridization, gel electrophoresis, capillary electrophoresis, column chromatography, Western blotting, immunohistochemistry, immunostaining, or mass spectrometry. Assays can be performed directly on biological samples or on protein/nucleic acids isolated from the samples. It is routine practice in the relevant art to carry out these assays. For example, the detecting step in any method described herein includes contacting the nucleic acid sample from the biological sample obtained from the subject with one or more primers that specifically hybridize to the gene of interest presented herein. Alternatively, the detecting step of any method described herein includes contacting the protein sample from the biological sample obtained from the subject with one or more antibodies that bind to the gene product of the interest presented herein. In some embodiment, the level is an absolute amount or concentration of the protein, RNA, or mRNA level of a particular gene of interest in a cell. In some embodiment, the level is normalized to a control, such as a housekeeping gene (i.e., a constitutive gene that are required for the maintenance of basic cellular function, and is expressed in all cells of an organism under normal and pathophysiological conditions, such as TUBB) in the cell.

The term “gene” refers to a nucleic acid (for example, DNA or RNA) sequence that comprises coding sequences necessary for the production of a gene product, such as a RNA, or a polypeptide or its precursor. In some embodiments, a gene product may be presented by the gene name as used herein.

As used herein, the term “gene profile” refers to information of expression level of a gene. The gene profile can comprise data for one or more genes and can be measured at a single time point or over a period of time. In some embodiments, the gene profile comprises, or consists essentially of, or yet further consists of the expression level of the gene(s). In some embodiments, the gene profile comprises, or consists essentially of, or yet further consists of elevated or significantly similar to or decreased expression level of the gene(s).

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

As used herein, an antigen-specific T cell is a Th cell or a T_{reg} cell, which has been activated in the presence of an allergen. In some instances, the allergen is an aeroallergen such as dust mite, mold, and a pollen; a food allergen such as milk, egg, soy, wheat, nut, or fish protein; an allergen from a pet or a pest; or an allergen from a drug. In some instances, the allergen is a HDM allergen. In some cases, the HDM allergen comprises a protein secreted in the fecal pellets of HDMs and includes for example, but is not limited to, a cysteine protease or papain-like protein such as Der p 1, Der f 1, Der m 1, Der s 1, Eur m 1, Blot 1, Pso o 1, and Sar s 1; a trypsin-like serine protease such as Der p 3, Der f 3, Der s 3, Eur m 3, Blo t 3, Sar s 3, Gly d 3, and Lep d 3; a chymotrypsin-like serine protease such as Der p 6, Der f 6, and Blo t 6; or a collagenolytic-like serine protease such as Der p 9, Der f 9, and Blo t 9.

TNF superfamily member 10 (or TNFSF10) encodes the protein TRAIL. TRAIL is a type II transmembrane protein that can induce apoptosis upon interaction with its receptors including death receptor 4 (DR4), death receptor 5 (DR5) (or KILLER), decoy receptor 1 (DcR1) (or TRID, TRAIL-R3), decoy receptor 2 (DcR2) (or TRAIL-R4), and osteoprotegerin (OPG). In some instances and under a HDM-induced allergy setting, TRAIL dampens or decreases allergic response induced by HDM in a subject in need thereof. Synonyms of TNFSF10 include Apo-2 ligand, Apo-2L, APO2L, tumor necrosis factor apoptosis-inducing ligand splice variant delta, chemokine tumor necrosis factor ligand superfamily member 10, CD253 antigen, TNLG6A, and TL2. In some embodiments, the TRAIL is a human TRAIL. Non-limiting exemplary sequences of this protein or the underlying gene can be found under Gene Cards ID: GC03M172505, HGNC: 11925, NCBI Entrez Gene: 8743, Ensembl: ENSG00000121858, OMIM®: 603598, and UniProtKB/Swiss-Prot: P50591, each of which is incorporated by reference herein in its entirety. As used herein, a recombinant TRAIL protein comprises both full-length TRAIL protein and fragments thereof. In some instances, fragments of TRAIL comprises a functional fragment, a fragment that binds to one or more of its receptors and/or induces apoptosis upon receptor binding. In some instances, a TRAIL fragment comprises the extracellular portion, e.g., residues 39-281 of SEQ ID NO: 1, or an equivalent thereof. In some instances, TRAIL is a wild-type protein, or a variant thereof. In some cases, TRAIL comprises the amino acid sequence as set forth in SEQ ID NO: 1.

(SEQ ID NO: 1)
MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGTRGRSNTLSSPNSKNEKALGRK
INSWESSRSGHSFLSNLHLRNGELVIHEKGFYYIYSQTYFRFQEEIKENT
KNDKQMVQYIYKYTSYPDPILLMKSARNSCWSKDAEYGLYSIYQGGIFEL
KENDRIFVSVTNEHLIDMDHEASFFGAFLVG
(UniProtKB: P50591).

In some instances, TRAIL is a human TRAIL (e.g., from Miltenyi Biotec; Cat. No. 130-094-025).

As used herein, an equivalent thereof in reference to SEQ ID NO: 1 comprises a polypeptide comprising at least 80%, 85%, 90%, 95%, 96%, 97%, 98%, or 99% sequence identity to SEQ ID NO: 1, while still retaining a functional activity. In some instances, the functional activity refers to binding with one or more of the receptors, e.g., including but not limited to death receptor 4 (DR4), death receptor 5 (DR5) (or KILLER), decoy receptor 1 (DcR1) (or TRID, TRAIL-R3), decoy receptor 2 (DcR2) (or TRAIL-R4), and osteoprotegerin (OPG). In other instances, the functional activity refers to inducing apoptosis upon receptor binding.

C—X—C motif chemokine ligand 10 (or CXCL10) is encodes by the CXCL10 gene. CXCL10 is a cytokine that belongs to the CXC chemokine family. CXCL10 binds to receptor CXCR3, which subsequently induces stimulation of monocytes, nature killer and T-cell migration, and modulation of adhesion molecule expression. Synonyms of CXCL10 include small-inducible cytokine B10, 10 KDa interferon gamma-induced protein, SCYB10, INP10, IP-10, gamma IP10, GIP-10, IFI10, Crg-2, or Mob-1. In some embodiments, the CXCL10 is a human CXCL10. Non-limiting exemplary sequences of this protein or the underlying gene can be found under Gene Cards ID: GC04M076021, HGNC: 10637, NCBI Entrez Gene: 3627, Ensembl: ENSG00000169245, OMIM®: 147310, and UniProtKB/Swiss-Prot: P02778, each of which is incorporated by reference herein in its entirety.

Interferon Alpha Inducible Protein (IFI6) is encodes by the IFI6 gene. The encoded protein may play a critical role in the regulation of apoptosis. In some embodiments, the IFI6 is a human IFI6. Non-limiting exemplary sequences of this protein or the underlying gene can be found under Gene Cards ID: GC01M027666, HGNC: 4054, NCBI Entrez Gene: 2537, Ensembl: ENSG00000126709, OMIM®: 147572, and UniProtKB/Swiss-Prot: P09912, each of which is incorporated by reference herein in its entirety.

MX Dynamin Like GTPase 1 (MX1) is encodes by the MX1 gene. The MX1 is responsible for a specific antiviral state against influenza virus infection. Furthermore, the human orthologue MxA is a major determinant for influenza viruses of animal origin. In some embodiments, the MX1 is a human MX1. Non-limiting exemplary sequences of this protein or the underlying gene can be found under Gene Cards ID: GC21P041420, HGNC: 7532, NCBI Entrez Gene: 4599, Ensembl: ENSG00000157601, OMIM®: 147150 and UniProtKB/Swiss-Prot: P20591, each of which is incorporated by reference herein in its entirety.

ISG15 Ubiquitin Like Modifier (ISG15) is encodes by the ISG15 gene. ISG15 is induced by type I interferon (IFN) and serves many functions, acting both as an extracellular cytokine and an intracellular protein modifier. The precise functions are diverse and vary among species but include potentiation of Interferon gamma (IFN-II) production in lymphocytes, ubiquitin-like conjugation to newly-synthesized proteins and negative regulation of the IFN-I response. In some embodiments, the ISG15 is a human ISG15. Non-limiting exemplary sequences of this protein or the underlying gene can be found under Gene Cards ID: GC01P001001, HGNC: 4053, NCBI Entrez Gene: 9636, Ensembl: ENSG00000187608, OMIM®: 147571, and UniProtKB/Swiss-Prot: P05161, each of which is incorporated by reference herein in its entirety.

Interferon Stimulated Exonuclease Gene 20 (ISG20) is encodes by the ISG20 gene. It is an interferon-induced antiviral exoribonuclease that acts on single-stranded RNA and also has minor activity towards single-stranded DNA. It exhibits antiviral activity against RNA viruses including hepatitis C virus (HCV), hepatitis A virus (HAV) and yellow fever virus (YFV) in an exonuclease-dependent manner. ISG20 may also play additional roles in the maturation of snRNAs and rRNAs, and in ribosome biogenesis. In some embodiments, the ISG20 is a human ISG20. Non-limiting exemplary sequences of this protein or the underlying gene can be found under Gene Cards ID: GC15P088635, HGNC: 6130, NCBI Entrez Gene: 3669, Ensembl: ENSG00000172183, OMIM®: 604533, and UniProtKB/Swiss-Prot: Q96AZ6, each of which is incorporated by reference herein in its entirety.

2′-5′-Oligoadenylate Synthetase 1 (OAS1) is encodes by the OAS1 gene. This gene encodes a member of the 2-5A synthetase family, essential proteins involved in the innate immune response to viral infection. The encoded protein is induced by interferons and uses adenosine triphosphate in 2′-specific nucleotidyl transfer reactions to synthesize 2′,5′-oligoadenylates (2-5As). These molecules activate latent RNase L, which results in both viral and endogenous RNA degradation and the inhibition of viral replication. The three known members of this gene family are located in a cluster on chromosome 12. Mutations in this gene have been associated with host susceptibility to viral infection. Alternatively spliced transcript variants encoding different isoforms have been described. In some embodiments, the OAS1 is a human OAS1. Non-limiting exemplary sequences of this protein or the underlying gene can be found under Gene Cards ID: GC12P112911, HGNC: 8086, NCBI Entrez Gene: 4938, Ensembl: ENSG00000089127, OMIM®: 164350, and UniProtKB/Swiss-Prot: P00973, each of which is incorporated by reference herein in its entirety.

2′-5′-Oligoadenylate Synthetase 3 (OAS3) is encodes by the OAS3 gene. This gene encodes an enzyme included in the 2′,5′ oligoadenylate synthase family. This enzyme is induced by interferons and catalyzes the 2′,5′ oligomers of ATP. These oligomers activate latent RNase L, leading to degradation of both viral and endogenous RNA. This enzyme family plays a significant role in the inhibition of cellular protein synthesis in response to viral infection. In some embodiments, the OAS3 is a human OAS3. Non-limiting exemplary sequences of this protein or the underlying gene can be found under Gene Cards ID: GC12P112938, HGNC: 8088, NCBI Entrez Gene: 4940, Ensembl: ENSG00000111331, OMIM®: 603351, and UniProtKB/Swiss-Prot: Q9Y6K5, each of which is incorporated by reference herein in its entirety.

Interferon Induced Protein With Tetratricopeptide Repeats 1 (IFIT1) is encodes by the IFIT1 gene. The encoded protein may inhibit viral replication and translational initiation. Alternatively spliced transcript variants encoding multiple isoforms have been observed. In some embodiments, the IFIT1 is a human IFIT1. Non-limiting exemplary sequences of this protein or the underlying gene can be found under Gene Cards ID: GC10P089396, HGNC: 5407, NCBI Entrez Gene: 3434, Ensembl: ENSG00000185745, OMIM®: 147690, and UniProtKB/Swiss-Prot: P09914, each of which is incorporated by reference herein in its entirety.

Interferon Induced Protein With Tetratricopeptide Repeats 3 (IFIT3) is encodes by the IFIT3 gene. It is an IFN-induced antiviral protein which acts as an inhibitor of cellular as well as viral processes, cell migration, proliferation, signaling, and viral replication. In some embodiments, the IFIT3 is a human IFIT3. Non-limiting exemplary sequences of this protein or the underlying gene can be found under Gene Cards ID: GC10P089327, HGNC: 5411, NCBI Entrez Gene: 3437, Ensembl: ENSG00000119917, OMIM®: 604650, and UniProtKB/Swiss-Prot: 014879, each of which is incorporated by reference herein in its entirety.

Interferon Induced Transmembrane Protein 1 (IFITM1) is encodes by the IFITM1 gene. IFITM1 is a member of the IFITM family (Interferon-induced transmembrane protein). The human IFITM genes locate on chromosome 11 and have four members: IFITM1, IFITM2, IFITM3 and IFITM5. In some embodiments, the IFITM1 is a human IFITM1. Non-limiting exemplary sequences of this protein or the underlying gene can be found under Gene Cards ID: GC11P000313, HGNC: 5412, NCBI Entrez Gene: 8519, Ensembl: ENSG00000185885, OMIM®: 604456, and UniProtKB/Swiss-Prot: P13164, each of which is incorporated by reference herein in its entirety.

Interferon Induced Protein 44 Like (IFI44L) is encodes by the IFI44L gene. It exhibits a low antiviral activity against hepatitis C virus. In some embodiments, the IFI44L is a human IFI44L. Non-limiting exemplary sequences of this protein or the underlying gene can be found under Gene Cards ID: GC01P078619, HGNC: 17817, NCBI Entrez Gene: 10964, Ensembl: ENSG00000137959, OMIM®: 613975, and UniProtKB/Swiss-Prot: Q53G44, each of which is incorporated by reference herein in its entirety.

Sterile Alpha Motif Domain Containing 9 Like (SAMDL9), which is also referred to herein as SAMD9L is encodes by the SAMDL9 gene. It is involved in endosome fusion and mediates down-regulation of growth factor signaling via internalization of growth factor receptors. In some embodiments, the SAMDL9 is a human SAMDL9. Non-limiting exemplary sequences of this protein or the underlying gene can be found under Gene Cards ID: GC07M093130, HGNC: 1349, NCBI Entrez Gene: 219285, Ensembl: ENSG00000177409, OMIM®: 611170, and UniProtKB/Swiss-Prot: Q8IVG5, each of which is incorporated by reference herein in its entirety.

X—C Motif Chemokine Ligand 2 (XCL2) is encodes by the XCL2 gene. It is a small cytokine belonging to the XC chemokine family that is highly related to another chemokine called XCL1. It is predominantly expressed in activated T cells, but can also be found at low levels in unstimulated cells. XCL2 induces chemotaxis of cells expressing the chemokine receptor XCR1. In some embodiments, the XCL2 is a human XCL2. Non-limiting exemplary sequences of this protein or the underlying gene can be found under Gene Cards ID: GC01M168510, HGNC: 10646, NCBI Entrez Gene: 6846, Ensembl: ENSG00000143185, OMIM®: 604828, and UniProtKB/Swiss-Prot: Q9UBD3, each of which is incorporated by reference herein in its entirety.

In some embodiments, Semaphorin 7A (John Milton Hagen Blood Group) (SEMA7A), which is also referred to herein as SEMATA, is encodes by the SEMA7A gene. SEMA7A is a membrane-bound semaphorin that associates with cell surfaces via a glycosylphosphatidylinositol (GPI) linkage. SEMA7A is also known as the John-Milton-Hagen (JMH) blood group antigen, an 80-kD glycoprotein expressed on activated lymphocytes and erythrocytes. SEMA7A is expressed in various adult tissues such as adipose, colon, esophagus, heart, brain, spleen, testis, lung, ovary, and uterus. In some embodiments, the SEMA7A is a human SEMA7A. Non-limiting exemplary sequences of this protein or the underlying gene can be found under Gene Cards ID: GC15M074409, HGNC: 10741, NCBI Entrez Gene: 8482, Ensembl: ENSG00000138623, OMIM®: 607961, and UniProtKB/Swiss-Prot: 075326, each of which is incorporated by reference herein in its entirety.

C—X—C Motif Chemokine Receptor 3 (CXCR3) is encodes by the CXCR3 gene. It is a Gai protein-coupled receptor in the CXC chemokine receptor family. There are three isoforms of CXCR3 in humans: CXCR3-A, CXCR3-B and chemokine receptor 3-alternative (CXCR3-alt). CXCR3-A binds to the CXC chemokines CXCL9 (MIG), CXCL10 (IP-10), and CXCL11 (I-TAC) whereas CXCR3-B can also bind to CXCL4 in addition to CXCL9, CXCL10, and CXCL11. In some embodiments, the CXCR3 is a human CXCR3. Non-limiting exemplary sequences of this protein or the underlying gene can be found under Gene Cards ID: GCOXM071615, HGNC: 4540, NCBI Entrez Gene: 2833, Ensembl: ENSG00000186810, OMIM®: 300574, and UniProtKB/Swiss-Prot: P49682, each of which is incorporated by reference herein in its entirety.

Fas Ligand (FASLG) is encodes by the FASLG gene. It is a type-II transmembrane protein that belongs to the tumor necrosis factor (TNF) family. Its binding with its receptor induces apoptosis. Fas ligand/receptor interactions play an important role in the regulation of the immune system and the progression of cancer. In some embodiments, the FASLG is a human FASLG. Non-limiting exemplary sequences of this protein or the underlying gene can be found under Gene Cards ID: GC01P172628, HGNC: 11936, NCBI Entrez Gene: 356, Ensembl: ENSG00000117560, OMIM®: 134638, and UniProtKB/Swiss-Prot: P48023, each of which is incorporated by reference herein in its entirety.

Interferon Gamma (IFNG) is encodes by the IFNG gene. It is a dimerized soluble cytokine that is the only member of the type II class of interferons. In addition to having antiviral activity, it has important immunoregulatory functions, and is a potent activator of macrophages. In some embodiments, the IFNG is a human IFNG. Non-limiting exemplary sequences of this protein or the underlying gene can be found under Gene Cards ID: GC12M068154, HGNC: 5438, NCBI Entrez Gene: 3458, Ensembl: ENSG00000111537, OMIM®: 147570, and UniProtKB/Swiss-Prot: P01579, each of which is incorporated by reference herein in its entirety.

Perforin 1 (PRF1) is encodes by the PRF1 gene. It is a pore-forming protein that plays a key role in secretory granule-dependent cell death, and in defense against virus-infected or neoplastic cells. In some embodiments, the PRF1 is a human PRF1. Non-limiting exemplary sequences of this protein or the underlying gene can be found under Gene Cards ID: GC10M070597, HGNC: 9360, NCBI Entrez Gene: 5551, Ensembl: ENSG00000180644, OMIM®: 170280, and UniProtKB/Swiss-Prot: P14222, each of which is incorporated by reference herein in its entirety.

Killer Cell Lectin Like Receptor G1 (KLRG1) is encodes by the KLRG1 gene. In some embodiments, the KLRG1 is a human KLRG1. Non-limiting exemplary sequences of this protein or the underlying gene can be found under Gene Cards ID: GC12P008950, HGNC: 6380, NCBI Entrez Gene: 10219, Ensembl: ENSG00000139187, OMIM®: 604874, and UniProtKB/Swiss-Prot: Q96E93, each of which is incorporated by reference herein in its entirety.

X—C Motif Chemokine Ligand 1 (XCL1) is encodes by the XCL1 gene. It is a small cytokine belonging to the C chemokine family that is also known as lymphotactin. Lymphotactins can go through a reversible conformational change which changes its binding shifts In some embodiments, the XCL1 is a human XCL1. Non-limiting exemplary sequences of this protein or the underlying gene can be found under Gene Cards ID: GC01P168576, HGNC: 10645, NCBI Entrez Gene: 6375, Ensembl: ENSG00000143184, OMIM®: 600250, and UniProtKB/Swiss-Prot: P47992, each of which is incorporated by reference herein in its entirety.

CD226 is encodes by the CD226 gene. This gene encodes a glycoprotein expressed on the surface of NK cells, platelets, monocytes and a subset of T cells. It is a member of the Ig-superfamily containing 2 Ig-like domains of the V-set. The protein mediates cellular adhesion of platelets and megakaryocytic cells to vascular endothelial cells. The protein also plays a role in megakaryocytic cell maturation. Alternative splicing results in multiple transcript variants. In some embodiments, the CD226 is a human CD226. Non-limiting exemplary sequences of this protein or the underlying gene can be found under Gene Cards ID: GC18M069831, HGNC: 16961, NCBI Entrez Gene: 10666, Ensembl: ENSG00000150637, OMIM®: 605397, and UniProtKB/Swiss-Prot: Q15762, each of which is incorporated by reference herein in its entirety.

Nuclear Factor Of Activated T Cells 1 (NFATC1) is encodes by the NFATC1 gene. The product of this gene is a component of the nuclear factor of activated T cells DNA-binding transcription complex. This complex consists of at least two components: a preexisting cytosolic component that trans-locates to the nucleus upon T cell receptor (TCR) stimulation, and an inducible nuclear component. In some embodiments, the NFATC1 is a human NFATC1. Non-limiting exemplary sequences of this protein or the underlying gene can be found under Gene Cards ID: GC18P079395, HGNC: 7775, NCBI Entrez Gene: 4772, Ensembl: ENSG00000131196, OMIM®: 600489, and UniProtKB/Swiss-Prot: 095644, each of which is incorporated by reference herein in its entirety.

Interleukins are a group of cytokines expressed by leukocytes. IL-5 (encoded by the IL-5 gene) and IL-9 (encoded by the IL-9 gene), are two interleukins that have been observed to be elevated in asthmatic/allergic patients.

Interleukin 1 receptor like 1 (IL1RL1), encoded by the IL1RL1 gene, a member of the interleukin 1 receptor family. IL1RL1, along with the IL-1 receptor accessory protein IL1RAcP, interacts with IL-33 and the receptor complex induces through activation of the signaling proteins release of allergic and eosinophilic mediators such as IL-5 and IL-13, resulting in esosinophilic inflammation and attenuation of the IL-33 signal. Synonyms of IL1RL1 include DER4, ST2, FIT-1, IL33R, and ST2L.

Zinc finger E-box binding homeobox 2 (ZEB2), encoded by the ZEB2 gene, is a member of the Zfh1 family of 2-handed zinc finger/homeodomain proteins. The ZEB2 protein is a transcription factor that is involved in transforming growth factor β (TGFβ) signaling pathways involved in early fetal development. Synonyms of ZEB2 include smad-interacting protein 1, SMADIP1, SIP1, or ZFX1B.

Granzyme B (or GrB) is encoded by the GZMB gene. GrB is a serine protease found in the granules of natural killer cells (NK) cells, cytotoxic T cells, and memory T cells to mediate cellular apoptosis. Synonyms of GZMB include cytotoxic serine protease B, CTSGL1, CTLA1, CSPB, or CGL1.

MAF BZIP transcription factor (MAF), encoded by the MAF gene, is a DNA-binding, leucine zipper-containing transcription factor. The MAF protein is involved in increased T-cell susceptibility to apoptosis. Synonyms of MAF include proto-oncogene C-Maf, avian musculoaponeurotic fibrosarcoma (MAF) protooncogene, AYGRP, C-MAF, and CCA4.

Mitogen-activated protein kinase kinase kinase 8 (MAP3K8), encoded by the MAP3K8 gene, is a member of the serine/threonine protein kinase family. Synonyms of MAP3K8 include tumor progression locus 2, proto-oncogene C-Cot, ESTF, COT, or EST.

Mitochondrially encoded NADH:Ubiquinone oxidoreductase core subunit 5 (or MT-ND5) encodes the NADH-ubiquinone oxidoreductase chain 5 (ND5) protein. Synonyms of MT-ND5 include NADH dehydrogenase subunit 5 and complex I ND5 subunit.

Dual specificity phosphatase 6 (DUSP6), encoded by the DUSP6 gene, is an enzyme belonging to the dual specificity protein phosphatase subfamily. Synonyms of DUSP6 include dual specificity protein phosphatase PYST1, MAP kinase phosphatase 3, PYST1, MKP-3, and HH19.

SEC61 translocon subunit gamma (encoded by SEC61G) and SEC61 translocon subunit beta (encoded by SEC61B) are members of the protein translocation apparatus of the endoplasmic reticulum (ER) membrane.

DnaJ heat shock protein family (Hsp40) member C3 is encoded by the DNAJC3 gene. Synonyms of DNAJC3 include interferon-induced, double-stranded RNA-activated protein kinase inhibitor, endoplasmic reticulum DNA J domain-containing protein 6, protein kinase inhibitor of 58 KDa, P58IPK, PRKRI, ERdj6, ACPHD, or HP58.

Abelson helper integration site 1 is encoded by the AHI1 gene. Synonyms of AHI1 include DJ71N10.1, JBTS3, and ORF1.

Cyclin dependent kinase 2 associated protein 2 is encoded by the CDK2AP2 gene. Synonyms of CDK2AP2 include DOC-1-related protein, DOC-1R, and P14.

FKBP prolyl isomerase 11 (encoded by FKBP11) and FKBP prolyl isomerase 1A (encoded by FKBP1A) are members of the immunophilin protein family, which are involved in immunoregulation and cellular processes involving protein folding and trafficking.

KDEL endoplasmic reticulum protein retention receptor 2, encoded by the KDELR2 gene, is a member of the KDEL receptor gene family. Synonyms of KDELR2 include ERD2.2, ELP-1, and ERD-2-like protein.

Cluster of Differentiation 109, encoded by the CD109 gene, is a GPI-liked cell surface antigen expressed by CD34+ acute myeloid leukemia cell lines, T-cell lines, activated T lymphoblasts, endothelial cells, and activated platelets. Synonyms of CD109 include C3 and PZP-like alpha-2-macroglobulin domain-containing protein 7, 150 KDa TGF-Beta-1-binding protein, platelet-specific Gov antigen, CPAMD7, P180, and R150.

As used herein, the term “biological sample” comprise a fluid sample. Exemplary fluid samples include blood sample, serum sample, plasma sample, saliva sample, urine sample, and tear sample. In some instances, the biological sample comprises a blood sample, e.g., a whole blood sample or a peripheral blood sample or a peripheral blood mononuclear cell (PBMC) sample. In some cases, the biological sample comprises a PBMC sample. Other non-exemplary samples include, but are not limited to, ocular fluids (aqueous and vitreous humor), aqueous humor, vitreous humor, ascites, cerebrospinal fluid (CSF), sputum, bone marrow, synovial fluid, amniotic fluid, cerumen, breast milk, broncheoalveolar lavage fluid, semen, prostatic fluid, cowper's fluid or pre-ejaculatory fluid, female ejaculate, sweat, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR)” the “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains.

As used herein, the term “antigen binding domain” refers to any protein or polypeptide domain that can specifically bind to an antigen target.

As used herein, the term “CD8 a hinge domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 a hinge domain sequence as shown herein. The example sequences of CD8 a hinge domain for human, mouse, and other species are provided in Pinto, R. D. et al. (2006) Vet. Immunol. Immunopathol. 1 10: 169-177. The sequences associated with the CD8 a hinge domain are provided in Pinto, R. D. et al. (2006) Vet. Immunol. Immunopathol. 1 10: 169-177.

As used herein, the term “CD8 a transmembrane domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 a transmembrane domain sequence as shown herein. The fragment sequences associated with the amino acid positions 183 to 203 of the human T-cell surface glycoprotein CD8 alpha chain (GenBank Accession No: NP OO 1759.3), or the amino acid positions 197 to 217 of the mouse T-cell surface glycoprotein CD8 alpha chain (GenBank Accession No: NP 001074579.1), and the amino acid positions 190 to 210 of the rat T-cell surface glycoprotein CD8 alpha chain (GenBank Accession No: NP_1 13726.1) provide additional example sequences of the CD8 a transmembrane domain.

As used herein, the term “4-IBB costimulatory signaling region” refers to a specific protein fragment associated with this name and any other molecules that have analogous biological function that share at least 70%, or alternatively at least 80%>amino acid sequence identity, preferably 90% sequence identity, more preferably at least 95% sequence identity with the 4-IBB costimulatory signaling region sequence as shown herein. Non-limiting example sequences of the 4-IBB costimulatory signaling region are provided in U.S. Publication 20130266551 A1.

As used herein, the term “ICOS costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the ICOS costimulatory signaling region sequence as shown herein. Non-limiting example sequences of the ICOS costimulatory signaling region are provided in U.S. Patent Application Publication No. 2015/0017141 A1.

As used herein, the term “OX40 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the OX40 costimulatory signaling region sequence as shown herein. Non-limiting example sequences of the OX40 costimulatory signaling region are disclosed in U.S. Patent Application Publication No. 2012/20148552A1.

As used herein, the term “CD28 transmembrane domain” refers to a specific protein fragment associated with this name and any other molecules that have analogous biological function that share at least 70%, or alternatively at least 80%>amino acid sequence identity, at least 90% sequence identity, or alternatively at least 95% sequence identity with the CD28 transmembrane domain sequence as shown herein. The fragment sequences associated with the GenBank Accession Nos: XM_006712862.2 and XM_009444056.1 provide additional, non-limiting, example sequences of the CD28 transmembrane domain.

As used herein, the term “CD28 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the CD28 costimulatory signaling region sequence shown herein. The example sequences CD28 costimulatory signaling domain are provided in U.S. Pat. No. 5,686,281; Geiger, T. L. et al. (2001) Blood 98: 2364-2371; Hombach, A. et al. (2001) J Immunol 167: 6123-6131; Maher, J. et al. (2002) Nat Biotechnol 20: 70-75; Haynes, N. M. et al. (2002) J Immunol 169: 5780-5786 (2002); Haynes, N. M. et al. (2002) Blood 100: 3155-3163.

As used herein, the term “CD3 zeta signaling domain” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the CD3 zeta signaling domain sequence as shown herein.

As used herein, the term “T-cell receptor”, or “TCR”, is a molecule found on the surface of T cells, or T lymphocytes, that is responsible for recognizing fragments of antigen as peptides bound to major histocompatibility complex (MHC) molecules. The TCR is a heterodimer. There are two types the αβ TCR consisting of an alpha (α) chain and a beta (β) chain (in humans about 95%) and a v5TCR comprising of a gamma (γ) and delta (δ) chain (in humans about 5%). When the TCR engages with antigenic peptide and MHC (peptide/MHC), the T lymphocyte is activated through signal transduction, mediated by associated enzymes, co-receptors, specialized adaptor molecules, and activated or released transcription factors. The variable TCR receptor is part of octomeric complex including additionally the three dimeric signaling modules CD35/£, CD3v/£ and CD247 ζ/ζ or ζ/η.

T cell activation refers to a process in which mature T cells, optionally which express antigen-specific T cell receptors on their surfaces, recognize their cognate antigens and respond by entering the cell cycle, secreting cytokines or lytic enzymes, and initiating the cell-based functions of the immune system. T cell signaling involves a complex network of biochemical interactions, most of which are initiated downstream of the TCR itself, but a few of which can be induced by accessory receptors. In some embodiments, TCR signaling requires close interactions between the T cell and an antigen presenting cell, and as a result involve extensive coordination between TCR signaling networks and both cytoskeletal and membrane dynamic processes. Engagement of the TCR initiates positive and negative cascades that ultimately result in cellular proliferation, differentiation, cytokine production, and/or activation-induced cell death.

In some embodiments, activating T cells by manipulating (such as activating) the TCR signaling is referred to herein as TCR stimulation. In further embodiments, activating T cells by contacting an agent with TCR is referred to herein as TCR stimulation. Accordingly, the agent activating T cells by manipulating (such as activating) the TCR signaling is referred to herein as a TCR stimulator.

In some embodiments, stimulation of TCR is triggered by MHC (major histocompatibility complex) molecules on cells with an antigen. In further embodiments, stimulation of TCR can be trigged by an antigen. In yet further embodiments, stimulation of TCR can be triggered by an antigen presented on an antigen-presenting cell. In some embodiments, T cells can be activated or TCRs can be stimulated by contacting the T cells with a CD3 agonist, such as an anti-CD3 antibody, optionally a monoclonal anti-CD3 antibody. See, for example, Kuhn C and Weiner H L. Immunotherapy. 2016 July; 8(8):889-906.

A “CD3 agonist” refers to an agent capable of modulating CD3 molecules, and in particular inducing a reaction by acting on CD3 molecules expressed on the surfaces of immunocytes resulting in intracellular signal transduction through CD3, and thereby promoting differentiation of the cells. A CD3 agonist may interact with CD3 and modulate the effects of CD3 or it may be a T cell energizing CD3 agonist. Suitable CD3 agonists include but are not limited to agonistic anti-CD3 antibodies. An anti-CD3 antibody agonist can be selected that reduces or reverses release of cytokines including IL-1, IL-6, IL-2, IL-4 and TNF-α. Such selected anti-CD-3 antibodies include antibodies that have been modified by changing the Fc portion of the immunoglobulin molecule by preparing F(ab′)₂ fragments or modifying the Fc portion of the immunoglobulin to render it non-FcR binding. Examples of anti-CD3 antibodies are OKT3 [ATCC CRL-8001; Orthoclone OKT3™ (Muromonab-CD3), Janssen-Ortho Inc.], UCHT1 (B. D. PharMingen), HIT3a (B. D. PharMingen), hOKT3γl(Ala-Ala), CD3 mAb 145 2Cl 1, YTH 12.5.14.2, YTH 12.5, CAMPATH-3 or F(ab′)₂ fragments thereof. See for example, see Published PCT WO04106380; Published US Application No. US20040202657; U.S. Pat. Nos. 6,750,325; 6,706,265; GB No. 2249310A; and Clark et al., European J. Immunol. 1989, 19:381-388; U.S. Pat. No. 5,968,509. In an embodiment, a CD3 agonist is a T cell energizing CD3 agonist.

In some embodiments, TCR stimulation further requires a secondary signal which immune cells rely on to activate an immune response in the presence of an antigen-presenting cell, which is referred to as a co-stimulatory signal. Non-limiting examples of the co-stimulatory signal is an activation of CD28 on a T cell or an activation of Inducible Costimulator (ICOS). Accordingly, in some embodiments, the TCR stimulator as disclosed herein further comprises a CD28 agonist. Additionally or alternatively, the TCR stimulator as disclosed herein further comprises a ICOS agonist.

As used herein, the term “CD28 agonist” refers to an agonistic agent that induces or increases or both induces and increases the biological function or expression or both of CD28. In some embodiments, the CD28 agonist binds directly CD28 and activates the receptor. In some embodiments, the CD28 agonist binds ligands of CD28 (e.g. B7.1 and B7.2) and increases the binding (e.g. affinity) of the ligands to CD28 and/or activation of CD28 by the ligand. In some embodiments, the CD28 agonist indirectly binds CD28 by acting through an intermediary molecule, for example the agonist binds to or modulates a molecule that in turn binds to or modulates CD28. In certain embodiments, the CD28 agonist exhibits one or more desirable functional properties, such as high affinity binding to CD28, e.g., binding to human CD28 with a K_{D} of 10⁻⁷ M, 10⁻⁸ M, 10⁻⁹ M, 10⁻¹⁰ M or less; lack of significant cross-reactivity to other immune-check point proteins, e.g., CTLA-4 and ICOS; the ability to stimulate T cell proliferation; the ability to increase IFN-γ and/or IL-2 secretion; the ability to stimulate antigen-specific memory responses; the ability to stimulate antibody responses and/or the ability to inhibit growth of tumor cells. In some embodiments, the CD28 agonist is the naturally occurring ligand (e.g. B7.1 or B7.2) or a functional derivative or variant thereof which retain the ability to specifically bind to the CD28. Thus, for example the CD28 agonist can be an entire B7.1 or B7.2, soluble B7.1 or B7.2 or fragments thereof and fusion proteins comprising a functionally active portion of B7.1 or B7.2 covalently linked to a second protein domain, that binds to and activates CD28, such as disclosed for example in U.S. Patent Application Publication No. 20030232323; and International Application Publication No. WO1995003408, which are hereby incorporated by reference in their entirety. In some embodiments, the CD28 agonist is an antibody. In some embodiments, the CD28 agonist is an anti-CD28 antibody. The anti-CD28 antibody can be a superagonistic anti-CD28 antibody or a conventional anti-CD28 antibody. In further embodiments, the CD28 agonist is a monoclonal anti-CD28 antibody.

An agonist may agonize the expression or the activity or both the expression and the activity of the molecule, such as CD3, CD28 or ICOS. An activity is “agonized” if the activity is increased by at least about 10%, e.g., 50%, in the presence, relative to the absence of an agonist. An expression is “agonized” if the expression is increased by at least about 10%, e.g., 50%, in the presence, relative to the absence of an agonist.

As used herein, an “agonist” of a molecule, such as CD3, CD28 or ICOS, refers to an agonistic agent that induces or increases or both induces and increases the biological function or expression or both of molecule. In some embodiments, the agonist binds directly to the molecule and activates the molecule. In some embodiments, the agonist binds ligands of the molecule and increases the binding (e.g. affinity) of the ligands to the molecule and/or activation of the molecule by the ligand. In some embodiments, the agonist indirectly binds the molecule by acting through an intermediary molecule, for example the agonist binds to or modulates a molecule that in turn binds to or modulates the molecule. In certain embodiments, the agonist exhibits one or more desirable functional properties, such as high affinity binding to the molecule, e.g., binding to the molecule with a K_{D} of 10⁻⁷ M, 10⁻⁸ M, 10⁻⁹ M, 10⁻¹⁰ M or less; lack of significant cross-reactivity to other immune-check point proteins; the ability to stimulate T cell proliferation; the ability to increase IFN-γ and/or IL-2 secretion; the ability to stimulate antigen-specific memory responses; the ability to stimulate antibody responses and/or the ability to inhibit growth of tumor cells. In some embodiments, the agonist is the naturally occurring ligand or a functional derivative or variant thereof which retain the ability to specifically bind to the molecule. Thus, for example the agonist can be an entire ligand, soluble ligand or fragments thereof and fusion proteins comprising a functionally active portion of the ligand covalently linked to a second protein domain, that binds to and activates the molecule. In some embodiments, the agonist is an antibody. In some embodiments, the agonist is an antibody specifically recognizing and binding the molecule. The antibody can be a superagonistic antibody or a conventional antibody. In further embodiments, the agonist is a monoclonal antibody.

The TCR a and β chains are both composed of a variable part and a constant part. The diversity is based mainly on genetic recombination of the DNA encoded segments in individual somatic T cells—either somatic V(D)J recombination using RAG1 and RAG2 recombinases or gene conversion using cytidine deaminases (AID). V(D)J recombination occurs in the primary lymphoid organs (bone marrow for B cells and thymus for T cells) and in a nearly random fashion rearranges variable (V), joining (J), and in some cases, diversity (D) gene segments. The process ultimately results in novel amino acid sequences in the antigen-binding regions of Igs and TCRs that allow for the recognition of antigens.

“Vbeta” or “vβ” relates to the variable portion of the beta chain of the TCR composed of the gene products of the variable (V), joining (J), and in some cases, diversity (D) gene segments.

A “composition” typically intends a combination of the active agent, e.g., an cell or an engineered immune cell, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

As used herein, the term “excipient” refers to an inert substance which is commonly used as a diluent, vehicle, preservative, binder, or stabilizing agent for an agent, such as a cell as disclosed herein and includes, but is not limited to, proteins (e.g., serum albumin, etc.), amino acids (e.g., aspartic acid, glutamic acid, lysine, arginine, glycine, histidine, alanine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). Also see Remington's Pharmaceutical Sciences (by Joseph P. Remington, 18th ed., Mack Publishing Co., Easton, Pa.) and Handbook of Pharmaceutical Excipients (by Raymond C. Rowe, 5th ed., APhA Publications, Washington, D.C.) which are hereby incorporated in its entirety. Preferably, the excipients impart a beneficial physical property to the formulation, such as increased stability, increased solubility and decreased viscosity.

A “preservative” is a natural or synthetic chemical that is added to products such as foods, pharmaceuticals, paints, biological samples, wood, etc. to prevent decomposition by microbial growth or by undesirable chemical changes. Preservative additives can be used alone or in conjunction with other methods of preservation. Preservatives may be antimicrobial preservatives, which inhibit the growth of bacteria and fungi, or antioxidants such as oxygen absorbers, which inhibit the oxidation of constituents. Common antimicrobial preservatives include, benzalkonium chloride, benzoic acid, cholorohexidine, glycerin, phenol, potassium sorbate, thimerosal, sulfites (sulfur dioxide, sodium bisulfite, potassium hydrogen sulfite, etc.) and disodium EDTA. Other preservatives include those commonly used in patenteral proteins such as benzyl alcohol, phenol, m-cresol, chlorobutanol or methylparaben.

The term “stabilizer” refers to pharmaceutically acceptable stabilizers, like for example but not limited to amino acids and sugars as well as commercially available dextrans of any kind and molecular weight as known in the art.

As used herein, the term “allogenic” refers to the material (e.g., a cell, a tissue, an organ, etc.) of an entity which is from another entity having the same species but genetically different. Since an allogenic entity is genetically different, the allogenic entity may elicit an immune reaction in an entity (recipient) to which the allo-entity is administered.

As used herein, the term “heterologous” refers to a material (e.g., a cell, a tissue, an organ, etc.) which is from a different species entity. Therefore, for example, when a human is a recipient, a porcine-derived graft is called a heterologous graft.

The term “autologous” refers to any material derived from the same subject to whom it is later to be re-introduced into the subject.

As used herein, the term “antihistamine” refers to a moiety that inhibits reduces effects mediated by histamine. By way of example, moieties having negative modulation of histamine receptors as their main therapeutic effect (i.e., antagonists at the histamine H1 receptor) are antihistamines. For example, chlorpheniramine, which may also have anticholinergic activity, is considered an antihistamine. Antihistamine includes, for example, first generation antihistamines such as ethylenediamines, ethanolamines, alkylamines, piperazines and tricyclics. Antihistamines further include second generation antihistamines such as acrivastine, astemizole, cetirizine, loratadine, mizolastine, desloratidine and fexofenadine.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone), dexamethasone triamcinolone, budesonide, and betamethasone.

As used herein, a “mast cell stabilizer” refers to an agent that inhibits degranulation and/or the release of pro-inflammatory and vasoactive mediators from mast cells. Mast cell stabilizers include, but are not limited to, cromolyn, dihydropyridines such as nicardipine and nifedipine, lodoxamide, nedocromil, barnidipine, YC-114, elgodipine, niguldipine, ketotifen, methylxanthines, quercetin, and pharmaceutically salts thereof. In some embodiments, the mast cell stabilizer is a pharmaceutically acceptable salt of cromolyn, such as cromolyn sodium, cromolyn lysinate, ammonium cromonglycate, and magnesium cromoglycate. In some embodiments, mast cell stabilizers include but are not limited to compounds disclosed in U.S. Pat. Nos. 6,207,684; 4,634,699; 6,207,684; 4,871,865; 4,923,892; 6,225,327; 7,060,827; 8,470,805; 5,618,842; 5,552,436; 5,576,346; 8,252,807; 8,088,935; 8,617,517; 4,268,519; 4,189,571; 3,790,580; 3,720,690; 3,777,033; 4,067,992; 4,152,448; 3,419,578; 4,847,286; 3,683,320; and 4,362,742; U.S. Patent Application Publication Nos. 2011/112183 and 2014/140927; European Patent Nos. 2391618; 0163683; 0413583; and 0304802; International Patent Application Nos. WO2010/042504; WO85/02541; WO2014/115098; WO2005/063732; WO2009/131695; and WO2010/088455; all of which are incorporated by reference. Mast cell stabilizers, including cromolyn and pharmaceutically acceptable salts, prodrugs, and adducts thereof, may be prepared by methods known in the art.

A “leukotriene modifier”, which is also referred to as a leukotriene receptor antagonist, helps prevent breathing problems associated with allergies, asthma and chronic obstructive pulmonary disease. Examples include, but are not limited to, montelukast, zafirlukast and zileuton.

As used herein, the terms “variant” refers to an equivalent having a native polypeptide sequence and structure with one or more amino acid additions, substitutions (generally conservative in nature) or deletions, so long as the modifications do not destroy biological activity and which are substantially identical to the reference polypeptide. Variants generally include substitutions that are conservative in nature, i.e., those substitutions that take place within a family of amino acids that are related in their side chains. Specifically, amino acids are generally divided into four families: (1) acidic: aspartate and glutamate; (2) basic: lysine, arginine, histidine; (3) non-polar: alanine, valine, leucine, isoleucine, proline, phenylalanine, methionine, tryptophan; and (4) uncharged polar: glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest can include up to about 5-10 conservative or non-conservative amino acid substitutions, or even up to about 15-25 conservative or non-conservative amino acid substitutions, or any integer between 5-25, so long as the desired function of the polypeptide remains intact. One of skill in the art can readily determine regions of the polypeptide of interest that can tolerate change by reference to Hopp/Woods and Kyte-Doolittle plots, well known in the art.

In some embodiments, a variant can be a derivative. By “derivative” is intended any suitable modification of the native polypeptide of interest, of a fragment of the native polypeptide, or of their respective variant, such as glycosylation, phosphorylation, polymer conjugation (such as with polyethylene glycol), or other addition of foreign moieties, so long as the desired biological activity of the native polypeptide is substantially retained. Methods for making polypeptide fragments, variants, and derivatives are generally available in the art.

As used herein, a polypeptide as referred to herein also intend to comprise, or alternatively consist essentially of, or yet further consist of an equivalent of the polypeptide, such as a variant or a fragment of the polypeptide.

The term equivalent and biological equivalent are used interchangeably, for example when referring to a protein or polypeptide as a reference. In some embodiments, an equivalent protein or polypeptide is one having at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to the reference protein or polypeptide. In some embodiments, an equivalent protein or polypeptide has at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to a polypeptide or protein as disclosed herein. In addition or alternatively, a polypeptide or an equivalent thereof have the same or substantially similar biological activity (also referred to as function). In further embodiments, the equivalent is a functional protein that optionally can be identified through one or more assays described herein.

By “fragment” is intended a molecule consisting of only a part of the intact full-length sequence and structure. The fragment can include a C-terminal deletion, an N-terminal deletion, an internal deletion of the native polypeptide, or any combination thereof. Active fragments of a particular protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, or any integer between 5 amino acids and the full-length sequence, provided that the fragment in question substantially retains biological activity.

In some embodiments, the term “engineered” refers to comprising at least one modification not normally found in a naturally occurring counterpart, wild-type or a parent. Such as an engineered T cell can comprise a chimeric antigen receptor (CAR) that is not naturally occurring. In some embodiments, the term “engineered” is used interchangeably with “recombinant” refers to being synthetized by human.

The term “isolated” as used herein refers to molecules, biologicals, cellular materials, cells or biological samples being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. In some embodiments, the term “isolated” is used herein to refer to cells or tissues that are isolated from other cells or tissues and is meant to encompass both cultured and engineered cells or tissues.

As used herein, the term “isolated cell” generally refers to a cell that is substantially separated from other cells of a tissue.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, a “cancer” is a disease state characterized by the presence in a subject of cells demonstrating abnormal uncontrolled replication and in some aspects, the term may be used interchangeably with the term “tumor.” The term “cancer or tumor antigen” refers to an antigen known to be associated and expressed on the surface with a cancer cell or tumor cell or tissue, and the term “cancer or tumor targeting antibody” refers to an antibody that targets such an antigen.

The terms or “acceptable,” “effective,” or “sufficient” when used to describe the selection of any components, ranges, dose forms, etc. as disclosed herein intend that said component, range, dose form, etc. is suitable for the disclosed purpose.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

“Therapeutically effective amount” of a drug or an agent refers to an amount of the drug or the agent that is an amount sufficient to obtain a pharmacological response; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

In some embodiments, a subject described herein is suspected of or suffering from a pathogenic infection also suffers from an inflammation of the skin, airway mucosa, or a combination thereof. In some instances, the pathogenic infection is a viral infection. In some cases, the virus that induces the infection is a member of the coronaviruses.

Coronaviruses is a family of single-stranded, positive-strand RNA viruses characterized with crown-like spikes on their surface. The coronaviruses belong to the Coronaviridae family, Nidovirales order. There are four sub-groupings or categories of CoVs, alpha, beta, gamma, and delta. The CoVs are the largest known RNA viruses, comprising 16 non-structural proteins and 4 structural proteins which include spike (S) protein, envelope (E) protein, membrane (M) protein, and nucleocapsid (N) protein.

There are seven species of coronaviruses that are known to cause respiratory and intestinal infections in humans. The seven species are 229E (or α-type HCoV-229E), NL63 (or α-type HCoV-NL63), OC43 (or β-type HCoV-OC43), HKU1 (or β-type HCoV-HKU1), MERS-CoV (the β-type HCoV that causes Middle East Respiratory Syndrome or MERS), SARS-CoV (the β-type HCoV that causes severe acute respiratory syndrome or SARS), and SARS-CoV2 (the β-type HCoV that causes the coronavirus disease of 2019, COVID-19, or 2019-nCoV).

In some embodiments, the CoVs are also classified based on their pathogenicity. In some instances, the mild pathogenic CoVs include HCoV-229E, HCoV-OC43, HCoV-NL63, and HCoV-HKU1. In some instances, the highly pathogenic CoVs include SARS-CoV, MERS-CoV, and SARS-CoV2. In some cases, the mild pathogens infect the upper respiratory tract and causes seasonal, mild to moderate cold-like respiratory diseases in the subject. In some cases, the highly pathogenic CoVs infect the lower respiratory tract and cause severe pneumonia, leading, in some cases, to fatal acute lung injury (ALI) and/or acute respiratory distress syndrome (ARDS).

In some embodiments, the virus that induces the infection is an influenza virus, cytomegalovirus, Epstein-Barr virus, variola virus, Ebola, dengue, Measles virus, mumps virus, or rubella virus. In some instances, the influenza virus is influenza A virus.

In some embodiments, the pathogenic infection is induced by a bacterium, optionally a Gram-positive bacterium or a Gram-negative bacterium. In some instances, the bacterium comprises a members of the Streptococcus family and forms group A streptococcus (GAS). GAS is a plurality of Gram-positive, beta-hemolytic coccus in chains and causes, e.g., strep throat, skin and soft tissue infections such as impetigo and cellulitis, or toxic shock syndrome (TSS). In some embodiments, the GAS comprises Streptococcus pyogenes or Streptococcus dysgalactiae.

In some instances, the bacterium is Francisella tularensis, Corynebacterium diphtheria, Legionella pneumophila, Streptococcus pneumoniae, Mycobacterium tuberculosis, Bordetella pertussis, Bacillu anthracis, Chlamydia psittaci, Coxiella burnetti, Francisella tularensis, or from the genus Brucella.

In some embodiments, the pathogenic infection is induced by a protozoan. In some instances, the protozoans include, but are not limited to, Plasmodium falciparum and Entamoeba histolytica.

In some embodiments, the pathogenic infection is induced by a fungus. In some instances, the fungi include, but are not limited to, the genus of Aspergillus, Candida, or Cryptococcus; a member of the Pneumocystis species; a member of Dermatophytosis (also known as ringworm); or a member of Basidiomycota.

In some embodiments, the pathogenic infection is induced by a parasite. In some instances, the parasite include a nematode or a trematode. In some cases, the parasite is Echinococcus granulosus, Dirofilaria immitis, Paragonimus westermani, Ascaris lumbricoides, Ancylostoma duodenale, Toxocara canis, Schistosoma mansoni, Schistosoma haematobium, Schistosoma japonicum, Strongyloides stercoralis, Wuchereria bancrofti, or Brugia malayi.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

In some embodiments, the composition disclosed herein (e.g., pharmaceutical composition and formulations) are administered to a subject by multiple administration routes, including but not limited to, parenteral, oral, buccal, rectal, sublingual, or transdermal administration routes. In some cases, parenteral administration comprises intravenous, subcutaneous, intramuscular, intracerebral, intranasal, intra-arterial, intra-articular, intradermal, intravitreal, intraosseous infusion, intraperitoneal, or intratechal administration. In some instances, the composition (e.g., pharmaceutical composition) is formulated for local administration. In other instances, the composition (e.g., pharmaceutical composition) is formulated for systemic administration.

In some embodiments, the composition (e.g., pharmaceutical composition or formulations) include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the composition (e.g., pharmaceutical composition or formulations) include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pa. 1975, Liberman, H. A. and Lachman, L., Eds., Pharmaceutical Dosage Forms, Marcel Decker, New York, N.Y., 1980, and Pharmaceutical Dosage Forms and O g Delivery Systems, Seventh Ed. (Lippincott Williams & Wilkins 1999).

In some instances, the composition (e.g., pharmaceutical composition or formulations) further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the composition (e.g., pharmaceutical composition or formulations) includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the composition (e.g., pharmaceutical composition or formulations) include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the composition (e.g., pharmaceutical composition or formulations) further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®, dibasic calcium phosphate, dicalcium phosphate dihydrate, tricalcium phosphate, calcium phosphate, anhydrous lactose, spray-dried lactose, pregelatinized starch, compressible sugar, such as Di-Pac® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the composition (e.g., pharmaceutical composition or formulations) include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel®PH102, Avicel® PH105, Elcema® P100, Emcocel®, Vivacel®, MingTia®, and SolkaFloc®, methylcellulose, croscarmellose, or a cross-linked cellulose, such as cross-linked sodium carboxymethylcellulose (Ac-Di-Sol), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the composition (e.g., pharmaceutical composition or formulations) include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotee), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™ Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or Tween® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., PLUIRONIC® (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

As it would be understood by one of skill in the art, any embodiments, instances, aspects, examples, or cases can be combined or substituted with any other embodiments, instances, aspects, examples, or cases as disclosed herein, no matter where the embodiments, instances, aspects, examples or cases are provided in this disclosure.

### Methods of Detection

Disclosed herein, in certain embodiments, are methods of detecting the presence of an antigen-specific T cell population in a biological sample that exerts a beneficial effect to a subject suffering an allergy. In some embodiments, the antigen-specific T cell population dampens or decreases an inflammatory response in the subject, such as in the lung. In some cases, the method comprises, or alternatively consists essentially of, or yet further consists of detecting the presence of an antigen-specific T cell characterized with an elevated expression level of an interferon response gene profile in a biological sample. In some embodiments, the method comprises, or alternatively consists essentially of, or yet further consists of: (a) generating a gene expression profile of at least one T cell from each of a population of CD154-enriched T cells, a population of CD137-enriched T cells, or a population of CD4-enriched T cells obtained from a biological sample by a sequencing method; and (b) analyzing the gene expression profile to detect the presence of an antigen-specific T cell that express an elevated level of an interferon response gene profile in the biological sample. In some embodiments, the method comprises, or alternatively consists essentially of, or yet further consists of: (a) generating a gene expression profile of at least one T cell from one or more of a population of CD154-enriched T cells, a population of CD137-enriched T cells, or a population of CD4-enriched T cells obtained from a biological sample by a sequencing method; and (b) analyzing the gene expression profile to detect the presence of the antigen-specific T cell that expresses the elevated level of an interferon response gene profile in the biological sample.

Additionally or alternatively, the at least one T cell of step (a) can be from any T cell populations, such as CD3 positive T cells (i.e., T cells expressing CD3, which is also referred to herein as CD3-enriched T cells), CD4 positive T cells (i.e., T cells expressing CD4, which is also referred to herein as CD4-enriched T cells), CD4 positive helper T cells (i.e., helper T cells expressing CD4, which is also referred to herein as CD4-enriched helper T cells or T_{H} cells), CD8 positive T cells (i.e., T cells expressing CD8, which is also referred to herein as CD8-enriched T cells), CD154 positive T cells (i.e., T cells expressing CD154, which is also referred to herein as CD154-enriched T cells), CD137 positive T cells (i.e., T cells expressing CD137, which is also referred to herein as CD137-enriched T cells), CD154 negative CD137 positive T cells (i.e., T cells not expressing CD154 but expressing CD137), CD154 negative CD137 negative T cells (i.e., T cells not expressing CD154 or CD137), or a regulatory T cells (T_{REG} cells).

In some embodiments, the antigen-specific T cell as used herein can be substituted with a T cell that is not antigen-specific, i.e., not specifically recognizing or binding to the antigen. In further embodiments, the method reciting the non-antigen-specific T cell further comprises a step of contacting the non-antigen-specific T cell with the antigen. In yet further embodiments, the method further comprises isolating or enriching or both isolating and enriching antigen-specific T cells after contacting the non-antigen-specific T cell with the antigen. Non-limiting example of the isolating or enriching method include a flow cytometry or a tetramer assay.

In some embodiments, the antigen that is specifically recognized and bound to by the antigen-specific T cell can cause an allergy, and accordingly such antigen is referred to herein as an allergen. In further embodiments, the allergen is a house dust mite (HDM) or an antigen thereon.

In certain embodiments, provided are methods of detecting the presence of an antigen-specific T cell population in a biological sample that exerts a beneficial effect to a subject infected with a pathogen, such as a virus. In some embodiments, the pathogen, such as a virus, infects the subject's lung. In some embodiments, the antigen-specific T cell population dampens or decreases an inflammatory response induced by the pathogen in the subject, such as in the lung. In some cases, the method comprises, or alternatively consists essentially of, or yet further consists of detecting the presence of an antigen-specific T cell characterized with an elevated expression level of an interferon response gene profile in a biological sample. In some embodiments, the method comprises, or alternatively consists of, or yet further consists of: (a) generating a gene expression profile of at least one T cell from one or more of a population of CD154-enriched T cells, a population of CD137-enriched T cells, a population of CD4-enriched T cells obtained from a biological sample by a sequencing method; and (b) analyzing the gene expression profile to detect the presence of the antigen-specific T cell that expresses the elevated level of the interferon response gene profile in the biological sample.

In some embodiments, also disclosed herein are methods of detecting the presence of a house dust mite (HDM)-reactive T cell population in a biological sample that exerts a beneficial effect to a subject suffering from a HDM-induced allergy. In some instances, the HDM-reactive T cell population dampens or decreases an inflammatory response in the subject, such as in the lung. In some instances, the method comprises, or alternatively consists of, or yet further consists of detecting the presence of a HDM-reactive T cell characterized with an elevated expression level of an interferon response gene profile in a biological sample. In some cases, the method comprises, or alternatively consists of, or yet further consists of (a) generating a gene expression profile of at least one T cell from one or more (such as each) of a population of CD154-enriched T cells, a population of CD137-enriched T cells, or a population of CD4-enriched T cells obtained from a biological sample by a sequencing method; and (b) analyzing the gene expression profile to detect the presence of the HDM-reactive T cell that expresses an elevated level of an interferon response gene profile in the biological sample.

In some embodiments, detection of the presence of the T cell that expresses the elevated level of the interferon response gene profile indicates the subject from whom the biological sample was obtained does not have an inflammation or is at a low risk of developing the inflammation. Additionally or alternatively, more or a higher percentage of T cells in the biological sample expressing the elevated level of the interferon response gene profile indicates the subject from whom the biological sample was obtained does not have an inflammation or is at a low risk of developing the inflammation. Accordingly, less or a lower percentage of T cells in the biological sample expressing the elevated level of the interferon response gene profile indicates the subject from whom the biological sample was obtained has an inflammation or is at a high risk of developing the inflammation.

In some embodiments, the inflammation (which is also referred to herein as the inflammation response) is an undesired inflammation leading to damaging the subject's normal cells, tissues or organs or causing itching, pain or other unwanted symptom. In further embodiments, the inflammation is an allergic inflammation. In yet further embodiments, the inflammation is an allergic airway inflammation, such as an allergic lung inflammation. Additionally or alternatively, the inflammation is a type II inflammation, which is also referred to herein as a T_{H}2 inflammation. The type II inflammation can occur in response to atopic disease, including but not limited to asthma and certain parasitic infections. It leads to a humoral immune response. In some embodiments, the inflammation comprises, or consists essentially of, or yet further consists of a humoral immune response. In some embodiments, the inflammation comprises, or consists essentially of, or yet further consists of IgG release. In some embodiments, the inflammation comprises, or consists essentially of, or yet further consists of a cytokine storm. In some embodiments, the inflammation is caused by a pathogen, such as SARS-COV-2 or others as disclosed herein. In some embodiments, the inflammation is a symptom of or is caused by an allergy.

In some embodiments, the interferon response gene profile comprises, or consists essentially of, or yet further consists of an elevated expression level of at least one of TNFSF10, CXCL10, IFI6, MX1, ISG15, ISG20, OAS1, OAS3, IFIT1, IFIT3, IFI44L, SAMDL9, or any combination thereof. In some instances, the interferon response gene profile comprises, or consists essentially of, or yet further consists of an elevated expression level of at least one of TNFSF10, CXCL10, or a combination thereof. In some instances, the interferon response gene profile comprises, or consists essentially of, or yet further consists of an elevated expression level of at least one of IFI6, MX1, ISG15, ISG20, OAS1, OAS3, IFIT1, IFIT3, IFITM1, IFI44L, SAMDL9, or any combination thereof. In some instances, the interferon response gene profile comprises, or consists essentially of, or yet further consists of an elevated expression level of TNFSF10, i.e., expressing TRAIL at an elevated level In some instances, the interferon response gene profile comprises, or consists essentially of, or yet further consists of an elevated expression level of CXCL10.

In some embodiments, the antigen-specific T cell is an antigen-specific T_{H}2 cell that express an elevated expression level of an interferon response gene profile as disclosed herein. In some embodiments, the antigen-specific T cell is an antigen-specific T_{H} cell that express an elevated expression level of an interferon response gene profile as disclosed herein. Additionally or alternatively, the antigen-specific T cell is an antigen-specific T regulatory (T_{reg}) cell that express an elevated expression level of an interferon response gene profile as disclosed herein.

In some instances, the antigen-specific T_{H}2 cell comprises, or consists essentially of, or yet further consists of an elevated expression level of at least one of TNFSF10, CXCL10, IFI6, MX1, ISG15, ISG20, OAS1, OAS3, IFIT1, IFIT3, IFI44L, or any combination thereof. In some cases, the antigen-specific T_{H}2 cell comprises, or consists essentially of, or yet further consists of an elevated expression level of at least one of IFI6, MX1, ISG15, ISG20, OAS1, OAS3, IFIT1, IFIT3, IFITM1, IFI44L, or any combination thereof. In some cases, the antigen-specific T_{H}2 cell comprises, or consists essentially of, or yet further consists of an elevated expression level of at least one of TNFSF10, CXCL10, or a combination thereof. In some cases, the antigen-specific T_{H}2 cell comprises, or consists essentially of, or yet further consists of an elevated expression level of TNFSF10. In some cases, the antigen-specific T_{H}2 cell comprises, or consists essentially of, or yet further consists of an elevated expression level of CXCL10.

In some instances, the antigen-specific T_{H} cell comprises, or consists essentially of, or yet further consists of an elevated expression level of at least one of TNFSF10, CXCL10, IFI6, MX1, ISG15, ISG20, OAS1, OAS3, IFIT1, IFIT3, IFITM1, IFI44L, or any combination thereof. In some cases, the antigen-specific T_{H} cell comprises, or consists essentially of, or yet further consists of an elevated expression level of at least one of IFI6, MX1, ISG15, ISG20, OAS1, OAS3, IFIT1, IFIT3, IFITM1, IFI44L, or any combination thereof. In some cases, the antigen-specific T_{H} cell comprises, or consists essentially of, or yet further consists of an elevated expression level of at least one of TNFSF10, CXCL10, or a combination thereof. In some cases, the antigen-specific T_{H} cell comprises, or consists essentially of, or yet further consists of an elevated expression level of TNFSF10. In some cases, the antigen-specific T_{H} cell comprises, or consists essentially of, or yet further consists of an elevated expression level of CXCL10.

In some instances, the antigen-specific T_{reg} cell comprises, or consists essentially of, or yet further consists of an elevated expression level of at least one of TNFSF10, ISG15, IFI6, MX1, IFIT3, SAMDL9, OAS1, or any combination thereof. In some cases, the antigen-specific T_{reg} cell comprises, or consists essentially of, or yet further consists of an elevated expression level of TNFSF10. In some cases, the antigen-specific T_{reg} cell comprises, or consists essentially of, or yet further consists of an elevated expression level of at least one of ISG15, IFI6, MX1, IFIT3, SAMDL9, OAS1, or any combination thereof.

In some embodiments, the antigen-specific T cell is a house dust mite (HDM)-reactive T cell. In some instances, the HDM-reactive T cell is characterized with an interferon response gene profile comprising, or consists essentially of, or yet further consists of an elevated expression level of at least one of TNFSF10, CXCL10, IFI6, MX1, ISG15, ISG20, OAS1, OAS3, IFIT1, IFIT3, IFITM1, IFI44L, SAMDL9, or any combination thereof.

In some instances, the HDM-reactive T cell is a HDM-reactive T_{H}2 cell that express an elevated expression level of an interferon response gene profile (T_{H}IFNR). In some instances, the HDM-reactive T cell is a HDM-reactive T_{H} cell that express an elevated expression level of an interferon response gene profile (T_{H}IFNR). In some instances, the T_{H}IFNR comprises, or consists essentially of, or yet further consists of an elevated expression level of at least one of TNFSF10, CXCL10, IFI6, MX1, ISG15, ISG20, OAS1, OAS3, IFIT1, IFIT3, IFITM1, IFI44L, or any combination thereof. In some instances, the T_{H}IFNR comprises an elevated expression level of at least one of IFI6, MX1, ISG15, ISG20, OAS1, OAS3, IFIT1, IFIT3, IFITM1, IFI44L, or any combination thereof. In some instances, the T_{H}IFNR comprises an elevated expression level of at least one of TNFSF10, CXCL10, or a combination thereof. In some instances, the T_{H}IFNR comprises an elevated expression level of TNFSF10. In some instances, the T_{H}IFNR comprises an elevated expression level of CXCL10.

In some embodiments, the HDM-reactive T cell is a HDM-reactive T regulatory (T_{reg}) cell that express an elevated expression level of an interferon response gene profile (T_{reg}IFNR). In some instances, the T_{reg}IFNR comprises an elevated expression level of at least one of TNFSF10, ISG15, IFI6, MX1, IFIT3, SAMDL9, OAS1, or any combination thereof. In some instances, the T_{reg}IFNR comprises an elevated expression level of TNFSF10. In some instances, the T_{reg}IFNR comprises an elevated expression level of at least one of ISG15, IFI6, MX1, IFIT3, SAMDL9, OAS1, or a combination thereof.

In some embodiments, the interferon response gene profile further comprises an elevated expression level of at least one of XCL2, SEMATA, CXCR3, FASLG, IFNG, PRF1, KLRG1, XCL1, CD226, NFATC1, or any combination thereof.

In some embodiments, the interferon response gene profile comprises an elevated expression level of a gene selected from T_{H}IFNR group of Table 1.

In some embodiments, the interferon response gene profile comprises an elevated expression level of a gene selected from T_{H}1 group of Table 1.

In some embodiments, the elevated expression level of the interferon response gene profile is compared to a control. In some instances, the control is an interferon response gene profile of a CD154 positive T cell that has not been stimulated by an antigen, such as an allergen (e.g., HDM). In some instances, the control is an interferon response gene profile of a CD137 positive T cell that has not been stimulated by an antigen, such as an allergen (e.g., HDM). In some instances, the control is an interferon response gene profile of a CD154 negative CD137 positive T cell that has not been stimulated by an antigen, such as an allergen (e.g., HDM). In some embodiments, the control is an interferon response gene profile of a CD4 positive T cell that has not been stimulated with the antigen, such as an allergen (e.g., HDM).

In some embodiments, the antigen is the one specifically recognized and bound to by the antigen-specific T cells.

In some embodiments, the elevated expression level of the interferon response gene profile is compared to a control. In some instances, the control is an interferon response gene profile of a CD154 positive T cell. In some instances, the control is an interferon response gene profile of a CD137 positive T cell. In some instances, the control is an interferon response gene profile of a CD154 negative CD137 positive T cell. In some embodiments, the control is an interferon response gene profile of a CD4 positive T cell.

In some embodiments, the control is the expression level of the interferon response gene profile of one or more of a T_{H}1 cell, a T_{H}2 cell, a T_{H}17, a T_{H}ACT1, a T_{H}ACT2, a T_{H}ACT3, TREGACT1, TREGACT2, an antigen-specific T cell, a non-antigen-specific T cell, an activated T cell, an inactivated T cell, another T cell as disclosed herein, or an average thereof.

In some embodiments, the control has not been stimulated by an antigen, such as an allergen (e.g., HDM). In some embodiments, the antigen is the one specifically recognized and bound to by the antigen-specific T cells.

In some cases, the expression level of the interferon response gene profile compared to the control is elevated by about 1-fold, 2-fold, 3-fold, 4-fold, 5-fold, 6-fold, 7-fold, 8-fold, 9-fold, 10-fold, 20-fold, 30-fold, 50-fold, 100-fold, or higher, for example of the control. In some cases, the expression level of the interferon response gene profile compared to the control is elevated by about 10%, 20%, 30%, 40%, 50%, 60%, 70%, 80%, 90%, 100%, 200%, 500%, or more, for example of the control.

In some embodiments, the method further comprises harvesting an antigen-specific T cell (e.g., a HDM-reactive T cell) that express an elevated level of an interferon response gene profile. In some embodiments, the method further comprises enriching or isolating or both enriching and isolating the antigen-specific T cell that express an elevated level of an interferon response gene profile as disclosed herein. In some embodiments, the method further comprises enriching or isolating or both enriching and isolating the antigen-specific T cell identified in step (b) that expresses an elevated level of an interferon response gene profile as disclosed herein.

In some embodiments, provided is a composition comprising, or consisting essentially of, or yet further consisting of a plurality of antigen-specific T cells (e.g., HDM-reactive T cells) that is further obtained and can be detectable using the method described for detecting the presence of an antigen-specific T cell (e.g., a HDM-reactive T cell) characterized with an elevated expression level of an interferon response gene profile in a biological sample and subsequent harvesting of the antigen-specific T cells (e.g., the HDM-reactive T cells). In some embodiments, provided is a composition comprising, or consisting essentially of, or yet further consisting of a plurality of T cells as detected, isolated or enriched as disclosed herein. In further embodiments, the T cells comprises, or consists essentially of, or yet further consists of T_{H}IFNR cells or T_{REG}IFNR or both. In yet further embodiments, the T cells are substantially free of T_{H}2 cells. In some cases, the composition further comprises a carrier, excipient, stabilizer, or preservative.

### Methods of Generation

In some embodiments, also disclosed herein is a method of detecting the presence or an interleukin 9 (IL-9)-expressing antigen-specific T cell in a biological sample. In some instances, the IL-9-expressing antigen-specific T cell is associated with a negative prognosis or a worsening of the allergic response or both in a subject exposed to an allergen or suffering from an allergy. In some instances, the IL-9-expressing antigen-specific T cell is associated with a negative prognosis and/or a worsening infection in a subject exposed to a pathogen, such as a virus. In some cases, the method comprises, or alternatively consists essentially of, or yet further consists of (a) generating a gene expression profile for at least one T cell from one or more (such as each) of a population of CD154-enriched T cells, a population of CD137-enriched T cells, or a population of CD4-enriched T cells obtained from a biological sample by a sequencing method; and (b) analyzing the gene expression profile to detect the presence of the IL-9-expressing antigen-specific T cell in the biological sample.

In some embodiments, the IL-9-expressing antigen-specific T cell is an IL-9-expressing antigen (such as HDM)-reactive T cell. In some instances, the IL-9-expressing antigen (such as HDM)-reactive T cell is associated with a negative prognosis or a worsening of the allergic response or both in a subject exposed to HDM or suffering from a HDM-induced allergy. In some cases, the method comprises, or alternatively consists essentially of, or yet further consists of (a) generating a gene expression profile for at least one T cell from one or more (such as each) of a population of CD154-enriched T cells, a population of CD137-enriched T cells, or a population of CD4-enriched T cells obtained from a biological sample by a sequencing method; and (b) analyzing the gene expression profile to detect the presence of the IL-9-expressing antigen (such as HDM)-reactive T cell in the biological sample.

### Cells and Compositions

In some embodiments, a method as disclosed herein further comprises isolating or enriching or both isolating and enriching the detected T cells. In some embodiments, also provided are the detected or isolated or enriched T cells as disclosed herein. In further embodiments, also provided are the T_{H}IFNR cells or the T_{REG}IFNR cells or both. In yet further embodiments, a composition is provided comprising, or consisting essentially of, or yet further consisting of the detected or isolated or enriched T cells. In some embodiments, a composition is provided comprising, or consisting essentially of, or yet further consisting of the T_{H}IFNR cells or the T_{REG}IFNR cells or both. In further embodiments, the composition further comprises one or more of a carrier, excipient, stabilizer or preservative. In some embodiments, the composition can be stored, such as cryopreserved, or transported or both stored and transported. In further embodiments, the provided cells and compositions can be used in treating a subject in need thereof. The compositions are useful diagnostically and therapeutically as described herein. In one aspect the compositions are useful to treat humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). A mammal can be any age or at any stage of development (e.g., an adult, teen, child, infant, or a mammal in utero). A mammal can be male or female. In some embodiments, a human has or is suspected of having an inflammatory condition such as asthma, or a cancer or a neoplastic disorder. The method can be used as a first line, second line, third line, fourth line or fifth line therapy, and combined with other suitable therapies, e.g., surgical recession. In another aspect, the cancer is characterized as being hyporesponsive.

In some embodiments, the analyzing comprises, or alternatively consists essentially of, or yet further consists of detecting the expression or expression level of gene IL-5, IL-9, ILIRL1, ZEB2, MAF, MAP3K8, GZMB, MT-ND5, DUSP6, SEC61G, DNAJC3, AHI1, CDK2AP2, FKBP11, FKBP1A, KDELR2, CD109, or SEC61B, or any combination thereof.

In some embodiments, the analyzing further comprises detecting the expression or expression level of gene PPARG, TGFBR3, IL-13, IL-4, IL-31, IL-3, IL-33R, ICOS, IL-21, PLA2G16, GATA3, IL17RB, GADD45G, EFHD2, RAB27A, or RUNX3, or any combination thereof.

In some instances, the analyzing further comprises detecting the expression or expression level of gene CD28, BTLA, CTLA-4, PD-1, or a gene encoding a HVEM receptor, or any combination thereof. In some instances, the HVEM receptor is encoded by TNFSF14.

In some instances, the analyzing further comprises detecting the expression or expression level of gene NFKBID, NIPAI, FOSL2, NEDD9, BCL2A1, BIRC3, DUSP4/MKP-2, or CFLAR, or any combination thereof.

In some cases, the analyzing further comprises detecting the expression or expression level of a gene selected from the T_{H}2 group of Table 1.

In some cases, the IL-9-expressing antigen-specific T cell (e.g., IL-9-expressing HDM-reactive T cell) is an IL-9-expressing T_{H}2 cell. In some cases, the IL-9-expressing antigen-specific T cell (e.g., IL-9-expressing HDM-reactive T cell) is an IL-9-expressing T_{H} cell.

In some embodiments, the biological sample is processed by a flow cytometry method. In further embodiments, the T cells in the biological sample are enriched by a flow cytometry method, optionally prior to step (a) or step (b) or both. In some instances, the biological sample is contacted with a plurality of beads labeled with CD154 antibodies, optionally biotinylated CD154 antibodies, and processed to generate the population of CD154-enriched T cells. In some instances, the T cells are enriched by contacting the biological sample with a plurality of beads labeled with CD154 antibodies, optionally biotinylated CD154 antibodies, whereby generating the population of CD154-enriched T cells. In some instances, the population of CD154-enriched T cells are enriched by contacting the biological sample with a plurality of beads labeled with CD154 antibodies or biotinylated CD154 antibodies. In some instances, the biological sample is contacted with a plurality of beads labeled with CD137 antibodies, optionally biotinylated CD137 antibodies, and processed to generate the population of CD137-enriched T cells. In some instances, the T cells are enriched by contacting the biological sample with a plurality of beads labeled with CD137 antibodies, optionally biotinylated CD137 antibodies, whereby generating the population of CD137-enriched T cells. In some instances, the population of CD137-enriched T cells are enriched by contacting the biological sample with a plurality of beads labeled with CD137 antibodies or biotinylated CD137 antibodies. In some instances, the biological sample is contacted with a plurality of beads labeled with CD4 antibodies, optionally biotinylated CD4 antibodies, and processed to generate the population of CD4-enriched T cells. In some instances, the T cells are enriched by contacting the biological sample with a plurality of beads labeled with CD4 antibodies, optionally biotinylated CD4 antibodies, whereby generating the population of CD4-enriched T cells. In some instances, the population of CD4-enriched T cells are enriched by contacting the biological sample with a plurality of beads labeled with CD4 antibodies or biotinylated CD4 antibodies. In some cases, the plurality of beads are magnetized beads.

In some instances, the biological sample is a peripheral blood mononuclear cell (PBMC) sample. In some embodiments, the biological sample is selected from a broncheoalveolar lavage fluid sample, a lung draining lymph node sample, an airway biopsy, or a lung biopsy.

In some instances, the sequencing method comprises, or alternatively consists essentially of, or yet further consists of an amplification method. Exemplary amplification methods include, but are not limited to polymerase chain reaction (PCR) such as quantitative PCR (qPCR or real-time PCR), multiplex PCR, direct PCR, nested PCR, touchdown PCR, or hot-start PCR. In some instances, the sequencing method comprises, or alternatively consists essentially of, or yet further consists of generating a plurality of barcoded RNAs. In some cases, the sequencing method comprises, or alternatively consists essentially of, or yet further consists of performing a pairwise differential gene expression analysis, optionally a pairwise single-cell differential gene expression analysis.

In some embodiments, the biological sample is obtained from a subject such as for example, a mammal or a human. In some instances, the subject is suspected of suffering from an inflammation of the skin, airway mucosa, or a combination thereof due to HDM. In some instances, the subject is suspected of suffering from atopic dermatitis, allergic rhinitis, allergic asthma, allergic conjunctivitis, or a combination thereof. In some embodiments, the subject is suspect of comprising a pathogen, such as a virus. In some embodiments, the subject is suspect of suffering from a pathogenic infection (i.e., an infection by a pathogen), optionally a viral infection. In further embodiments, the viral infection is induced by a coronavirus, optionally an alpha-type coronavirus or a beta-type coronavirus, further optionally 229E, NL63, OC43, HKU1, MERS-CoV, SARS-CoV, or SARS-CoV-2. In some embodiments, the viral infection is induced by an influenza virus, optionally an influenza A virus. In some embodiments, the viral infection is induced by cytomegalovirus, Epstein-Barr virus, variola virus, Ebola, dengue, Measles virus, mumps virus, or rubella virus. In some embodiments, the pathogenic infection is induced by a bacterium, a fungus, a protozoan, or a parasite. In some embodiments, the subject lacks or is substantially free of an inflammation of the skin, airway mucosa, or a combination thereof, optionally due to HDM. In some embodiments, the subject is a healthy subject. In further embodiments, the subject develops a disease or a condition as disclosed herein post obtaining the biological sample. Such disease or condition includes, but is not limited to, an allergy, an infection by a pathogen, or an inflammation caused by the pathogenic infection. In some embodiments, the subject is free of an allergy. In further embodiments, the subject develops the allergy post obtaining the biological sample.

In some instances, the biological sample is further stimulated with an antigen, such as an allergen (e.g., HDM, optionally a HDM peptide) (i.e., contacting with the antigen, or contacting with an antigen presenting cell presenting the antigen), optionally prior to processing the biological sample to generate the population of CD154-enriched T cells, the population of CD137-enriched T cells, or the population of CD4-enriched T cells. In some instances, the biological sample is cultured post stimulation for about 2, 3, 4, or 5 days prior to processing the biological sample, such as isolating or enriching or both isolating and enriching T cells or a subset thereof in the biological sample. In some embodiments, the antigen is the one specifically recognized and bound to by the antigen-specific T cells.

In some embodiments, the subject is suspected of or suffering from a pathogenic infection, i.e., an infection by a pathogen. In some instances, the pathogenic infection is a viral infection. In some instances, the viral infection is a respiratory viral infection. In some instances, the viral infection is an airway viral infection. In some instances, the viral infection is a lung viral infection. Additionally or alternatively, the viral infection is in the lung of the subject. In some instances, the viral infection is induced by a coronavirus. In some cases, the coronavirus is an alpha-type coronavirus or a beta-type coronavirus. In some cases, the coronavirus is selected form 229E, NL63, OC43, HKU1, MERS-CoV, SARS-CoV, or SARS-CoV-2. In some cases, the subject is suspected of or suffering from an infection associated with 229E, NL63, OC43, HKU1, MERS-CoV, SARS-CoV, or SARS-CoV-2.

In some instances, the viral infection is induced by an influenza virus. In some cases, the influenza virus is an influenza A virus. In some cases, the subject is suspected of or suffering from an infection associated with an influenza virus, optionally an influenza A virus.

In some instances, the viral infection is induced by cytomegalovirus, Epstein-Barr virus, variola virus, Ebola, dengue, Measles virus, mumps virus, or rubella virus, or any combination thereof. In some cases, the subject is suspected of or suffering from an infection associated with cytomegalovirus, Epstein-Barr virus, variola virus, Ebola, dengue, Measles virus, mumps virus, or rubella virus, or any combination thereof.

In some embodiments, the pathogenic infection is induced by a bacterium, a fungus, a protozoan, or a parasite, or any combination thereof.

In some embodiments, provided is a method of generating a TRAIL-expressing T cell. In some embodiments, the method comprises, or alternatively consists essentially of, or yet further consists of incubating a biological sample comprising a plurality of CD4+ T cells with a TCR stimulator, optionally for at least 10 minutes, to generate a population of stimulated T cells; and isolating or enriched or both isolating and enriching TRAIL-expressing T cells from the stimulated T cells. In some embodiments, the TCR stimulator comprises, or consists essentially of, or yet further consists of an antigen, an anti-CD3 antibody, an anti-CD28 antibody, or any combination thereof. In further embodiments, the antigen can be presented on an antigen presenting cell. In some embodiments, the TCR stimulator contacting step can be substituted by contacting with a TRAIL activator, i.e., a TRIAL agonist or an agent increasing the expression level of TRAIL on a T cell. Non-limiting of suitable agent include a polynucleotide encoding TRAIL or a polynucleotide complementary thereto or a vector comprising the polynucleotide. In some embodiments, the method further comprises contacting the T cells with a TRAIL activator, i.e., an agent increasing the expression level of TRAIL on a T cell.

In some embodiments, the incubation with the biological sample is at least 30 minutes, 1 hour, 2 hours, 3 hours, 4 hours, 5 hours, 6 hours, or longer.

In some embodiments, provided is a method comprising, or consisting essentially of, or further consisting of expanding or isolating or both expanding and isolating a plurality of CD4+ T cells that express an elevated level of an interferon response gene profile. In some embodiments, the method further comprises stimulating the plurality of CD4+ T cells by contacting with a TCR stimulator. In some embodiments, the TCR stimulator comprises, or consists essentially of, or yet further consists of an antigen, an anti-CD3 antibody, an anti-CD28 antibody, or any combination thereof. In further embodiments, the antigen can be presented on an antigen presenting cell.

In some embodiments, the isolated or enriched T cells comprise, or consist essentially of, or further consist of a TRAIL-expressing T_{H} cell or a TRAIL-expressing T_{REG} cell.

In some embodiments, the isolated or enriched T cells comprise, or consist essentially of, or further consist of a T_{H}IFNR cell (also referred to herein as a T_{H}IFNR cell or a TₕIFNR cell or a TₕIFNR) comprising an elevated expression level of at least one of TNFSF10, CXCL10, IFI6, MX1, ISG15, ISG20, OAS1, OAS3, IFIT1, IFIT3, IFI44L, or any combination thereof. In some embodiments, the isolated or enriched T cells comprise, or consist essentially of, or further consist of a T_{H}IFNR cell comprising an elevated expression level of at least one of IFI6, MX1, ISG15, ISG20, OAS1, OAS3, IFIT1, IFIT3, IFITM1, IFI44L, or any combination thereof. In some embodiments, the isolated or enriched T cells comprise, or consist essentially of, or further consist of a T_{H}IFNR cell comprising an elevated expression level of at least one of TNFSF10, CXCL10, or a combination thereof. In some embodiments, the isolated or enriched T cells comprise, or consist essentially of, or further consist of a T_{H}IFNR cell comprising an elevated expression level of TNFSF10. In some embodiments, the isolated or enriched T cells comprise, or consist essentially of, or further consist of a T_{H}IFNR cell comprising an elevated expression level of CXCL10. In some embodiments, the isolated or enriched T cells comprise, or consist essentially of, or further consist of a T_{REG}IFNR cell comprising an elevated expression level of at least one of TNFSF10, ISG15, IFI6, MX1, IFIT3, SAMDL9, OAS1, or a combination thereof. In some embodiments, the isolated or enriched T cells comprise, or consist essentially of, or further consist of a T_{REG}IFNR cell comprising an elevated expression level of TNFSF10. In some embodiments, the isolated or enriched T cells comprise, or consist essentially of, or further consist of a T_{REG}IFNR cell comprising an elevated expression level of at least one of ISG15, IFI6, MX1, IFIT3, SAMDL9, OAS1, or a combination thereof. In some embodiments, the interferon response gene profile further comprises an elevated expression level of at least one of XCL2, SEMATA, CXCR3, FASLG, IFNG, PRF1, KLRG1, XCL1, CD226, NFATC1, or any combination thereof. In some embodiments, the interferon response gene profile comprises, or consists essentially of, or further consists of an elevated expression level of a gene selected from T_{H}IFNR group of Table 1.

Also provided herein are the cells and population of cells isolated using the above-noted methods and having the described characteristics. The populations can be substantially homogenous, e.g., at least 70% or 80% or 85% or 90% or 95% or 99% identical in cell type or expression profile. The cells can be admixed with a carrier, preservative or stabilizer to provide compositions for diagnostic, therapeutic and for use as research reagents. The cells can be of any species, e.g., mammalian or human, as the need may be. Thus, in another aspect provided herein is a composition comprising the cells or population of cells having the described characteristics or features as described herein and one or more of a carrier, preservative or stabilizer.

### Methods of Treatments

In some embodiments, disclosed herein is a method of treating an allergy (e.g., a house dust mite (HDM)-induced allergy) in a subject in need thereof or selecting a subject for treatment of an allergy (e.g., a HDM-induced allergy). In some embodiments, the method comprises, or consists essentially of, or further consists of detecting the presence of an IL-9-expressing antigen-specific T cell (e.g., an IL-9-expressing HDM-reactive T cell) in a biological sample obtained from the subject to identify the subject has expressing the IL-9-expressing antigen-specific T cell (e.g., the IL-9-expressing HDM-reactive T cell); and administering an anti-allergy therapy to the subject expressing the IL-9-expressing antigen-specific T cell (e.g., the IL-9-expressing HDM-reactive T cell).

In some embodiments, the anti-allergy therapy comprises, or consists essentially of, or further consists of administering a therapeutic agent selected from an antihistamine, a corticosteroid, a mast cell stabilizer, and a leukotriene modifier to the subject expressing the IL-9-expressing antigen-specific T cell (e.g., the IL-9-expressing HDM-reactive T cell).

In some embodiments, the IL-9-expressing antigen-specific T cell (e.g., the IL-9-expressing HDM-reactive T cell) is detected by the method comprising, or consisting essentially of, or further consisting of (a) generating a gene expression profile for at least one T cell from one or more (such as each) of a population of CD154-enriched T cells, a population of CD137-enriched T cells, or a population of CD4-enriched T cells obtained from a biological sample by a sequencing method; and (b) analyzing the gene expression profile to detect the presence of the IL-9-expressing antigen-specific T cell (e.g., an IL expressing HDM-reactive T cell) in the biological sample.

In some instances, the antihistamine comprises, or consists essentially of, or further consists of azelastine, cetirizine, chlorpheniramine, diphenhydramine, fexofenadine, levocetirizine, loratadine, olopatadine, promethazine, or triprolidine.

In some instances, the corticosteroid comprises, or consists essentially of, or further consists of bethamethasone, ciclesonide, dexamethasone, fluticasone, methylprednisolone, mometasone, prednisone, prednisolone, or triamcinolone.

In some instances, the mast cell stabilizer comprises, or consists essentially of, or further consists of cromolyn sodium.

In some instances, the leukotriene modifier comprises, or consists essentially of, or further consists of montelukast.

In some instances, the anti-allergy therapy comprises, or consists essentially of, or further consists of administering to the subject a specific immunotherapy (SIT) treatment or regimen.

In some instances, the specific immunotherapy comprises, or consists essentially of, or further consists of sub-cutaneous immunotherapy (SCIT) or sublingual immunotherapy (SLIT).

In some embodiments, the allergy comprises, or consists essentially of, or further consists of an inflammation of the skin, airway mucosa, or a combination thereof. In some cases, the allergy is atopic dermatitis, allergic rhinitis, allergic asthma, allergic conjunctivitis, or a combination thereof. In some embodiments, the allergy is a HDM-induced allergy. In some instances, the HDM-induced allergy comprises, or consists essentially of, or further consists of an inflammation of the skin, airway mucosa, or a combination thereof. In some instances, the HDM-induced allergy is atopic dermatitis, allergic rhinitis, allergic asthma, allergic conjunctivitis, or a combination thereof.

In some embodiments, disclosed herein is a method of treating a virus infection in a subject in need thereof or selecting a subject for treatment of a virus infection. In some embodiments, the virus infection is in the lung. In some embodiments, the method comprises, or alternatively consists essentially of, or yet further consists of detecting the presence of an IL-9-expressing antigen-specific T cell (e.g., an IL-9-expressing virus-reactive T cell) in a biological sample obtained from the subject to identify the subject has expressing the IL-9-expressing antigen-specific T cell; and administering an anti-viral therapy to the subject expressing the IL-9-expressing antigen-specific T cell (e.g., the IL expressing virus-reactive T cell). In some embodiments, the virus infection as referred to herein can be substituted with an infection by a non-virus pathogen.

In some embodiments, further disclosed herein is a method of treating an allergy (e.g., a HDM-induced allergy) in a subject in need thereof comprising, or consisting essentially of, or further consisting of administering to the subject the composition detectable using a method for detecting the presence of an antigen-specific T cell (e.g., a HDM-reactive T cell) characterized with an elevated expression level of an interferon response gene profile as disclosed herein in a biological sample and subsequent harvesting (such as isolating or enriching or both isolating or enriching) of the antigen-specific T cells (e.g., HDM-reactive T cells).

In some embodiments, further disclosed herein is a method of treating an viral infection (e.g., a respiratory viral infection or a SARS-CoV-2 infection) in a subject in need thereof comprising, or alternatively consisting essentially of, or yet further consisting of administering to the subject the composition detectable using a method for detecting the presence of an antigen-specific T cell (e.g., a virus-reactive T cell) characterized with an elevated expression level of an interferon response gene profile in a biological sample and subsequent harvesting of the antigen-specific T cells (e.g., virus-reactive T cells). In some embodiments, the viral infection as referred to herein can be substituted with an infection by a non-virus pathogen.

In some instances, the plurality of antigen-specific T cells (e.g., HDM-reactive T cells) are allogenic to the subject.

In some instances, the plurality of antigen-specific T cells (e.g., HDM-reactive T cells) are autologous to the subject.

In some instances, the subject has an inflammation of the skin, airway mucosa, or a combination thereof, optionally due to HDM.

In some instances, the subject has atopic dermatitis, allergic rhinitis, allergic asthma, allergic conjunctivitis, or a combination thereof, optionally due to HDM.

In some instances, the method further comprises administering to the subject a therapeutic agent, optionally selected from an antihistamine, a corticosteroid, a mast cell stabilizer, and a leukotriene modifier.

In some embodiments, additionally disclosed herein is a method of generating a TRAIL-expressing T cell, comprising, or alternatively consisting essentially of, or yet further consisting of: incubating a biological sample comprising, or alternatively consisting essentially of, or yet further consisting of a plurality of CD4+ T cells with an isolated and recombinant TRAIL protein for at least 10 minutes to generate a population of stimulated T cells; and separating the population of stimulated T cells by a flow cytometry method to isolate or enrich a TRAIL-expressing T cell.

In some instances, the isolated and recombinant TRAIL protein is a full-length protein or a functional fragment thereof.

In some instances, the isolated and recombinant TRAIL protein is a wild-type protein or a variant thereof.

In some instances, the isolated and recombinant TRAIL protein is incubated with the biological sample for at least 30 minutes, 1 hour, 2 hours, 3 hours, 4 hours, 5 hours, 6 hours, or more.

In some instances, the TRAIL-expressing T cell is a TRAIL-expressing T_{H}2 cell or a TRAIL-expressing T_{H} cell or a TRAIL-expressing T_{reg} cell.

In some instances, the method further comprises administering the isolated TRAIL-expressing T cell to a subject in need thereof.

In some instances, the subject has an allergic response to an allergen, optionally to HDM.

In some instances, the subject has an inflammation of the skin, airway mucosa, or a combination thereof, optionally due to HDM.

In some instances, the subject has atopic dermatitis, allergic rhinitis, allergic asthma, allergic conjunctivitis, or a combination thereof, optionally due to HDM.

In some instances, the isolated TRAIL-expressing T cell is allogenic to the subject.

In some instances, the isolated TRAIL-expressing T cell is autologous to the subject.

In certain embodiments, further disclosed herein are compositions comprising an antigen-specific T cell and use of the antigen-specific T cell for treating a disease or indication in a subject in need thereof.

In some embodiments, the antigen-specific T cell is expanded ex vivo prior to administering to the subject for treatment of a disease or condition. In some embodiments, the antigen-specific T cell is a modified antigen-specific T cell, comprising an engineered TCR or an engineered CAR. In some cases, the antigen-specific T cell is an antigen-specific T_{H}2 cell. In some cases, the antigen-specific T cell is an antigen-specific T_{H} cell. In other cases, the antigen-specific T cell is an antigen-specific T_{reg} cell. In additional cases, the antigen-specific T cell is a HDM-reactive T cell, optionally a HDM-reactive T_{H}2 cell or a HDM-reactive T_{reg} cell. In further cases, the antigen-specific T cell is characterized with an expression, an elevated expression, or a reduced expression of at least one gene listed in Table 1.

In some embodiments, the antigen-specific T cell is characterized with an elevated expression level of an interferon response gene profile as described herein. In some instances, the antigen-specific T cell is characterized with an elevated expression level of at least one of TNFSF10, CXCL10, IFI6, MX1, ISG15, ISG20, OAS1, OAS3, IFIT1, IFIT3, IFITM1, IFI44L, SAMDL9, or any combination thereof.

In some cases, the antigen-specific T cell characterized with an elevated expression level of an interferon response gene profile (optionally with at least one of TNFSF10, CXCL10, IFI6, MX1, ISG15, ISG20, OAS1, OAS3, IFIT1, IFIT3, IFI44L, SAMDL9, or a combination thereof) is expanded ex vivo prior to administering to the subject for treatment of a disease or condition.

In some cases, the subject has an allergic response, optionally induced by HDM. In some cases, the subject is suffering from an inflammation of the skin, airway mucosa, or a combination thereof; optionally atopic dermatitis, allergic rhinitis, allergic asthma, allergic conjunctivitis, or a combination thereof. In some cases, the expanded antigen-specific T cell is allogenic to the subject, or autologous to the subject. In some cases, the antigen-specific T cell is an antigen-specific T_{H}2 cell. In some cases, the antigen-specific T cell is an antigen-specific T_{H} cell. In other cases, the antigen-specific T cell is an antigen-specific T_{reg} cell. In additional cases, the antigen-specific T cell is a HDM-reactive T cell, optionally a HDM-reactive T_{H}2 cell (T_{H}IFNR) or a HDM-reactive T_{reg} cell (T_{reg}IFNR).

In some embodiments, the antigen-specific T cell characterized with an elevated expression level of an interferon response gene profile (optionally with at least one of TNFSF10, CXCL10, IFI6, MX1, ISG15, ISG20, OAS1, OAS3, IFIT1, IFIT3, IFITM1, IFI44L, SAMDL9, or a combination thereof) is engineered to express an exogenous TCR or an engineered CAR.

In some instances, the engineered TCR comprises, or consists essentially of, or yet further consists of a modified V domain (e.g., a modified Vα and/or Vβ) that binds to an antigen presented by an antigen presenting cell (e.g., a MHC) or to an allergen (e.g., an aeroallergen such as from dust mite, mold, or pollen; a food allergen such as from milk, egg, soy, wheat, nut, or fish protein; an allergen from a pet or pest; or an allergen from drug) or to an antigen of a pathogen. In some instances, the engineered TCR comprises, or consists essentially of, or yet further consists of a modified V domain (e.g., a modified Vα and/or Vβ) that binds to an antigen (e.g., carcinoembryonic antigen) which upon binding is capable of modulating the immune response to reduce an inflammatory response.

In some cases, the engineered CAR comprises, or consists essentially of, or yet further consists of an antigen-binding domain that binds to an antigen presented by an antigen presenting cell (e.g., a MHC) or to an allergen (e.g., an aeroallergen such as from dust mite, mold, or pollen; a food allergen such as from milk, egg, soy, wheat, nut, or fish protein; an allergen from a pet or pest; or an allergen from drug) or to an antigen of a pathogen. In some cases, the engineered CAR comprises, or consists essentially of, or yet further consists of an antigen-binding domain that binds to an antigen (e.g., carcinoembryonic antigen) which upon binding is capable of modulating the immune response to reduce an inflammatory response.

In some cases, the antigen-specific T cell is an antigen-specific T_{H}2 cell. In some cases, the antigen-specific T cell is an antigen-specific T_{H} cell. In other cases, the antigen-specific T cell is an antigen-specific T_{reg} cell. In additional cases, the antigen-specific T cell is a HDM-reactive T cell, optionally a HDM-reactive T_{H}2 cell (T_{H}IFNR), a HDM-reactive T_{H} cell (T_{H}IFNR) or a HDM-reactive T_{reg} cell (T_{reg}IFNR).

In some embodiments, the antigen-specific T cell is an interleukin 9 (IL-9)-expressing antigen-specific T cell. In some instances, the IL-9-expressing antigen-specific T cell is characterized with an expression of one or more of IL-5, IL-9, IL1RL1, ZEB2, MAF, MAP3K8, GZMB, MT-ND5, DUSP6, SEC61G, DNAJC3, AHI1, CDK2AP2, FKBP11, FKBP1A, KDELR2, CD109, or SEC61B, or any combination thereof. In some instances, the IL-9-expressing antigen-specific T cell is a modified IL-9-expressing antigen-specific T cell, comprising an engineered TCR or an engineered CAR. In some cases, the IL-9-expressing antigen-specific T cell is modified for the treatment of a cancer or an infection.

In some embodiments, the IL-9-expressing antigen-specific T cell is modified for the treatment of a cancer. In some instances, the modified IL-9-expressing antigen-specific T cell compresses an engineered TCR (e.g., comprising a modified Vα and/or Vβ domain) or an engineered CAR that binds to a tumor antigen.

Exemplary tumor antigens include, but are not limited to, alphafetoprotein (AFP), carcinoembryonic antigen (CEA), CA-125, MUC-1, epithelial tumor antigen (ETA), tyrosinase, melanoma-associated antigen (MAGE), and KRAS. In some cases, the cancer is a solid tumor or a hematologic malignancy. In some cases, the solid tumor comprises bladder cancer, breast cancer, colorectal cancer, endometrial cancer, kidney cancer, liver cancer, lung cancer, melanoma, pancreatic cancer, prostate cancer, or thyroid cancer. In some cases, the hematologic malignancy comprises a lymphoma, leukemia, or myeloma. In some cases, the hematologic malignancy comprises a Hodgkin's lymphoma or a non-Hodgkin's lymphoma. In some cases, the hematologic malignancy comprises acute lymphocytic leukemia (ALL), acute myeloid leukemia (AML), chronic lymphocytic leukemia (CLL), chronic myeloid leukemia (CIVIL), or diffuse large B-cell lymphoma (DLBCL).

In some embodiments, the IL-9-expressing antigen-specific T cell is modified for the treatment of an infection. In some instances, the modified IL-9-expressing antigen-specific T cell compresses an engineered TCR (e.g., comprising a modified Vα and/or Vβ domain) or an engineered CAR that binds to an antigen expressed by a pathogen. In some cases, the pathogen is a bacterium, a virus, a protozoan, a fungus, or a worm.

In some embodiments, provided is a method of treating an allergy in a subject in need thereof. In some embodiments, the method comprises, or consists essentially of, or yet further consists of administering to the subject, for example a therapeutically effective amount of, T cells as disclosed herein. In some embodiments, the method comprises, or consists essentially of, or yet further consists of administering to the subject, for example a therapeutically effective amount of, a composition as disclosed herein.

In some embodiments, the subject has an inflammation of the skin, airway mucosa, or a combination thereof, optionally due to HDM. In some embodiments, the subject has atopic dermatitis, allergic rhinitis, allergic asthma, allergic conjunctivitis, or a combination thereof.

In some embodiments, the plurality of T cells are a plurality of HDM-reactive T cells, optionally allogenic or autologous to the subject.

In some embodiments, the method further comprises administering to the subject a therapeutic agent, optionally selected from an antihistamine, a corticosteroid, a mast cell stabilizer, or a leukotriene modifier.

In some embodiments, provided is a method of treating an infection in a subject in need thereof. In some embodiments, the method comprises, or consists essentially of, or yet further consists of administering to the subject, for example a therapeutically effective amount of, T cells as disclosed herein. In some embodiments, the method comprises, or consists essentially of, or yet further consists of administering to the subject, for example a therapeutically effective amount of, a composition as disclosed herein.

In some embodiments, the subject has a pathogenic infection, optionally a viral infection. In some embodiments, the viral infection is induced by a coronavirus, optionally an alpha-type coronavirus or a beta-type coronavirus, further optionally 229E, NL63, OC43, HKU1, MERS-CoV, SARS-CoV, or SARS-CoV-2. In some embodiments, the viral infection is induced by an influenza virus, optionally an influenza A virus. In some embodiments, the viral infection is induced by cytomegalovirus, Epstein-Barr virus, variola virus, Ebola, dengue, Measles virus, mumps virus, or rubella virus. In some embodiments, the pathogenic infection is induced by a bacterium, a fungus, a protozoan, or a parasite.

In some embodiments, the method further comprises administering to the subject a therapeutic agent suitable for treating the infection, optionally an anti-viral therapeutic agent or an antibiotic.

In some embodiments, provided is a method of treating an inflammation in a subject in need thereof. In some embodiments, the method comprises, or consists essentially of, or yet further consists of administering to the subject, for example a therapeutically effective amount of, T cells as disclosed herein. In some embodiments, the method comprises, or consists essentially of, or yet further consists of administering to the subject, for example a therapeutically effective amount of, a composition as disclosed herein.

In some embodiments, the inflammation is in the subject's lung. In some embodiments, the inflammation is induced by an allergy or an infection.

In some embodiments, the method further comprises administering to the subject a therapeutic agent suitable for treating the inflammation, optionally selected from an antihistamine, a corticosteroid, a mast cell stabilizer, or a leukotriene modifier.

In some embodiments, the plurality of the T cells are allogenic to the subject. In some embodiments, the plurality of the T cells are autologous to the subject. In some embodiments, the subject is a human or a mammal.

In some embodiments, the T cells are engineered to express a chimeric antigen receptor (CAR). In some embodiments, the T cells express a T cell receptor (TCR), such as an engineered TCR or a native TCR. In some embodiments, the CAR or the TCR or both specifically recognizes and binds an antigen of a pathogen or a cancer.

In some embodiments, the pathogen is selected from a virus, a bacterium, a fungus, a protozoan, or a parasite. In some embodiments, the virus is a coronavirus, optionally an alpha-type coronavirus or a beta-type coronavirus, further optionally 229E, NL63, OC43, HKU1, MERS-CoV, SARS-CoV, or SARS-CoV-2. In some embodiments, the virus is an influenza virus, optionally an influenza A virus. In some embodiments, the virus is selected from cytomegalovirus, Epstein-Barr virus, variola virus, Ebola, dengue, Measles virus, mumps virus, or rubella virus.

In some embodiments, provided is a method of treating a subject in need thereof. In some embodiments, the method comprises, or consists essentially of, or yet further consists of administering to the subject, for example a therapeutically effective amount of, T cells as disclosed herein. In some embodiments, the method comprises, or consists essentially of, or yet further consists of administering to the subject, for example a therapeutically effective amount of, a composition as disclosed herein.

In some embodiments, the subject is infected or is suspected of being infected by a pathogen, and wherein the CAR or the TCR or both specifically recognizes and binds an antigen of the pathogen. In some embodiments, the method further comprises administering a therapeutic agent suitable for treating the infection.

In some embodiments, the subject has or is suspected of having a cancer, and wherein the CAR or the TCR or both specifically recognizes and binds an antigen of the cancer. In some embodiments, the method further comprises applying an anti-cancer therapy to the subject, optionally selected from an ablation therapy, a radiation therapy, a chemotherapy, an immunotherapy, or a targeted therapy.

In some embodiments, the method does not induce an inflammation in the subject.

In some embodiments, the subject described above is a human.

For the above methods, an effective amount is administered, and administration of the cell or population serves to attenuate any symptom or prevent additional symptoms from arising. When administration is for the purposes of preventing or reducing the likelihood of cancer recurrence or metastasis, the cell or compositions can be administered in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal.

The methods provide one or more of: (1) preventing the symptoms or disease from occurring in a subject that is predisposed or does not yet display symptoms of the disease; (2) inhibiting the disease or arresting its development; or (3) ameliorating or causing regression or relapse of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies e.g., surgical recession, chemotherapy, radiation. In one aspect, treatment excludes prophylaxis.

One of skill in the art can determine if the therapy is successful, e.g., by a reduction of clinical or subclinical symptoms associated with the disease or condition.

### EXAMPLES

These examples are provided for illustrative purposes only and not to limit the scope of the claims provided herein.

### Example 1

Asthma is characterized by aberrant type 2 immune responses to common inhaled aeroallergens such as house dust mite (HDM), grass pollen, animal dander, and mold (Caminati, et al., World Allergy Organ J 11, 13 (2018); J. D. Miller, Clin Rev Allergy Immunol 57, 312-329 (2019); M. Larche, et al., Nat Rev Immunol 6, 761-771 (2006); A. B. Kay, N Engl J Med 344, 109-113 (2001); A. B. Kay, N Engl J Med 344, 30-37 (2001); L. G. Gregory, et al., Trends Immunol 32, 402-411 (2011)), leading to ‘asthma attacks’ in sensitized asthmatic subjects in response to inhalation of such allergens (A. Sykes, et al., J Allergy Clin Immunol 122, 685-688 (2008)). The hallmarks of asthma, namely airway narrowing and sputum eosinophilia, have been shown to result from the specific activation of (MHC) class II-restricted CD4⁺ helper T cells (T_{H}) by challenging asthmatics with synthetic allergen-derived peptides (S. Oddera, et al., Lung 176, 237-247 (1998); S. H. Arshad, et al., Am J Respir Crit Care Med 157, 1900-1906 (1998); P. Maestrelli, et al., Clin Exp Allergy 31, 715-721 (2001); F. R. Ali, et al., Am J Respir Crit Care Med 169, 20-26 (2004); L. M. Muehling, et al., J Allergy Clin Immunol 140, 1523-1540 (2017).). Further evidence of the centrality of T_{H} cells in asthma pathology is that their depletion reduces allergic airway inflammation in animal models (K. Raemdonck, et al., Respir Res 17, 45 (2016)), and that inhibition of T_{H} cell-derived type 2 cytokines (IL-5, IL-13, IL-4) is clinically beneficial in patients with asthma (D. Gibeon, et al., Expert Rev Respir Med 6, 423-439 (2012); A. S. Kim, et al., Ann Allergy Asthma Immunol 116, 14-17 (2016); S. Maltby, et al., Chest 151, 78-89 (2017); N. Drick, et al., BMC Pulm Med 18, 119 (2018)). However, despite the central role of allergen-reactive T_{H} cells and their products in driving airway inflammation, the full spectrum and function of T_{H} cell subsets that respond to common allergens has yet to be defined. Similarly, though an imbalance between regulatory T cells (T_{reg}) and T_{H} cell responses to allergens is associated with the development of allergy and asthma (V. Schulten, et al., J Allergy Clin Immunol 141, 775-777 e776 (2018); M. Noval Rivas, et al., J Allergy Clin Immunol 138, 639-652 (2016); C. A. Tibbitt, et al., Immunity 51, 169-184 e165 (2019); I. P. Lewkowich, et al., J Exp Med 202, 1549-1561 (2005); M. D. Leech, et al., J Immunol 179, 7050-7058 (2007); D. H. Strickland, et al., J Exp Med 203, 2649-2660 (2006)), the heterogeneity of allergen-reactive T_{reg} cells remains unstudied.

Previous studies of allergen-reactive T cells have characterized their phenotype based on the expression of cell-surface markers or canonical cytokines (E. Wambre, et al., Sci Transl Med 9, (2017); K. A. Smith, et al., BMC Immunol 14, 49 (2013); B. Upadhyaya, et al., J Immunol 187, 3111-3120 (2011)). Due to their relative rarity, analyses of these cells usually require in vitro expansion, which can alter their molecular properties, thus limiting the value of unbiased transcriptomic studies (M. Januszyk, et al., Microarrays (Basel) 4, 540-550 (2015); C. E. Nestor, et al., Genome Biol 16, 11 (2015); D. J. Mazzatti, A. et al., Aging Cell 6, 155-163 (2007)). Furthermore, transcriptomic studies performed at the whole population level fail to capture cellular heterogeneity and also lack the resolution to detect biological differences associated with asthma or allergy (G. Seumois, et al., J Immunol 197, 655-664 (2016)). A recent single-cell analysis of T_{H} cells in a mouse models of allergic airway inflammation revealed substantial heterogeneity, and also identified T_{H} subsets that had not been previously described (C. A. Tibbitt, et al., Immunity 51, 169-184 e165 (2019)).

Characterizing the various subsets of T_{H} and T_{reg} cells in asthmatic subjects and comparing their frequency and properties to those in subjects without asthma is ideally achieved at single-cell resolution. Indeed, single-cell transcriptomic analysis can help define the molecular properties of allergen-reactive T_{H} cells associated with pathology and assess whether these features are the result of an expansion of a pre-existing population of cells or the result of their aberrant differentiation in response to environmental signals (J. Geginat, et al., Front Immunol 5, 630 (2014); M. DuPage, et al., Nat Rev Immunol 16, 149-163 (2016)). To address the latter issue, the subsets of allergen-reactive T_{H} cells must also be defined in subjects without asthma and allergy. Such allergen-reactive T_{H} cells are present even in non-allergic subjects (D. Hinz, et al., Clin Exp Allergy 46, 705-719 (2016); G. Birrueta, et al., PLoS One 13, e0204620 (2018); V. Schulten, et al., Front Immunol 9, 235 (2018); M. Akdis, et al., J Exp Med 199, 1567-1575 (2004)), although it is not known why or how these cells fail to cause overt allergic responses.

To address these questions in a hypothesis-free manner, a single-cell transcriptomic analysis of T_{H} and T_{reg} cells that react to house dust mite allergen (HDM) was performed. HDM is one of the most common and ubiquitous allergens, and sensitization is associated with both the onset of allergic asthma and its severity (V. D. Gandhi, et al., Curr Allergy Asthma Rep 13, 262-270 (2013); M. A. Calderon, et al., J Allergy Clin Immunol Pract 3, 843-855 (2015); M. Dullaers, et al., J Allergy Clin Immunol 140, 76-88 e77 (2017); P. M. Salo, et al., J Allergy Clin Immunol 121, 678-684 e672 (2008)). The relatively high abundance of HDM-reactive T cells in the blood makes it is possible to isolate sufficient number of cells for high-throughput single-cell transcriptomic analysis. Herein the inventors quantify the single-cell transcriptomes of >50,000 HDM-reactive T cells from allergic asthmatic subjects and relevant control groups. The analysis revealed multiple distinct subsets of T_{H} and T_{reg} cells that are either preferentially expanded or depleted in asthmatic subjects with and without HDM allergy, defined the pathogenic properties of T_{H} subsets associated with allergic asthma, allergic conjunctivitis, and uncovered a unique HDM-reactive T_{H} subset that is expanded specifically in subjects without HDM allergy.

Bulk RNA-Seq Analysis of HDM Allergen-Reactive T Cells does not Identify Asthma-Specific Features

To comprehensively characterize the molecular properties of allergen-reactive T_{H} and T_{reg} cells from patients with asthma, pure populations of HDM-reactive memory T_{H} and T_{reg} cells were isolated ex vivo (see Materials and Methods) from asthmatics with HDM allergy (N=6) and performed both bulk and single-cell RNA-seq. To distinguish the molecular features that are specific to asthma as opposed to HDM allergy, similar assays were performed in HDM-reactive T_{H} and T_{REG} cells isolated from HDM-allergic subjects without asthma (N=6). Because allergen-reactive T cells are present even in non-allergic subjects (D. Hinz, et al., Clin Exp Allergy 46, 705-719 (2016); G. Birrueta, et al., PLoS One 13, e0204620 (2018); V. Schulten, et al., Front Immunol 9, 235 (2018); M. Akdis, et al., JExp Med 199, 1567-1575 (2004)), HDM-reactive T_{H} and T_{reg} cells were also isolated from asthmatic (N=6) and healthy subjects (N=6) without HDM allergy to uncover features that may contribute to the lack of HDM allergy i.e., IgE reactivity. In total, 95 bulk RNA-seq and ˜50,000 single-cell RNA-seq assays were performed on T cells from a total of 24 subjects (data not shown).

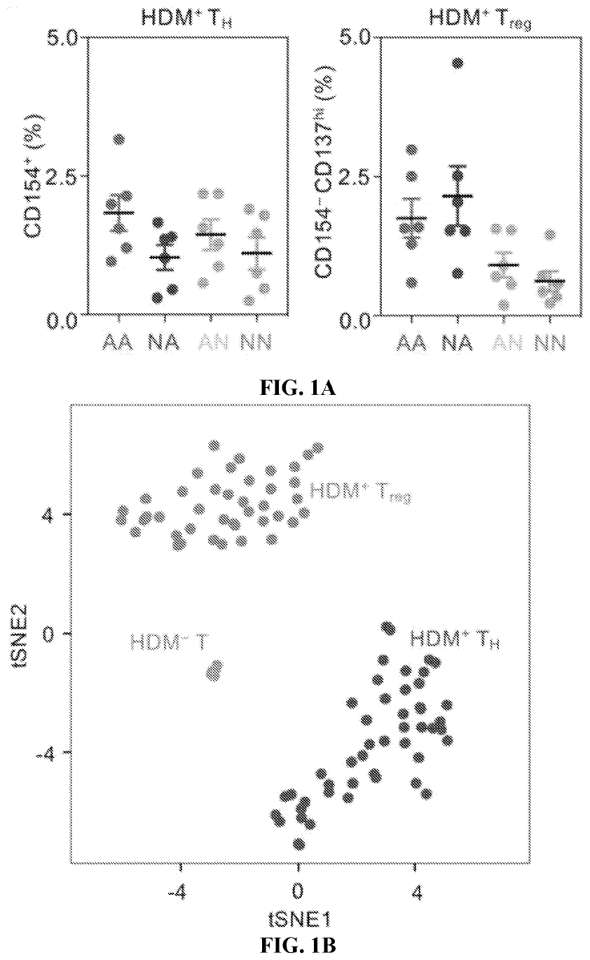

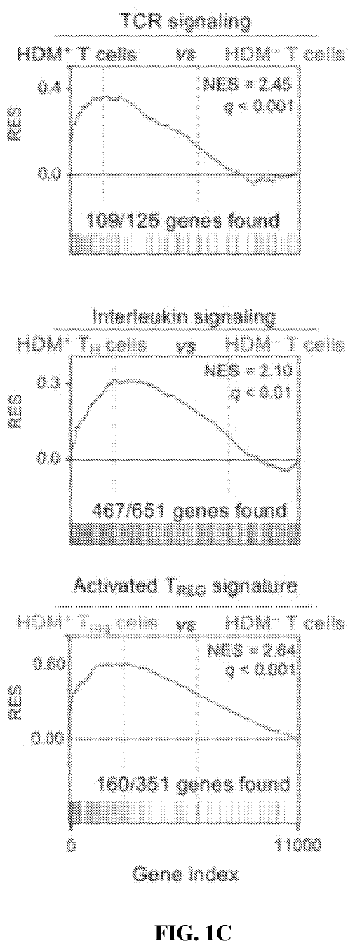

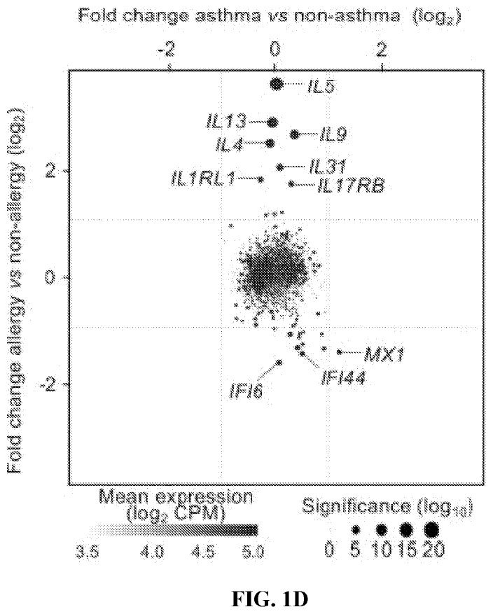

HDM-reactive T_{H} cells (0.2-3% of all memory T_{H} cells) and T_{reg} cells (1-5% of all memory T_{reg} cells) were detected in all 4 subject groups, including the HDM-allergic and non-allergic subjects (FIG. 1A). Bulk transcriptome analysis showed that HDM-reactive T_{H} and T_{reg} cells clustered separately from one another and from HDM-non-reactive cells (HDM⁻ T cells) (FIG. 1B). 724 transcripts were differentially expressed between both HDM-reactive, activated T cells populations, T_{H} and T_{reg} (following stimulation with HDM peptide/MHC complex from antigen-presenting cells) and HDM⁻ T cells (not stimulated by HDM-allergen derived peptides) (adjusted P-value <0.01, log₂ fold change >2, data not shown). As expected, these differentially expressed transcripts were highly enriched for genes in the TCR signaling pathway (FIG. 1C, top panel). Allergen-activated HDM-reactive T_{H} cells expressed greater amounts of several transcripts encoding cytokines (IL-2, -13, -5, -4, -9, -31 -17F, -22, TNF, IFNG, CSF-2) and chemokines (CCL20, CXCL10) linked to effector functions (FIG. 1C, middle panel). HDM-reactive T_{reg} cells expressed higher levels of genes linked to T_{reg} function, such as IL2RA, FOXP3, CTLA4, IKZF2, TNFRSF8, when compared with HDM⁻ T cells (FIG. 1C and data not shown).

Clustering analysis of HDM-reactive T_{H} cells by disease group showed separation based on HDM allergy status rather than asthma phenotype (data not shown). For example, in HDM-reactive T_{H} cells from HDM-allergic subjects, expression of canonical T_{H}2 cytokines was increased compared with those from HDM-non-allergic subjects (FIG. 1D), whereas no significant differences were observed between the HDM-reactive T_{H} cells from asthmatic versus non-asthmatic subjects with HDM allergy (FIG. 1D). The heterogeneity observed within the HDM-reactive T_{H} population, reflected in the co-expression of transcripts encoding canonical T_{H}1, T_{H}2 and T_{H}17 cytokines (data not shown), is likely to have limited the resolution of bulk transcriptome data to distinguish asthma-specific features.

Single-Cell RNA-Seq Analysis Reveals Heterogeneity Among HDM-Reactive T_{H} Cells

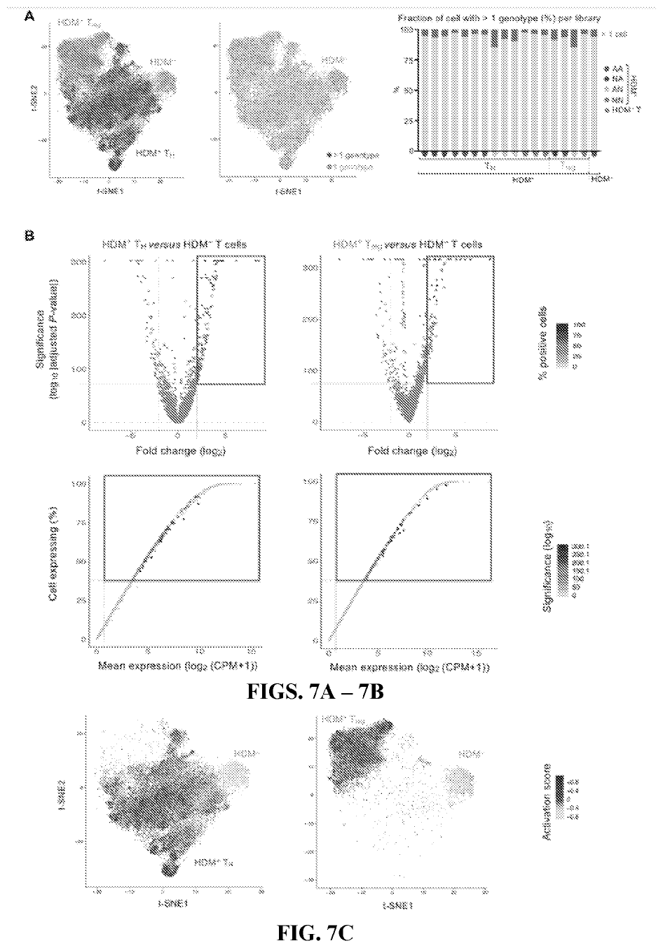

Single cells from all 6 subjects in each disease group were pooled for droplet-based single-cell RNA-seq (10× Genomics platform), and genotype-based deconvolution was employed to obtain subject-specific single-cell transcriptomes and to exclude potential cell doublets (see Materials and Methods, and FIG. 7). The cell isolation strategy, based on the CD154 activation marker, primarily enriches for HDM-reactive T_{H} cells (M. Frentsch, et al., Nat Med 11, 1118-1124 (2005); P. K. Chattopadhyay, et al., Nat Med 11, 1113-1117 (2005); P. Bacher, et al., Cell 167, 1067-1078 e1016 (2016)). Analogous to flow cytometry-based approaches, single-cell transcriptome analysis allows discrimination of activated (true positives) from non-activated (false positives) T_{H} cells. Based on a T_{H} activation signature, derived by comparing HDM-reactive T_{H} and HDM-non-reactive (HDM⁻ T cells) single cells (data not shown and FIG. 7), potential false positive cells were eliminated from the HDM-reactive T_{H} cell population (data not shown and FIG. 7).

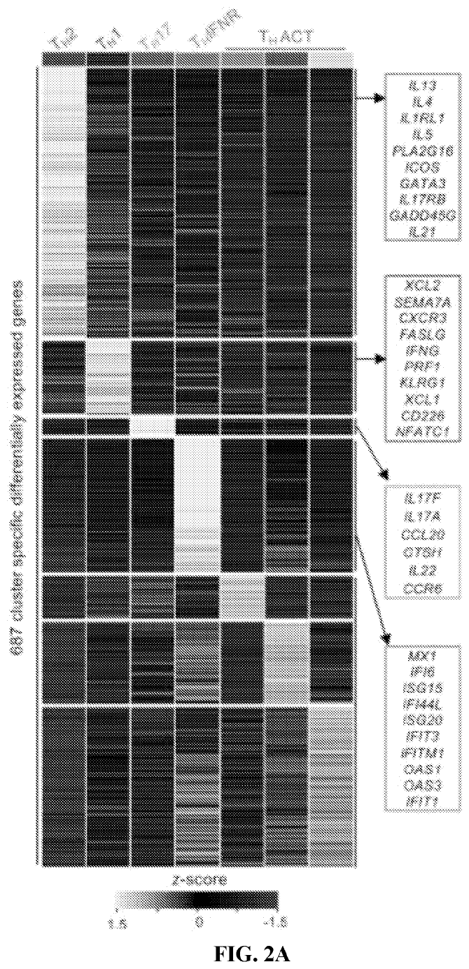

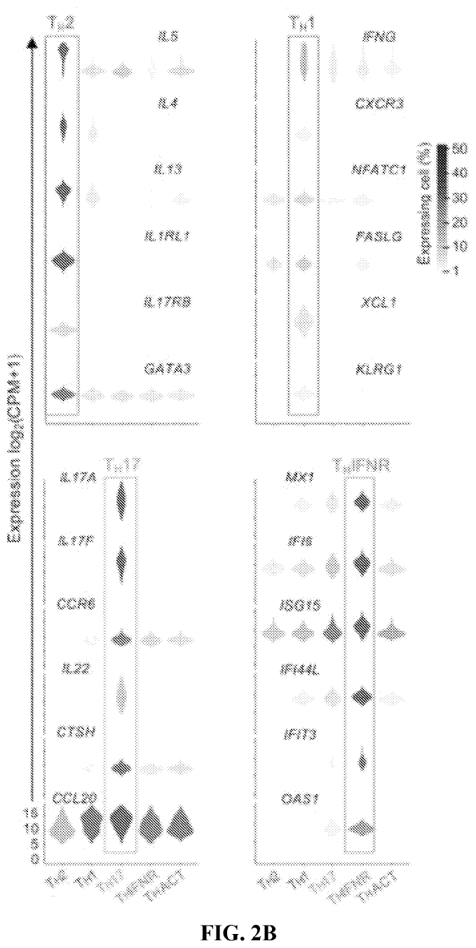

Analysis of the single-cell transcriptomes of HDM-reactive T_{H} cells (non-doublet and activation-signature positive) revealed 7 clusters (Material and Methods) present at varying frequency among subjects, highlighting the importance of studying cells from multiple subjects (data not shown and FIG. 8). To understand the molecular properties unique to each cluster, multiple pair-wise single-cell differential gene expression analyses were performed (Materials and Methods, and Table 1). Several hundred genes (N=687) were especially highly expressed by each cluster, allowing classification into specific T_{H} subsets (FIG. 2A). Cells in cluster 1 were highly enriched for transcripts encoding canonical type 2 cytokine genes (IL5, IL13, IL4), the T_{H}2 master transcription factor GATA3, and receptors (IL1RL1 and IL17RB) for the T_{H}2-polarizing cytokines IL-33 and IL-25, indicating that this cluster represented T_{H}2 cells (FIG. 2B). Notably, the T_{H}2 subset only represented ˜6.3% of the HDM-reactive T_{H} cell population (data not shown). Cluster 2 was enriched for T_{H}1 phenotype- and function-related genes such as IFNG, CXCR3, and PRF1 (C. L. Arlehamn, et al., J Immunol 193, 2931-2940 (2014); Y. Serroukh, et al., Elife 7, (2018); A. L. Kroczek, et al., Front Immunol 9, 2806 (2018); B. G. Dorner, et al., Proc Natl Acad Sci USA 99, 6181-6186 (2002)) (FIG. 2A and FIG. 2B). In addition, the inventors found that the expression of genes encoding the chemokines XCL1 and XCL2 was correlating with expression of IFNG (FIG. 9), suggesting a potentially important role of these chemokines in the function of T_{H}1 cells (A. L. Kroczek, et al., Front Immunol 9, 2806 (2018); B. G. Dorner, et al., Proc Natl Acad Sci USA 99, 6181-6186 (2002); T. Yoshida, et al., J Biol Chem 273, 16551-16554 (1998); A. S. Haider, et al., J Investig Dermatol Symp Proc 12, 9-15 (2007)). Cluster 3 was enriched for T_{H}17 phenotype- and function-related genes such as IL17A, IL17F, CCR6, IL22, CTSH, and CCL20 (S. C. Liang, et al., J Exp Med 203, 2271-2279 (2006); Y. Lee, et al., Nat Immunol 13, 991-999 (2012); R. Ramesh, et al., J Exp Med 211, 89-104 (2014)). The characteristics of cell clusters 1-3 were confirmed by gene set enrichment analysis (GSEA) using curated lists of signature genes (data not shown). Cells in cluster 4 were very highly enriched for type I and II interferon response genes (IFI6, MX1, ISG20, OAS1, IFIT1, IFI44L) (S. Y. Liu, et al., Proc Natl Acad Sci USA 109, 4239-4244 (2012); A. J. Lee, et al., Front Immunol 9, 2061 (2018)), indicating that they represent a previously uncharacterized T_{H} subset, which was termed T_{H} subset expressing the interferon response signature (T_{H}IFNR) (FIG. 2A and FIG. 2B). GSEA analysis confirmed enrichment of interferon response genes in this cluster (data not shown). Cells in clusters 5, 6, and 7 were enriched for genes linked with cell activation; cluster 5 (T_{H}ACT1) was enriched in genes linked to ribosomal proteins and RNA translation (RPLx, RPSx, see Table 1), cluster 6 (T_{H}ACT2) was enriched with genes linked to endocytosis and membrane trafficking (ARL6IP5, ARPC5, BIN1, see Table 1), and cluster 7 (T_{H}ACT3) was enriched in genes linked to chromosome maintenance (NPM1, NHP2) and apoptosis (GADD45B, NFKB1, ATF4, PMAIP1, see Table 1). Overall, the single-cell transcriptome analysis uncovered substantial heterogeneity among HDM-reactive T_{H} cells.

Proportions of HDM-Reactive T_{H} Subsets Differ Between HDM Allergic and Non-Allergic Subjects

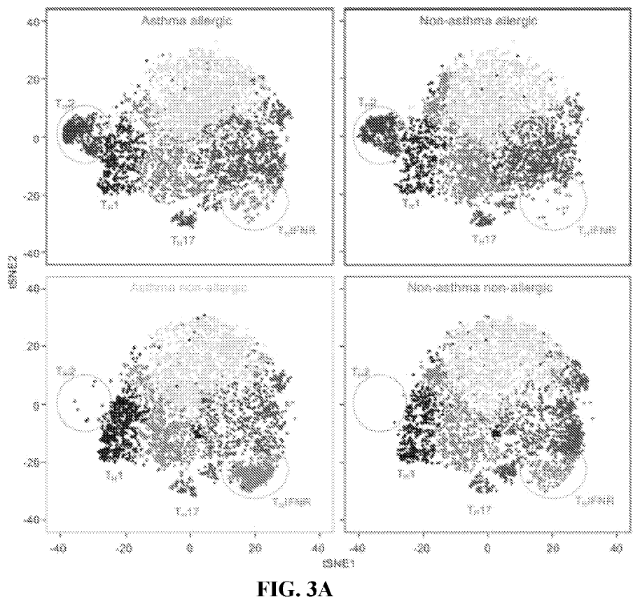

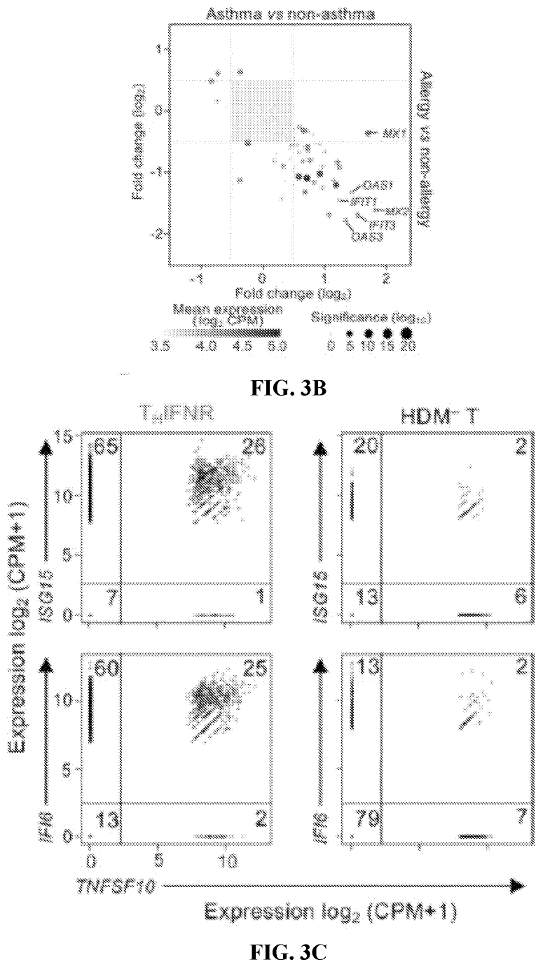

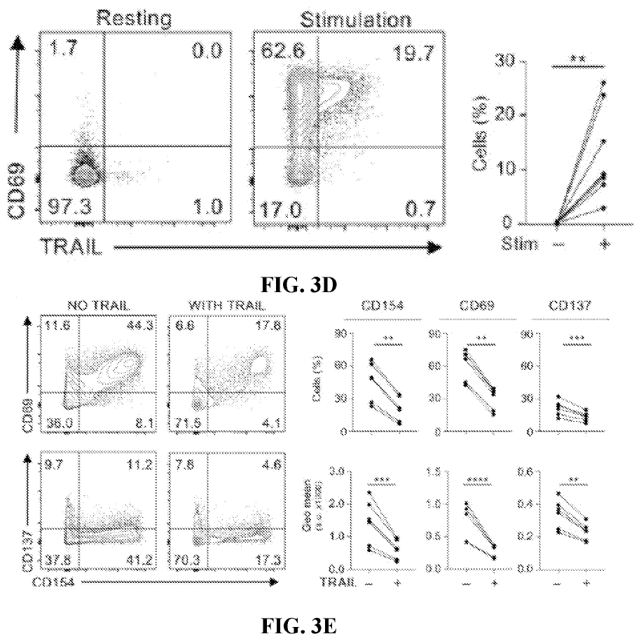

It was next asked if the proportions of any of the HDM-reactive subsets varied between subjects with or without HDM allergy or asthma. As expected, the T_{H}2 cluster (cluster 1) was present only in the HDM-allergic groups (FIG. 3A), consistent with the central role of T_{H}2 cells and type 2 cytokines in IgE class switching and allergy and asthma pathogenesis (S. N. Georas, et al., Eur Respir J 26, 1119-1137 (2005); D. B. Corry, et al., Nature 402, B18-23 (1999); H. C. Oettgen, et al., J Clin Invest 104, 829-835 (1999)). On the other hand, the T_{H}1 cluster, though observed in all subject groups, was present at greater proportions in subjects without HDM allergy, consistent with the reciprocal role of T_{H}1 cells in dampening T_{H}2 differentiation. Several other clusters, including the T_{H}17 cluster, showed no significant differences in their proportions among disease and control groups (FIG. 3A).

Subjects without HDM allergy—both the asthmatic and healthy control groups—despite displaying a substantially broad T_{H} response to HDM allergen, failed to generate T_{H}2 cells that respond to HDM ex vivo. Strikingly, the large majority of HDM-reactive T_{H} cells expressing the interferon response signature (T_{H}IFNR, cluster 4) were observed in subjects without HDM allergy (FIG. 3A, and FIG. 3B). This negative association raised the possibility that the T_{H}IFNR subset plays a role in dampening T_{H}2 responses to allergens. Intriguingly, cells in the T_{H}IFNR subset expressed the highest levels of CXCL10 and TNFSF10 (data not shown). CXCL10 encodes CXCL10, a chemokine that recruits T_{H} cells expressing the chemokine receptor CXCR3, which mainly comprises T_{H}1 cells (J. Li, et al., Cytokine 94, 45-51 (2017); M. Gauthier, et al., JCI Insight 2, (2017)). Thus, CXCL10 expression by T_{H}IFNR cells is likely to promote selective recruitment of T_{H}1 cells (J. R. Groom, et al., Immunity 37, 1091-1103 (2012)). TNFSF10 encodes tumor necrosis factor-related apoptosis-inducing ligand (TRAIL), which can drive apoptosis in cells expressing its receptor (TRAIL-R) (K. Beyer, et al., Cancers (Basel) 11 (2019); C. Peteranderl, et al., Front Immunol 8, 313 (2017)). More recently, both surface-bound and soluble TRAIL have been shown to dampen TCR signaling by inhibiting the phosphorylation of downstream kinases (C. Lehnert, et al., J Immunol 193, 4021-4031 (2014); I. T. Chyuan, et al., Front Immunol 9, 15 (2018); A. I. Roberts, et al., Immunol Res 28, 285-293 (2003); X. R. Zhang, et al., Cell Death Differ 10, 203-210 (2003)). Given that activated T_{H} cells express TRAIL-R (A. I. Roberts, et al., Immunol Res 28, 285-293 (2003); X. R. Zhang, et al., Cell Death Differ 10, 203-210 (2003)), TRAIL produced by the HDM-reactive T_{H}IFNR cells may play an important role in blocking T_{H} cell responses to HDM in vivo. Interestingly, a small fraction of cells expressing T_{H}IFNR signature genes (IFI6, ISG15) and TNFSF10 were observed even in resting cell not reactive to HDM, suggesting persistence of this population within PBMC (FIG. 3C). The inventors confirmed that following TCR stimulation TRAIL was expressed by population of T_{H} cells, which is likely to be enriched for the T_{H}IFNR subset (FIG. 3D). In published single-cell datasets (X. Guo, et al., Nat Med 24, 978-985 (2018)), it was found that CD4⁺ T cells in human lungs expressed interferon-response signature genes and TRAIL, which indicated that T_{H}IFNR subset is also present in the human lung tissue (data not shown). The inventors next experimentally tested TRAIL's function and found that recombinant TRAIL inhibited TCR-dependent activation of T_{H} cells ex vivo, as measured by the surface expression of the activation markers CD154, CD69, and CD137 (4-1BB) (P. Bacher, et al., J Immunol 190, 3967-3976 (2013)) (FIG. 3E). These findings support a potential regulatory role of HDM-reactive T_{H}IFNR cells in dampening allergic responses.

A Subset of HDM-Reactive T_{reg} Cells Express the Interferon Response Signature

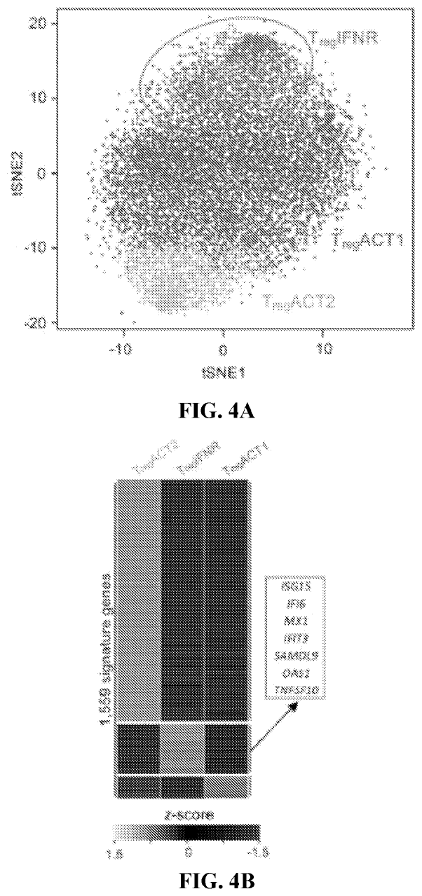

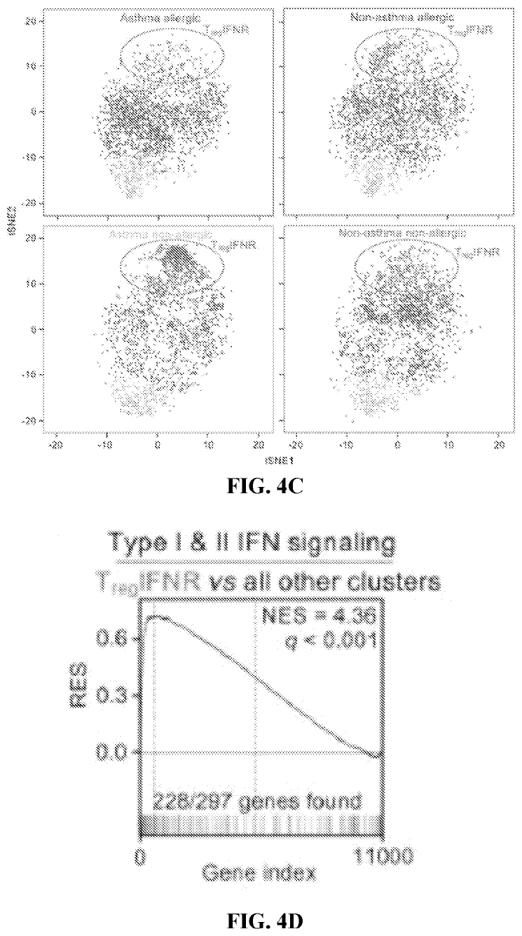

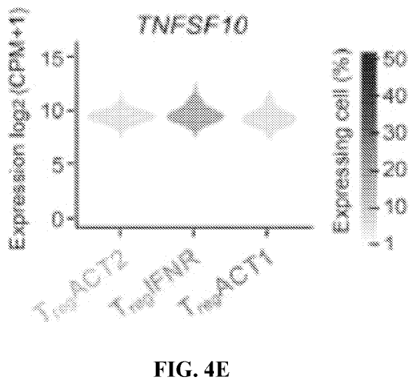

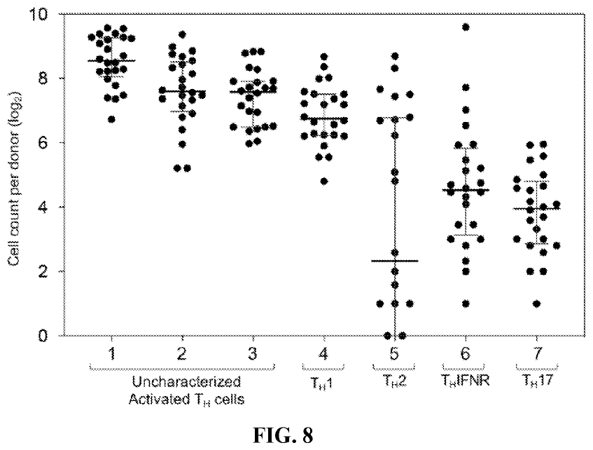

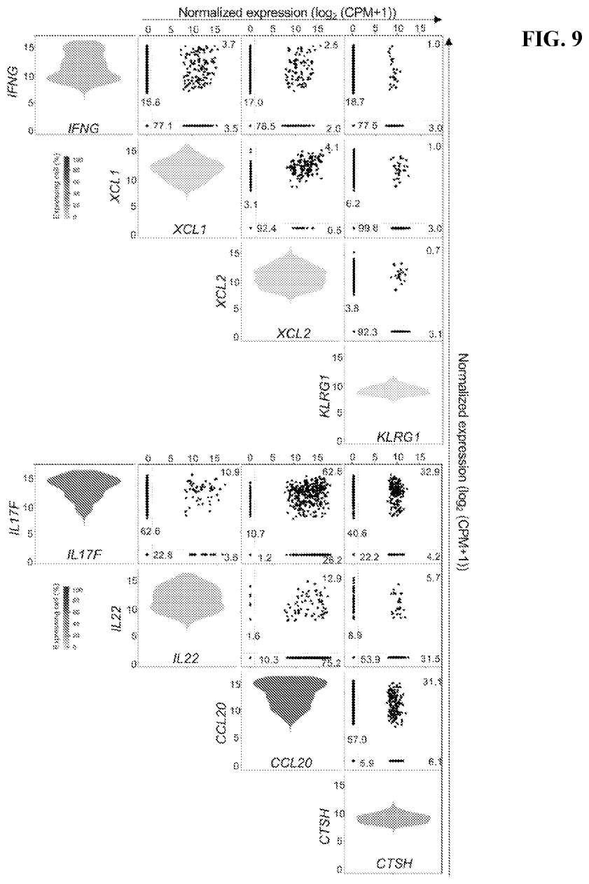

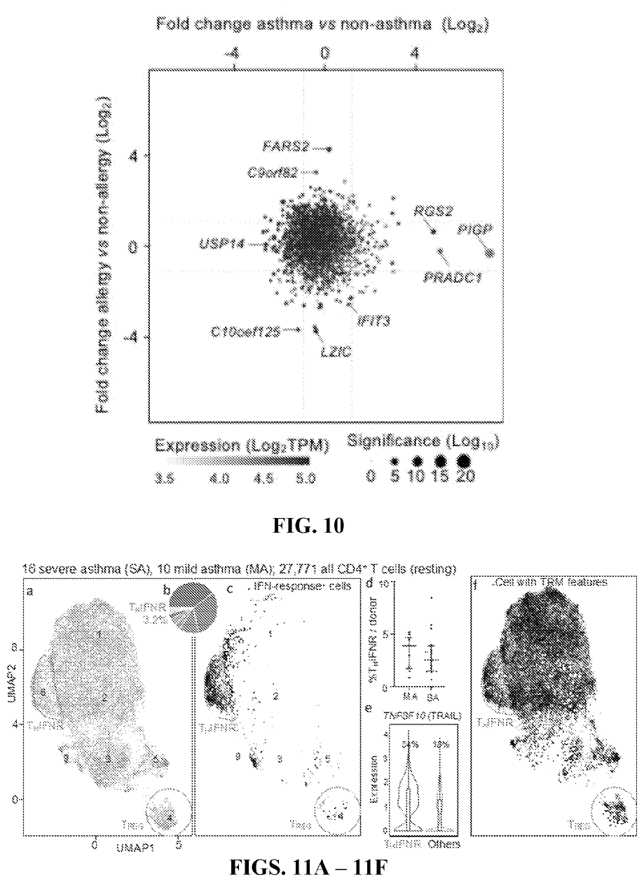

It was also investigated whether HDM-reactive T_{reg} cells differed between HDM allergic and non-allergic subjects. As shown previously, it was confirmed that the proportion of HDM-reactive T_{reg} cells was not related to HDM allergic status (FIG. 1A) (P. Bacher, et al., Cell 167, 1067-1078 e1016 (2016)). Furthermore, transcriptomic analysis of bulk populations of HDM-reactive T_{reg} cells revealed no major disease-related differences (FIG. 10). To determine whether specific subsets of HDM-reactive T_{reg} cells varied with disease state, single-cell transcriptomic analysis of ˜10,000 HDM-reactive T_{reg} cells across the 4 subject groups was performed, which separated this population into 3 distinct clusters (FIG. 4A and FIG. 4B, and Table 1). The proportion of cells in cluster 2 was greater in asthmatic subjects without HDM allergy compared with HDM-allergic asthmatics, suggesting preferential expansion of this subset in asthmatic subjects without HDM allergy (FIG. 4C). GSEA analysis of transcripts enriched in this cluster (N=248) revealed significant enrichment of interferon response genes (FIG. 4D). The features of this T_{reg} cluster were similar to those of the T_{H}IFNR cluster, which was also present at higher proportions in subjects without HDM allergy; for example, interferon-responsive T_{reg} cells (T_{reg} IFNR) also expressed higher levels of transcripts encoding for TRAIL (FIG. 4E). Overall, these findings indicate preferential expansion of HDM-reactive T_{reg} and T_{H} cells expressing the interferon response signature in asthmatic subjects without HDM allergy, and that expression of TRAIL by these subsets is likely to play an important role in dampening T_{H}2 responses to HDM allergens. Further studies in animal models are performed to confirm this hypothesis.

HDM-Reactive T_{H}2 Cells are Enriched for Transcripts Linked to Enhanced Functionality

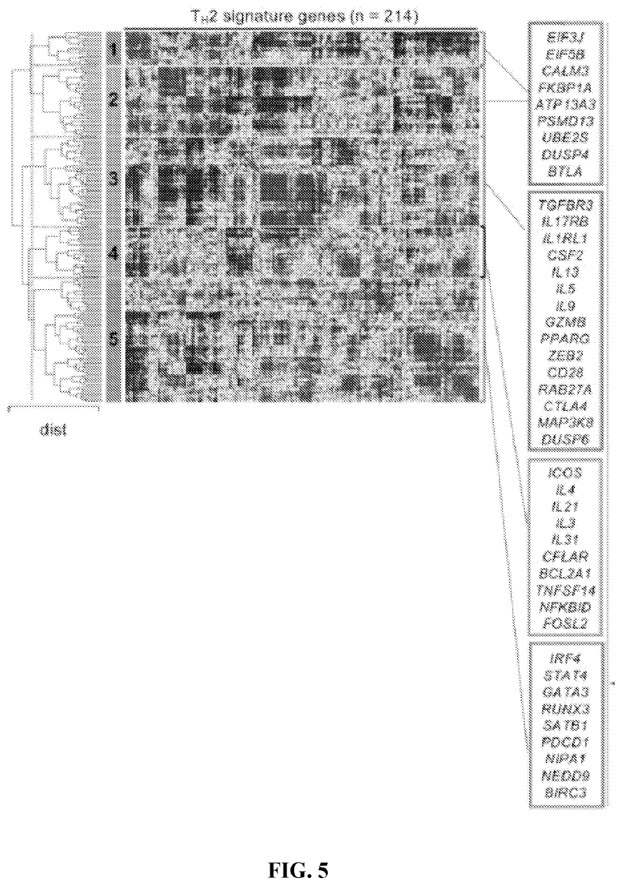

Given the important role of T_{H}2 cells in the pathogenesis of allergy and asthma, the genes enriched in the T_{H}2 cluster were analyzed to gain insights into their functional properties. Gene co-expression analysis is a powerful method to discover new genes that are likely to play important role in the differentiation or function of a given cell type (S. van Dam, et al., Brief Bioinform 19, 575-592 (2018); J. Clarke, et al., J Exp Med 216, 2128-2149 (2019)). Hierarchical gene clustering (FIG. 3A) and weighted gene co-expression network analysis (WGCNA) (P. Langfelder, et al., BMC Bioinformatics 9, 559 (2008)) (Materials and Methods) of the 214 ‘T_{H}2 enriched’ transcripts, defined from single-cell transcriptome analysis (data not shown), revealed 5 modules of highly co-expressed genes (FIG. 5). Among these 5 modules, 2 modules (Module 1 and Module 2) contained genes linked to cellular metabolism, protein trafficking, active transcription and oxidative phosphorylation (EIF3J, E1F5B, CALM3, FKBP1A, PTPN11, ATP13A3, PSMD13, UBE2S, DUSP4), indicating increased metabolic and transcriptional activity in T_{H}2 cells; a third (Module 5) module contained genes encoding for important transcription factors linked to T_{H}2 cell differentiation such as GATA3, IRF4, and SATB1 (M. Huber, et al., Eur J Immunol 44, 1886-1895 (2014); H. Ahlfors, et al., Blood 116, 1443-1453 (2010); A. O'Garra, et al., J Immunol 196, 4423-4425 (2016)).

The module including the canonical type 2 cytokine genes (IL5, IL13) (Module 3 in FIG. 5), likely includes other genes that play an important role in driving the effector functions of T_{H}2 cells. One of the most highly co-expressed transcripts encodes for the effector cytokine IL-9, which has recently been shown to be produced by a subset of T_{H}2 cells that expressed PPAR-γ following TGF-β signaling (C. Micosse, et al., Sci Immunol 4 (2019)). The inventors found that transcripts encoding for PPAR-γ and the TGFβ receptor 3 (TGFBR3) were also enriched in T_{H}2 cells and highly co-expressed with IL9 (FIG. 5), suggesting that the IL-9 differentiation pathway is active in T_{H}2 cells. Finally, transcripts linked to cytotoxicity function (GZMB, RAB27A (M. D. Prakash, et al., Immunity 41, 960-972 (2014); V. S. Patil, et al., Sci Immunol 3, (2018); J. C. Stinchcombe, et al., J Cell Biol 152, 825-834 (2001)))) and differentiation of cytotoxic T_{H} cells (ZEB2, RUNX3 (A. L. Kroczek, et al., Front Immunol 9, 2806 (2018); V. S. Patil, et al., Sci Immunol 3, (2018); K. D. Omilusik, et al., J Exp Med 212, 2027-2039 (2015))) were also highly co-expressed with IL5 and IL13, indicating that HDM-reactive cells may include cytotoxic T_{H}2 cells (FIG. 5). Cytotoxic T_{H} cells are known to contribute to antiviral immunity (V. S. Patil, et al., Sci Immunol 3, (2018); N. B. Marshall, et al., J Biomed Biotechnol 2011, 954602 (2011)) and autoimmunity (M. Thewissen, et al., J Immunol 179, 6514-6523 (2007)), and these findings suggest that they may also play a role in asthma pathogenesis.

The gene for another canonical type 2 cytokine, IL-4, also linked to the function of follicular T cells (T_{fh}) and IgE class switching, was present in a fourth module (Module 4). Important molecules encoded by genes in this module include IL-31, a member of the IL-6 family of cytokines that is produced by activated T_{H}2 cells and leads to itching in skin inflammation (FIG. 5) (A. Takamori, et al., Sci Rep 8, 6639 (2018); B. F. Gibbs, et al., Front Immunol 10, 1383 (2019)), and IL-3, which is linked to hematopoietic progenitor proliferation and recruitment (J. T. Schroeder, et al., J Immunol 182, 2432-2438 (2009); G. T. Williams, et al., Nature 343, 76-79 (1990); G. F. Weber, et al., Science 347, 1260-1265 (2015)). It was recently shown that IL-3 plays an important role in the activation and survival of eosinophils (R. K. Nelson, et al., J Immunol 203, 329-337 (2019)). Other genes in this module encode for products such as ICOS and IL-21, which is linked to B cell help and immunoglobulin isotype class switching (FIG. 5), suggesting that this module was enriched for genes linked to T_{fh} cell function. The presence of gene modules with distinct co-expression patterns indicated potential heterogeneity in the T_{H}2 population. To address this issue, the inventors re-clustered cells only from the T_{H}2 population; this analysis revealed 2 distinct sub-clusters (data not shown), each highly enriched for genes in Modules 3 and 4 (data not shown).

Overall, these results show that cells in the T_{H}2 cluster were enriched for the expression of transcripts encoding for several co-stimulatory and inhibitory receptors as well as transcription factors and molecules known to promote T cell survival. The expression of the first class of molecules, including CD28, ICOS, BTLA, CTLA-4, PD-1, HVEM receptor (LIGHT, TNFSF14) (L. Chen, et al., Nat Rev Immunol 13, 227-242 (2013)), suggests that these molecules could be targeted to dampen the pro-inflammatory function of T_{H}2 cells in asthma. Pro-survival factors included several in the NFκB signaling pathway, including NFKBID, NIPAI, MAP3K8, FOSL2, NEDD9 (H. Oh, et al., Immunol Rev 252, 41-51 (2013)), ZEB2, BCL2A1 (M. Mandal, et al., J Exp Med 201, 603-614 (2005)), BIRC3 (S. A. Sarkar, et al., Diabetologia 52, 1092-1101 (2009)), DUSP4/MKP-2 (A. Lawan, et al., J Biol Chem 286, 12933-12943 (2011); M. S. Al-Mutairi, et al., PLoS Pathog 6, e1001192 (2010)), CFLAR (cFLIP/CASPER) (V. Tseveleki, et al., J Immunol 173, 6619-6626 (2004)). Together, these expression patterns suggest that these cells are endowed with properties that allow them to exert sustained and strong type 2 inflammatory responses in asthma.

IL-9-Expressing HDM-Reactive T_{H}2 Cells are Increased in Asthma

It was next sought to identify potential asthma-specific changes in HDM-reactive T_{H}2 cells from subjects with HDM-allergy. Single-cell differential gene expression analysis of HDM-reactive T_{H}2 cells from HDM-allergic asthmatics versus non-asthmatics revealed that among the T_{H}2-enriched transcripts, IL9 was the most upregulated gene in asthmatics subjects (data not shown). Furthermore, sub-clustering of the T_{H}2 subset showed that IL9-expressing cells were highly enriched in the larger T_{H}2 sub-cluster (data not shown). The relative proportion of cells in the IL9-enriched T_{H}2 subset (T_{H}2-cluster 1) was greater in asthmatics compared with (C. Micosse, et al., Sci Immunol 4 (2019)) non-asthmatic subjects (data not shown). Thus, enrichment of IL9 expression in T_{H}2 cells appears to be associated in the development of asthma.

To determine the properties of IL9-expressing HDM-reactive T_{H}2 cells in asthmatic subjects, the single-cell gene expression profiles of IL9-expressing and non-expressing cells contained within the IL9-enriched T_{H}2-cluster 1 were compared. Surprisingly, the expression of several transcripts encoding products linked to pathogenicity and survival of T_{H}2 cells was increased in IL9-expressing cells (Table 1). These included transcripts encoding canonical T_{H}2 cytokine IL-5, cytotoxicity molecules (granzyme B, ZEB2, EFHD2), T_{H}2 polarizing and survival-related signaling receptor (IL-33R)(M. Lohning, et al., Proc Natl Acad Sci USA 95, 6930-6935 (1998); F. Alvarez, et al., Front Immunol 10, 522 (2019)), and CD109, a membrane-anchored molecule described as negative regulator of TGFβ signaling (K. W. Finnson, et al., FASEB J 20, 1525-1527 (2006); G. L. Stritesky, et al., Immunity 34, 39-49 (2011)) but also as a co-activator of the JAK/STAT3 signaling pathway (G. L. Stritesky, et al., Immunity 34, 39-49 (2011); C. H. Chuang, et al., Nat Med 23, 291-300 (2017); I. V. Litvinov, et al., Exp Dermatol 20, 627-632 (2011)) that is important for T_{H}2 cell development (G. L. Stritesky, et al., Immunity 34, 39-49 (2011); A. C. Gavino, et al., Allergy 71, 1684-1692 (2016)) (Table 1). Overall, these findings suggest that IL9-expressing HDM-reactive T_{H}2 cells displayed greater pathogenic properties that could play an important role in driving asthma pathogenesis.

Materials and Methods

Study design. The goal of this study was to use bulk and single-cell RNA-seq assay to capture the transcriptome of HDM allergen-reactive CD4⁺ T cell memory subsets from peripheral blood mononuclear cells (PBMC) of 6 asthmatic subjects allergic to HDM, 6 asthmatic subjects non-allergic to HDM, 6 non-asthmatic subjects allergic to HDM, and 6 non-asthmatic non-allergic to HDM. To isolate HDM-reactive CD4⁺ cells, PBMC were stimulated with HDM and CD4⁺ memory cells were sorted based on CD154 and CD137 expression: CD154⁺ (HDM-reactive T_{H}), CD154⁻ CD137⁺ (HDM-reactive T_{reg}), and CD154⁻ CD137⁻ (HDM-non-reactive T cells). For bulk RNA-seq, 200 cells were collected in triplicates, and for single-cell RNA-seq assay, between 1,500 to 2,000 cells per cell type per patient were collected.

Subject recruitment, ethical approval and characteristics. Recruitment of subjects included in this study followed Institutional Review Board (La Jolla Institute for Immunology, La Jolla, Calif.) approval, and study participants gave written informed consent. Twelve non-smoking subjects with mild asthma treated only with inhaled bronchodilators (mild asthma), six subjects with allergic rhinitis but no asthma, meeting established diagnostic criteria, and 6 healthy nonatopic subjects were studied (data not shown). Subjects with asthma underwent pulmonary function tests and/or methacholine challenge to establish diagnosis (bronchodilator response of 0.12%, or 0.200 ml, and/or methacholine challenge with a provocative concentration causing a drop of the forced expiratory volume in 1 s [FEV1] of 20%, 8 mg/ml). All subjects were classified as allergic to HDM based on skin test reactivity to HDM allergens (Der p and Der f).

Sample processing. Peripheral blood mononuclear cells (PBMC) obtained from blood samples by density gradient centrifugation according to the manufacturer's instructions and cryopreserved in liquid nitrogen.

Antigen-specific T cell enrichment (ARTS) assay. The HDM peptide pool was generated as described and contained 75 peptides at a total concentration of 20 mg/ml (188.7 ng/ml for each peptide) (D. Hinz, et al., Clin Exp Allergy 45, 1601-1612 (2015); C. Oseroff, et al., Clin Exp Allergy 47, 577-592 (2017)). The assay to isolate HDM-reactive T cells based on HDM peptide pool stimulation, MACS-based enrichment and FACS sorting of CD154⁺ memory CD4⁺ T cells from PBMC was adapted from Bacher et al. 2016 (P. Bacher, et al., Cell 167, 1067-1078 e1016 (2016)). For each donor, PBMC cryovials were thawed, washed and, plated overnight in 6-well culture plates at a concentration of 10×10⁶ cells/ml in 2 ml of serum-free TexMACS medium (Miltenyi Biotec) (5% CO₂, 37° C.). In presence of a blocking CD40 antibody (1 μg/ml; Miltenyi Biotec), cells were then stimulated by addition of HDM peptide pool (1 μg/ml; methods) for 6 h. Subsequently, cells were stained with fluorescence-labeled antibodies and a biotinylated CD154 antibody (clone 5C8; Miltenyi Biotec). Anti-biotin microbeads (Miltenyi Biotec) were added to allow MACS-based enrichment of CD154⁺ cells using MS columns (Miltenyi Biotec). 5% of cells were kept as control sample (‘input’) and used for FACS sorting of HDM⁻ T cells and analysis of cell frequencies before enrichment. Positively selected cells (CD154^{k}) were eluted from the column and used for FACS sorting of CD154⁺ memory CD4⁺ T cells. The flow-through from the MACS column was collected, stained with a biotinylated CD137 antibody (clone REA765; Miltenyi Biotec) and anti-biotin MicroBeads and applied to a second MS column. Positively selected cells (CD137⁺) were used for FACS sorting of CD137⁺ cells. All cell populations were FACS-sorted using a FACSAria-II (Becton Dickinson); the gating strategy is not shown. All flow cytometry data were analyzed using FlowJo software (version 10).

Cell isolation for bulk and single-cell RNA-seq assay. For bulk assays, cells of interest were directly collected by sorting 200 cells into 0.2 ml PCR tubes (low-retention, Axygen) containing 8 μl of ice-cold lysis buffer (Triton X-100 [0.1%, Sigma-Aldrich] containing RNase inhibitor (1:100, Takara)). Once collected, tubes were vortexed for 10 seconds, spun for 1 minute at 3000 g and stored at −80° C. For single cell RNA-seq assays (10× Genomics), 1000 to 2000 HDM-reactive T cells per subject were collected by sorting in low retention and sterile ice-cold 1.5 mL collection tubes containing 500 μL of PBS:FBS (1:1 vol:vol) with RNAse inhibitor (1:100). HDM-reactive T cells from 6 subjects in each of the 4 groups (asthma with HDM-allergy, asthma without HDM allergy, HDM-allergy without asthma and healthy without HDM-allergy) were collected in the same tube. Collection tubes with 9,000 to 12,000 sorted cells/study group were inverted a few times, ice-cold PBS was added to reach a volume of 1400 μl, and centrifuged for 5 minutes at a speed of 600 g at 4° C. Supernatant was cautiously removed leaving 5 to 10 μl of volume. Pellets were then resuspended with 25 μl of 10× Genomics resuspension buffer (0.22 μm filtered ice-cold PBS supplemented with ultra-pure bovine serum albumin (0.04%, Sigma-Aldrich)). 33 μl of cell suspension were transferred to an 8 PCR-tube strip for downstream steps as per manufacturer's instructions (10× Genomics, San Francisco).

Bulk RNA library preparation for sequencing. For full-length bulk transcriptome analyses, the Smart-Seq2 protocol (S. Picelli, et al., Nat Methods 10, 1096-1098 (2013)) was used, adapted for samples with small cell numbers (S. L. Rosales, et al., Methods Mol Biol 1799, 275-301 (2018); I. Engel, et al., Nat Immunol 17, 728-739 (2016)). The protocol was performed as described previously (S. L. Rosales, et al., Methods Mol Biol 1799, 275-301 (2018); I. Engel, et al., Nat Immunol 17, 728-739 (2016)) with following modifications: (i) the pre-amplification PCR cycle for T cells was set at 22 cycles; (ii) to eliminate any traces of primer-dimers, the PCR pre-amplified cDNA product was purified using 0.8× Ampure-XP beads (Beckman Coulter) before using the DNA for sequencing library preparation. One ng of pre-amplified cDNA was used to generate barcoded Illumina sequencing libraries (Nextera XT library preparation kit, Illumina) in 8 μl reaction volume. Samples failing any quality control step (DNA quality assessed by capillary electrophoresis (Fragment analyzer, Advance analytical) and quantity (Picogreen quantification assay, Thermofisher)) were eliminated from further downstream steps. Libraries were then pooled at equal molar concentration and sequenced using the HiSeq 2500 Illumina platform to obtain 50-bp single-end reads (HiSeq SBS Kit v4; Illumina). In total, 1.7 billion uniquely mapped reads were generated with a median±standard deviation of 17.8±3.8 million uniquely mapped reads per sample.

10× Genomics single-cell RNA library preparation for sequencing. Samples were processed using 10× Genomics 3′ TAG v2 chemistry as per manufacturer's recommendations; 11 cycles were used for cDNA amplification and library preparation respectively (V. S. Patil, et al., Sci Immunol 3, (2018)). Barcoded RNA was collected and processed following manufacturer's recommendations. After quantification, equal molar concentration of each libraries was pooled and sequenced using the HiSeq2500 Illumina sequencing platform to obtain 26- and 100-bp paired-end reads using the following read length: read 1, 26 cycles; read 2, 100 cycles; and i7 index, 8 cycles.

Bulk RNA-seq analysis. Bulk RNA-seq data were mapped against the hg19 reference using TopHat (C. Trapnell, et al., Bioinformatics 25, 1105-1111 (2009)) (v1.2.1 (--library-type fr-unstranded --no-coverage-search) with FastQC (v0.11.2), Bowtie (B. Langmead, et al., Genome Biol 10, R25 (2009))(v1.1.2), Samtools 0.1.18.0) (H. Li, et al., Bioinformatics 25, 1754-1760 (2009)) and htseq-count -m union -s no -t exon -i gene_name (part of the HTSeq framework, version 0.7.1) was employed (P. Langfelder, et al., BMC Bioinformatics 9, 559 (2008); S. Anders, et al., Bioinformatics 31, 166-169 (2015)). Cutadapt (v1.3) was used to remove adapters (M. Martin, EMBnet.Journal 17, 10-12 (2011)). Values throughout are displayed as log₂ TPM (transcripts per million) counts; a value of 1 was added prior to log transformation (pseudo-count). Principal component analysis and clustering analysis was performed using t-distributed stochastic neighbor embedding dimensional reduction algorithm (tSNE) (L. van der Maaten, Journal of Machine Learning Research 15, 3221-3245 (2014)) (based on 3 PC using the top 200 most variable genes). To identify genes expressed differentially between groups, negative binomial tests were performed for paired comparisons by employing DESeq2 (M. I. Love, et al., Genome Biol 15, 550 (2014)) (1.16.1) with default parameters. Genes were considered expressed differentially by any comparison when the DESeq2 analysis resulted in a Benjamini-Hochberg-adjusted P-value of at most 0.01 and a log₂ fold change of at least 2. Gene set enrichment analysis (GSEA) were performed as previously described (I. Engel, et al., Nat Immunol 17, 728-739 (2016); A. P. Ganesan, et al., Nat Immunol 18, 940-950 (2017)) using the Qlucore visualization software (version 3.5)(A. Subramanian, et al., Proc Natl Acad Sci USA 102, 15545-15550 (2005)).

Single-cell RNA-Seq analysis. Analysis of 3′ single-cell transcriptomes using the 10× Genomics platform. Raw data was processed as previously described (J. Clarke, et al., J Exp Med 216, 2128-2149 (2019); V. S. Patil, et al., Sci Immunol 3, (2018)), merging multiple sequencing runs using count function from Cell Ranger (Table 1), then aggREG ating multiple cell types with cell ranger aggr (v3.0.2). The merged data was transferred to the R statistical environment for analysis using the package Seurat (v3.0.2) (T. Stuart, et al., Cell 177, 1888-1902 e1821 (2019)).

Doublet cell filtering. Barcoded single-cell RNA-seq was demultiplexed patient-wise using Demuxlet (H. M. Kang, et al., Nat Biotechnol 36, 89-94 (2018)) with the following parameters: alpha=0, 0.5 and --geno-error=0.05. Cells called as doublet by Demuxlet were removed from downstream analyses (FIG. 7A and Table 1). Identities were inferred by analyzing VCF files from the genotyping analysis containing the corresponding individual for each particular library. Each cell was assigned a donor ID or marked as a doublet, and then incorporated to the annotation table. There was no observation of major changes in singlets/doublets proportion between the different 10× Genomics libraries (reflecting cell type and subject groups), suggesting optimal processing of cells during 10× (Gel Bead-In Emulsions) GEM generation and downstream steps (FIG. 7A). All downstream analyses were performed using singlet cells.

Activation score and cell filtering. To filter out cells with low level of activation or no activation by the HDM-peptide pool i.e., HDM non-reactive cells, pair-wise single-cell differential gene expression analysis was performed using MAST algorithm (G. Finak, et al., Genome Biol 16, 278 (2015)) between HDM-reactive T_{H} cells (CD154⁺ cells, N=3075, random sampled) and HDM⁻ T cells (CD154^{neg} cells, N=3075). A gene set was defined (N=110 genes, called HDM⁺ T_{H} activation genes) that captured the transcripts upregulated in the CD154⁺ versus CD154^{neg} cells (Table 1) using the following parameters (FIG. 7B): Benjamini-Hochberg-adjusted P-value ≤0.05, log₂ fold change ≥2, log₂ mean of expression ≥0.75 CPM and, percentage of expressing cells (>0 CPM) in HDM⁺ T_{H} cells >37.5% (FIG. 7B). Each cell was scored using AddModuleScore from Seurat (T. Stuart, et al., Cell 177, 1888-1902 e1821 (2019)). Briefly, the module score is calculated by binning the genes by the average expression level, then the average expression of each gene is subtracted by the aggregated expression of the control gene sets (100) randomly selected per bin. Finally, based on the distribution of cells based on their activation score (FIG. 7C), a threshold was applied for defining activated cells. The proportions of cells expressing important canonical genes such as IL4, IL5, IFNG, IL17A pre- and post-activation filtering indicated cell eliminated due to low-activation score did not upregulate transcripts for these genes (data not shown). Similarly, an independent HDM⁺ T_{reg} activation score was also calculated using similar approach to analyze HDM⁺ T_{reg} single-cell datasets (Table 1 and FIG. 7, right panels). In total, 3505 cells with low-activation score were eliminated from downstream analysis.

Transcriptome-based clustering analysis. The merged data was transferred to the R (v3.5) statistical environment for analysis using the package Seurat (v3.0.2)(T. Stuart, et al., Cell 177, 1888-1902 e1821 (2019)). Only cells expressing more than 200 genes and with a total mitochondrial gene expression less than 5%, and genes expressed in at least 3 cells were included in the analysis. The data was then log-normalized per cell and a list of the most variable genes with a mean expression >0.1 (UMI) and explaining 30% of the cumulative standardized variance given by the Find VariableFeatures function were used for clustering analysis (data not shown). The clustering analysis was performed using distinct lists of most variable genes for HDM⁺ T_{H}, HDM⁺ T_{REG} and HDM⁺ T_{H}2 clusters (Table 1). In regard to T_{H}2 sub-clustering the most variable genes that were expressed were done so by more than 10% of the cells and with a standardized variance greater than 2. The selection of the most variable genes was limited to 10% of the cumulative standardized variance (data not shown). Normalized single-cell transcriptomic data was then further scaled by the number of UMI-detected and percentage of mitochondrial reads. Principal component (PC) analysis was performed with RunPCA algorithm (T. Stuart, et al., Cell 177, 1888-1902 e1821 (2019)) using the determined most variable gene lists. Seurat procedure was used to determine the number of PCs to select for downstream analyses, using the standard deviation of PCs. FindNeighbors and FindClusters functions from Seurat were applied with default settings (Table 1) to identify clusters. All clusters had more than 50 cells and none were excluded from the downstream analysis. Cluster specific markers were obtained by the FindAllMarkers function with default parameters, test.use=MAST (G. Finak, et al., Genome Biol 16, 278 (2015)). Further visualizations of exported normalized data such has “violin” plots were generated using the Seurat package and custom R scripts. Notably, the violin plots show Seurat normalized expression for a particular gene (log₂ (CPM+1 pseudo-count)) only for the cells expressing the gene of interest. Violin shape represents the distribution of cell expressing transcript of interest (based on a Gaussian Kernel density estimation model) and are marked according to the percentage of cell expressing the transcript of interest.

Single-cell differential gene expression analysis. Pairwise single-cell differential gene expression analysis was conducted after conversion of data to count per million base-pairs (CPM+1) using MAST algorithm (q<0.05, v1.2.1) (R package) (G. Finak, et al., Genome Biol 16, 278 (2015)). Equal number of cells from each subject group were used, and random sampling performed when necessary. A gene was considered differentially expressed when Benjamini-Hochberg-adjusted P-value was <0.05 and a log₂ fold change was more than 0.25. For cluster-specific signatures, a gene was considered significantly different (unique to a group), only if the gene was enriched in every pair-wise comparison for a particular cluster with other clusters.

Single-cell co-expression analysis and weighted gene correlation analysis (WGCNA). In order to perform co-expression analysis, given the high levels of genes drop-out associated with single cell analysis, a data imputation was performed using SAVER imputation algorithm (M. Huang, et al., Nat Methods 15, 539-542 (2018)). Briefly, SAVER analysis was implemented on the Cell Ranger UMI matrix output for HDM⁺ T_{H} using the function saver (v1.1.1) with pred.genes.only=TRUE. Then, Spearman correlations coefficients were calculated using the cor function and determined the cluster-modules through hclust on Euclidean distances and cutree functions k=5 according to the within groups sums of squares elbow (similar to T_{H} single-cell clustering analysis). Weighted correlation analysis was performedusing WGCNA algorithm (v1.66) (P. Langfelder, et al., BMC Bioinformatics 9, 559 (2008)) using the function TOMsimilarityfromExpr, power=3, and exportNetworkToCytoscape, weighted=TRUE, threshold=50^{th} quantile of the topological overlap matrix. Network plots were generated by Gephi (0.9.2) using Fruchterman Reingold and Noverlap layouts (M. Bastian, et al., Gephi: An Open Source Software for Exploring and Manipulating Networks. 2009 (2009)). The size and color of the nodes were defined according to the degree, while the edge width and color were scaled according to the weight value.

Genotyping. For each patient, genomic DNA was isolated from PBMC using the DNeasy Blood and Tissue Kit (Qiagen) and utilized for genotyping using the Infinium Multi-Ethnic Global-8 Kit (Illumina) following the manufacturer's instructions. Raw data from the genotyping analysis, data quality assessment and SNPs identification were performed as previously described (B. J. Schmiedel, et al., Cell 175, 1701-1715 e1716 (2018)).

Stimulation of memory CD4+ T cells with human recombinant TRAIL. Memory CD4⁺ T cells were isolated from PBMC using the ‘Memory CD4⁺ T Cell Isolation Kit’ (Miltenyi Biotec) and cultured in Iscove's Modified Dulbecco's Medium (IMDM; Invitrogen) supplemented with 5% (vol/vol) heat-inactivated fetal bovine serum (FBS) and 2% (vol/vol) human AB serum (CellGro). The memory CD4⁺ T cells were stimulated with pre-coated human recombinant TRAIL (5 μg/ml), anti-CD3 antibodies (2.5 μg/ml) and soluble anti-CD28 antibodies (1 μg/ml) in presence of IL-7 (5 ng/ml; Miltenyi Biotec). The expression of surface markers (CD69, CD154, CD137) was analyzed by flow cytometry after 6 h.

Expression of TRAIL on memory CD4+ T cells. Total PBMC were thawed, washed and plated overnight in serum-free TexMACS medium (Miltenyi Biotec)/complete IMDM (as described above). In presence of a blocking CD40 antibody (1 μg/ml in culture; clone HB14; Miltenyi Biotec), cells were then left untreated or stimulated by addition of the control reagent CytoStim (1:500 dilution of stock; Miltenyi Biotec). The expression of surface markers (CD69, CD154 and TRAIL) was analyzed by FACS after 6 h.

Statistical analysis. Non-parametric Kruskal-Wallis one-way analysis of variance test (ANOVA) was used to compare unpaired data for more than 2 conditions and Kolmogoroz-Smirnov test when comparing 2 groups of data. Paired t-Student test was used for time-course flow cytometry analysis. Additionally, GraphPad Prism 7.0 was used.

TABLE 1
Differentially expressed genes and statistics for TH clusters

| | | | | Heatmap | | |
|---|---|---|---|---|---|---|
| | | | | | | Minimum fold |
| | Comparison Cluster vs all other clusters | | | | | change's |
| | Log Fold | Adjusted | | | | adjusted |
| Gene ID | Change | P-value | Cluster | Group | Rank | p-value |
| IL13 | 3.8848 | 0 | TH2 | TH2 | 1 | 0 |
| IL4 | 3.9036 | 0 | TH2 | TH2 | 2 | 0 |
| IL31 | 3.0596 | 0 | TH2 | TH2 | 3 | 1.6192E−290 |
| IL1RL1 | 2.3626 | 0 | TH2 | TH2 | 4 | 1.9507E−288 |
| IL5 | 6.082 | 0 | TH2 | TH2 | 5 | 5.2155E−162 |
| PLA2G16 | 0.9653 | 0 | TH2 | TH2 | 6 | 5.5155E−123 |
| SRGN | 0.3027 & | 0 & | TH1 & | TH2 | 7 | 2.5877E−121 |
| | 1.0746 | 0 | TH2 | | | |
| ICOS | 0.9899 | 0 | TH2 | TH2 | 8 | 8.6568E−112 |
| GATA3 | 0.9698 | 0 | TH2 | TH2 | 9 | 2.0837E−110 |
| RAD50 | 0.9692 | 0 | TH2 | TH2 | 10 | 4.2682E−104 |
| GK | 0.9841 | 0 | TH2 | TH2 | 11 | 2.00198E−97 |
| MAP3K8 | 0.9032 | 0 | TH2 | TH2 | 12 | 2.72002E−84 |
| CTLA4 | 1.0763 | 0 | TH2 | TH2 | 13 | 2.21347E−74 |
| ARL5B | 0.8596 | 0 | TH2 | TH2 | 14 | 1.80706E−64 |
| CD109 | 0.7165 | 0 | TH2 | TH2 | 15 | 7.92203E−61 |
| CREM | 0.8435 | 0 | TH2 | TH2 | 16 | 1.55842E−59 |
| RARRES3 | 0.9943 | 0 | TH2 | TH2 | 17 | 2.15818E−59 |
| TPRG1 | 0.5543 | 0 | TH2 | TH2 | 18 | 1.50533E−52 |
| SDCBP | 0.4294 & | 0 & | TH1 & | TH2 | 19 | 4.4515E−45 |
| | 0.9864 | 0 | TH2 | | | |
| THADA | 0.455 | 0 | TH2 | TH2 | 20 | 4.91891E−38 |
| IL17RB | NA | NA | NA | TH2 | 21 | 1.66377E−36 |
| TNFRSF9 | 0.3046 & | 0 & | ACT3 & | TH2 | 22 | 1.05271E−35 |
| | 0.3582 & | 0 & | TH1 & | | | |
| | 0.7304 | 0 | TH2 | | | |
| DUSP6 | 0.4199 | 0 | TH2 | TH2 | 23 | 1.41574E−35 |
| CMC2 | 0.2533 & | 0 & | TH1 & | TH2 | 24 | 7.95199E−35 |
| | 0.7132 | 0 | TH2 | | | |
| IL9 | 4.1012 | 0 | TH2 | TH2 | 25 | 1.37653E−34 |
| GADD45G | 0.666 | 0 | TH2 | TH2 | 26 | 1.58736E−34 |
| TGFBR3 | 0.3909 | 0 | TH2 | TH2 | 27 | 8.55033E−31 |
| TNFSF8 | 0.6862 | 0 | TH2 | TH2 | 28 | 1.49399E−29 |
| NR3C1 | 0.6951 | 0 | TH2 | TH2 | 29 | 4.03897E−29 |
| FFAR3 | NA | NA | NA | TH2 | 30 | 2.37055E−28 |
| CLECL1 | 0.6367 | 0 | TH2 | TH2 | 31 | 6.65476E−28 |
| SERP1 | 0.478 | 0 | TH2 | TH2 | 32 | 5.04055E−27 |
| LIMA1 | 0.4689 | 0 | TH2 | TH2 | 33 | 1.52582E−25 |
| TNFSF14 | 0.7468 & | 0 & | TH1 & | TH2 | 34 | 1.57451E−25 |
| | 1.1283 | 0 | TH2 | | | |
| AHI1 | 0.594 | 0 | TH2 | TH2 | 35 | 4.52344E−25 |
| UFM1 | 0.3343 & | 0 & | TH1 & | TH2 | 36 | 2.29991E−24 |
| | 0.6459 | 0 | TH2 | | | |
| IL21 | 1.1816 | 0 | TH2 | TH2 | 37 | 4.09894E−24 |
| BCL2A1 | 0.5182 & | 0 & | ACT3 & | TH2 | 38 | 6.79625E−24 |
| | 0.6073 & | 0 & | TH1 & | | | |
| | 0.9216 | 0 | TH2 | | | |
| SELK | 0.8829 | 0 | TH2 | TH2 | 39 | 8.56809E−24 |
| SOD1 | 0.4356 | 0 | TH2 | TH2 | 40 | 1.56741E−23 |
| HLA-DRB1 | 0.4709 | 0 | TH2 | TH2 | 41 | 4.93405E−23 |
| RAB27A | 0.6043 | 0 | TH2 | TH2 | 42 | 1.03721E−22 |
| LINC00152 | 0.4624 & | 0 & | TH1 & | TH2 | 43 | 8.05955E−22 |
| | 0.8437 | 0 | TH2 | | | |
| CD82 | 0.5255 & | 0 & | TH1 & | TH2 | 44 | 1.35629E−21 |
| | 0.814 | 0 | TH2 | | | |
| NAMPT | 0.2531 & | 0 & | ACT3 & | TH2 | 45 | 2.14459E−21 |
| | 0.3043 & | 0 & | TH1 & | | | |
| | 0.9565 & | 0 & | TH2 & | | | |
| | 0.3691 | 0 | TH17 | | | |
| PHLDA1 | 0.3474 & | 0 & | TH1 & | TH2 | 46 | 6.73731E−21 |
| | 0.8285 | 0 | TH2 | | | |
| RBM8A | 0.2816 & | 0 & | TH1 & | TH2 | 47 | 9.87735E−21 |
| | 0.5082 | 0 | TH2 | | | |
| PKIA | 0.2871 & | 0 & | TH1 & | TH2 | 48 | 1.25203E−20 |
| | 0.6772 | 0 | TH2 | | | |
| PTGS2 | NA | NA | NA | TH2 | 49 | 3.22057E−20 |
| TRAF3 | 0.4517 | 0 | TH2 | TH2 | 50 | 1.90638E−19 |
| C10orf54 | 0.7686 & | 0 & | TH1 & | TH2 | 51 | 2.67804E−19 |
| | 1.0739 | 0 | TH2 | | | |
| ACSL4 | 0.4073 | 0 | TH2 | TH2 | 52 | 3.41666E−19 |
| EVIS | NA | NA | NA | TH2 | 53 | 4.17491E−19 |
| ATP1B3 | 0.4379 | 0 | TH2 | TH2 | 54 | 7.69077E−19 |
| FDX1 | 0.569 | 0 | TH2 | TH2 | 55 | 8.18162E−19 |
| PHF6 | 0.3879 & | 0 & | TH1 & | TH2 | 56 | 2.99177E−18 |
| | 0.6919 | 0 | TH2 | | | |
| DUSP4 | 0.4722 & | 0 & | TH1 & | TH2 | 57 | 6.35419E−18 |
| | 0.7471 | 0 | TH2 | | | |
| GPR35 | NA | NA | NA | TH2 | 58 | 2.80465E−17 |
| HCST | 0.659 & | 0 & | TH1 & | TH2 | 59 | 7.88281E−17 |
| | 0.9731 | 0 | TH2 | | | |
| PRDX1 | 0.5356 | 0 | TH2 | TH2 | 60 | 1.41716E−16 |
| SIAH2 | 0.6069 | 0 | TH2 | TH2 | 61 | 1.71973E−16 |
| GATA3-AS1 | 0.4616 | 0 | TH2 | TH2 | 62 | 3.85646E−16 |
| SLAMF1 | 0.3518 & | 0 & | TH1 & | TH2 | 63 | 4.41342E−16 |
| | 0.6986 & | 0 & | TH2 & | | | |
| | 0.2921 | 0 | TH17 | | | |
| TFRC | 0.4713 | 0 | TH2 | TH2 | 64 | 1.14582E−15 |
| AIM1 | 0.3342 | 0 | TH2 | TH2 | 65 | 1.6904E−15 |
| CACNA1D | NA | NA | NA | TH2 | 66 | 2.52811E−15 |
| FKBP1A | 0.5037 | 0 | TH2 | TH2 | 67 | 5.37296E−15 |
| GPR42 | NA | NA | NA | TH2 | 68 | 1.1489E−14 |
| CMSS1 | 0.4769 | 0 | TH2 | TH2 | 69 | 1.62357E−14 |
| CHDH | NA | NA | NA | TH2 | 70 | 1.87336E−14 |
| MAPK1 | NA | NA | NA | TH2 | 71 | 3.43134E−14 |
| IRF4 | 0.4598 | 0 | TH2 | TH2 | 72 | 4.50927E−14 |
| SELT | 0.3751 | 0 | TH2 | TH2 | 73 | 4.64512E−14 |
| NUSAP1 | NA | NA | NA | TH2 | 74 | 5.72526E−14 |
| PRKCA | NA | NA | NA | TH2 | 75 | 6.44122E−14 |
| TIGIT | 0.4043 | 0 | TH2 | TH2 | 76 | 7.14138E−14 |
| IL3 | 2.0429 | 0 | TH2 | TH2 | 77 | 8.07327E−14 |
| HLA-E | 0.3567 | 0 | TH2 | TH2 | 78 | 8.76286E−14 |
| JOSD1 | 0.3024 | 0 | TH2 | TH2 | 79 | 4.58547E−13 |
| RNF19A | 0.9407 & | 0 & | TH1 & | TH2 | 80 | 1.18406E−12 |
| | 1.0928 | 0 | TH2 | | | |
| ATL3 | 0.3454 | 0 | TH2 | TH2 | 81 | 1.18406E−12 |
| IQGAP2 | NA | NA | NA | TH2 | 82 | 1.28937E−12 |
| PTTG1 | 0.4779 | 0 | TH2 | TH2 | 83 | 1.72372E−12 |
| NFKBID | 0.7629 & | 0 & | TH1 & | TH2 | 84 | 1.83325E−12 |
| | 1.0231 | 0 | TH2 | | | |
| PSMA2.1 | 0.2624 & | 0 & | TH1 & | TH2 | 85 | 2.13645E−12 |
| | 0.4484 | 0 | TH2 | | | |
| PTPLA | NA | NA | NA | TH2 | 86 | 6.64852E−12 |
| HLA-DPB1 | NA | NA | NA | TH2 | 87 | 7.30684E−12 |
| RAB8B | 0.2516 & | 0 & | ACT3 & | TH2 | 88 | 1.15744E−11 |
| | 0.2563 & | 0 & | TH1 & | | | |
| | 0.5285 | 0 | TH2 | | | |
| TOP1 | 0.374 | 0 | TH2 | TH2 | 89 | 2.3043E−11 |
| ITGAV | NA | NA | NA | TH2 | 90 | 4.07784E−11 |
| H3F3B | 0.2979 & | 0 & | TH1 & | TH2 | 91 | 5.33058E−11 |
| | 0.4661 | 0 | TH2 | | | |
| HRASLS5 | NA | NA | NA | TH2 | 92 | 6.9481E−11 |
| SMAP2 | 0.3052 | 0 | TH2 | TH2 | 93 | 2.23964E−10 |
| RUNX3 | 0.2581 & | 0 & | TH1 & | TH2 | 94 | 2.68254E−10 |
| | 0.4645 | 0 | TH2 | | | |
| CASC7 | 0.2976 & | 0 & | TH1 & | TH2 | 95 | 2.84234E−10 |
| | 0.4961 | 0 | TH2 | | | |
| GPR171 | 0.7933 | 0 | TH2 | TH2 | 96 | 6.15157E−10 |
| ERN1 | NA | NA | NA | TH2 | 97 | 8.07144E−10 |
| PTGER4 | 0.6858 | 0 | TH2 | TH2 | 98 | 1.06414E−09 |
| ZC3H12C | NA | NA | NA | TH2 | 99 | 1.59951E−09 |
| ARFGAP3 | 0.2727 | 0 | TH2 | TH2 | 100 | 1.77143E−09 |
| SLC27A2 | NA | NA | NA | TH2 | 101 | 2.74986E−09 |
| RERE | NA | NA | NA | TH2 | 102 | 3.35923E−09 |
| GRB2 | NA | NA | NA | TH2 | 103 | 3.81765E−09 |
| KLHL6 | NA | NA | NA | TH2 | 104 | 6.20388E−09 |
| ARMCX3 | 0.3018 | 0 | TH2 | TH2 | 105 | 9.00651E−09 |
| IFNAR2 | 0.3447 | 0 | TH2 | TH2 | 106 | 9.14265E−09 |
| MTHFD2 | 0.2776 & | 0 & | ACT3 & | TH2 | 107 | 9.58041E−09 |
| | 0.2653 & | 0 & | TH1 & | | | |
| | 0.3931 | 0 | TH2 | | | |
| B3GNT4 | NA | NA | NA | TH2 | 108 | 1.31578E−08 |
| NIPA1 | NA | NA | NA | TH2 | 109 | 1.33079E−08 |
| RP11-141M1.3 | NA | NA | NA | TH2 | 110 | 1.48495E−08 |
| EXOC2 | NA | NA | NA | TH2 | 111 | 2.08697E−08 |
| LAIR2 | NA | NA | NA | TH2 | 112 | 5.14458E−08 |
| AP3M2 | 0.2884 | 0 | TH2 | TH2 | 113 | 1.05591E−07 |
| TIAM1 | 0.2793 | 0 | TH2 | TH2 | 114 | 1.20797E−07 |
| ATP6V1D | NA | NA | NA | TH2 | 115 | 1.3627E−07 |
| SLC4A7 | 0.3812 | 0 | TH2 | TH2 | 116 | 1.59913E−07 |
| ZC2HC1A | NA | NA | NA | TH2 | 117 | 1.96862E−07 |
| DUSP18 | NA | NA | NA | TH2 | 118 | 2.49895E−07 |
| CRADD | 0.6756 | 0 | TH2 | TH2 | 119 | 4.74205E−07 |
| GCNT1 | NA | NA | NA | TH2 | 120 | 7.32662E−07 |
| NR4A3 | 0.4741 & | 0 & | TH1 & | TH2 | 121 | 8.28369E−07 |
| | 0.6123 | 0 | TH2 | | | |
| GABPB1 | 0.364 | 0 | TH2 | TH2 | 122 | 1.34261E−06 |
| POU2F2 | 0.2713 | 0 | TH2 | TH2 | 123 | 1.42441E−06 |
| FOSL2 | 0.402 | 0 | TH2 | TH2 | 124 | 1.54793E−06 |
| GFI1 | NA | NA | NA | TH2 | 125 | 1.57101E−06 |
| F5 | 0.3093 | 0 | TH2 | TH2 | 126 | 2.31066E−06 |
| UBE2S | NA | NA | NA | TH2 | 127 | 2.43186E−06 |
| FRMD4B | 0.4525 | 0 | TH2 | TH2 | 128 | 2.84845E−06 |
| VDR | NA | NA | NA | TH2 | 129 | 2.96716E−06 |
| SLA | 0.6308 & | 0 & | TH1 & | TH2 | 130 | 4.56122E−06 |
| | 0.7273 | 0 | TH2 | | | |
| THAP2 | 0.3684 | 0 | TH2 | TH2 | 131 | 5.08714E−06 |
| B3GNT5 | NA | NA | NA | TH2 | 132 | 6.48598E−06 |
| DDX3Y | NA | NA | NA | TH2 | 133 | 6.76932E−06 |
| NAA50 | 0.2725 | 0 | TH2 | TH2 | 134 | 6.90704E−06 |
| MT-CO1 | NA | NA | NA | TH2 | 135 | 6.95359E−06 |
| PDCD1 | 0.372 | 0 | TH2 | TH2 | 136 | 8.58861E−06 |
| NIPA2 | 0.2945 | 0 | TH2 | TH2 | 137 | 1.01161E−05 |
| PIM3 | 0.4082 & | 0 & | ACT3 & | TH2 | 138 | 1.088E−05 |
| | 0.9096 & | 0 & | TH1 & | | | |
| | 0.736 | 0 | TH2 | | | |
| INPP4B | NA | NA | NA | TH2 | 139 | 1.21091E−05 |
| PPARG | 0.4572 | 0 | TH2 | TH2 | 140 | 1.51001E−05 |
| RNMT | 0.3457 | 0 | TH2 | TH2 | 141 | 3.19881E−05 |
| SLC2A3 | 0.3878 | 0 | TH2 | TH2 | 142 | 3.76262E−05 |
| CFLAR | 0.4554 | 0 | TH2 | TH2 | 143 | 3.99597E−05 |
| PRNP | 0.4543 | 0 | TH2 | TH2 | 144 | 5.91117E−05 |
| AIM2 | NA | NA | NA | TH2 | 145 | 6.45994E−05 |
| PARK7 | NA | NA | NA | TH2 | 146 | 7.50263E−05 |
| SP140 | 0.2542 | 0 | TH2 | TH2 | 147 | 8.1748E−05 |
| KIAA0513 | NA | NA | NA | TH2 | 148 | 9.36144E−05 |
| H3F3A | 0.2801 | 0 | TH2 | TH2 | 149 | 9.58854E−05 |
| TNFSF11 | NA | NA | NA | TH2 | 150 | 0.000102567 |
| TBL1X | 0.2771 | 0 | TH2 | TH2 | 151 | 0.000113983 |
| VMA21 | 0.2692 | 0 | TH2 | TH2 | 152 | 0.00013077 |
| TSPAN13 | NA | NA | NA | TH2 | 153 | 0.000138202 |
| ZEB2 | 0.7229 & | 0 & | TH2 & | TH2 | 154 | 0.000148558 |
| | 0.4138 | 0 | TH17 | | | |
| NEDD9 | 0.2582 & | 0 & | TH1 & | TH2 | 155 | 0.000153185 |
| | 0.3942 | 0 | TH2 | | | |
| SBDS | 0.2915 | 0 | TH2 | TH2 | 156 | 0.000173075 |
| MRPL32 | 0.3863 | 0 | TH2 | TH2 | 157 | 0.000182139 |
| SFT2D2 | NA | NA | NA | TH2 | 158 | 0.000197302 |
| CD58 | 0.3492 | 0 | TH2 | TH2 | 159 | 0.000208393 |
| RP11-379F12.4 | NA | NA | NA | TH2 | 160 | 0.000244325 |
| C12orf75 | NA | NA | NA | TH2 | 161 | 0.000336357 |
| AC002331.1 | NA | NA | NA | TH2 | 162 | 0.000341836 |
| ATP13A3 | NA | NA | NA | TH2 | 163 | 0.000371552 |
| DR1 | NA | NA | NA | TH2 | 164 | 0.000469033 |
| KCNK5 | NA | NA | NA | TH2 | 165 | 0.000546079 |
| POLR2K | 0.4041 & | 0 & | TH1 & | TH2 | 166 | 0.000584274 |
| | 0.4981 | 0 | TH2 | | | |
| NQO2 | NA | NA | NA | TH2 | 167 | 0.000629841 |
| MORF4L2 | NA | NA | NA | TH2 | 168 | 0.000660523 |
| MAPKAPK2 | NA | NA | NA | TH2 | 169 | 0.000680031 |
| RPS6KA3 | NA | NA | NA | TH2 | 170 | 0.000689576 |
| PSMD13 | 0.2615 & | 0 & | TH1 & | TH2 | 171 | 0.000709867 |
| | 0.3641 | 0 | TH2 | | | |
| CHSY1 | NA | NA | NA | TH2 | 172 | 0.000801728 |
| TIMM8A | 0.2608 | 0 | TH2 | TH2 | 173 | 0.000823805 |
| SLC35E4 | NA | NA | NA | TH2 | 174 | 0.000891197 |
| IL10 | 1.6938 | 0 | TH2 | TH2 | 175 | 0.001171793 |
| RBBP8 | 0.3932 | 0 | TH2 | TH2 | 176 | 0.001171954 |
| FYN | 0.4522 & | 0 & | TH1 & | TH2 | 177 | 0.001261689 |
| | 0.5044 | 0 | TH2 | | | |
| FAM107B | 0.4763 | 0 | TH2 | TH2 | 178 | 0.001354304 |
| HPGDS | NA | NA | NA | TH2 | 179 | 0.001467291 |
| MAPK6 | 0.3725 | 0 | TH2 | TH2 | 180 | 0.001518197 |
| EIF5B | 0.2776 | 0 | TH2 | TH2 | 181 | 0.001546237 |
| HOTAIRM1 | NA | NA | NA | TH2 | 182 | 0.001665137 |
| PPP1R2 | 0.5942 & | 0 & | TH1 & | TH2 | 183 | 0.001751137 |
| | 0.5553 | 0 | TH2 | | | |
| DYNLT3 | NA | NA | NA | TH2 | 184 | 0.001786494 |
| KDM6B | 0.3163 & | 0 & | TH1 & | TH2 | 185 | 0.001845406 |
| | 0.4217 | 0 | TH2 | | | |
| FGL2 | NA | NA | NA | TH2 | 186 | 0.001924747 |
| UHRF1BP1L | NA | NA | NA | TH2 | 187 | 0.002281786 |
| FBXW11 | NA | NA | NA | TH2 | 188 | 0.002542695 |
| CALM3 | NA | NA | NA | TH2 | 189 | 0.002636748 |
| NDFIP2 | 0.4756 & | 0 & | TH1 & | TH2 | 190 | 0.002817783 |
| | 0.5915 | 0 | TH2 | | | |
| KIF3A | NA | NA | NA | TH2 | 191 | 0.002834727 |
| CSF2 | 1.5225 & | 0 & | TH1 & | TH2 | 192 | 0.002853855 |
| | 1.6796 & | 0 & | TH2 & | | | |
| | 0.7375 | 0 | TH17 | | | |
| SPCS3 | 0.2868 | 0 | TH2 | TH2 | 193 | 0.003097823 |
| PBX4 | 0.2564 | 0 | TH2 | TH2 | 194 | 0.003143947 |
| HSP90AB1 | NA | NA | NA | TH2 | 195 | 0.003486315 |
| PTPN11 | NA | NA | NA | TH2 | 196 | 0.003539482 |
| TRAM1 | NA | NA | NA | TH2 | 197 | 0.003684115 |
| NEK6 | NA | NA | NA | TH2 | 198 | 0.003740355 |
| SMIM4 | 0.2586 | 0 | TH2 | TH2 | 199 | 0.003763795 |
| CD28 | NA | NA | NA | TH2 | 200 | 0.004004217 |
| TMEM2 | NA | NA | NA | TH2 | 201 | 0.004163689 |
| MRPS6 | 0.2924 | 0 | TH2 | TH2 | 202 | 0.004671151 |
| EIF3J | 0.2802 & | 0 & | ACT3 & | TH2 | 203 | 0.004677822 |
| | 0.3142 | 0 | TH2 | | | |
| POLB | 0.3367 | 0 | TH2 | TH2 | 204 | 0.005081292 |
| LIF | NA | NA | NA | TH2 | 205 | 0.005260532 |
| IL24 | NA | NA | NA | TH2 | 206 | 0.005814744 |
| OAZ1 | NA | NA | NA | TH2 | 207 | 0.006624909 |
| PLIN2 | 0.3471 | 0 | TH2 | TH2 | 208 | 0.006993723 |
| TBC1D4 | 0.3694 | 0 | TH2 | TH2 | 209 | 0.00700071 |
| SACS | 0.2578 | 0 | TH2 | TH2 | 210 | 0.008063862 |
| GPN2 | NA | NA | NA | TH2 | 211 | 0.008771622 |
| TET2 | NA | NA | NA | TH2 | 212 | 0.010035339 |
| BACH1 | NA | NA | NA | TH2 | 213 | 0.010394447 |
| SATB1 | NA | NA | NA | TH2 | 214 | 0.011742268 |
| GZMB | NA | NA | NA | TH2 | 215 | 0.013679687 |
| CHD1 | NA | NA | NA | TH2 | 216 | 0.013697127 |
| STAT4 | 0.2538 | 0 | TH2 | TH2 | 217 | 0.014473273 |
| PDLIM5 | NA | NA | NA | TH2 | 218 | 0.014821433 |
| ZC3H8 | 0.268 | 0 | TH2 | TH2 | 219 | 0.016131727 |
| SEC61B | 0.3479 | 0 | TH2 | TH2 | 220 | 0.016315101 |
| SDC4 | 0.3594 | 0 | TH2 | TH2 | 221 | 0.017067862 |
| BTLA | 0.2628 | 0 | TH2 | TH2 | 222 | 0.017532341 |
| ZBTB32 | 0.5471 & | 0 & | TH2 & | TH2 | 223 | 0.017584397 |
| | 0.3029 | 0 | TH17 | | | |
| RIOK1 | NA | NA | NA | TH2 | 224 | 0.018088086 |
| BIRC3 | 0.5786 & | 0 & | ACT3 & | TH2 | 225 | 0.021901144 |
| | 0.4332 & | 0 & | TH1 & | | | |
| | 0.5941 | 0 | TH2 | | | |
| ST8SIA4 | 0.3336 | 0 | TH2 | TH2 | 226 | 0.022664618 |
| SUGT1 | 0.3198 | 0 | TH2 | TH2 | 227 | 0.025931759 |
| RHOH | NA | NA | NA | TH2 | 228 | 0.02754583 |
| PFDN2 | 0.3889 & | 0 & | ACT3 & | TH2 | 229 | 0.02948613 |
| | 0.2864 & | 0 & | TH1 & | | | |
| | 0.3708 | 0 | TH2 | | | |
| BLOC1S2 | NA | NA | NA | TH2 | 230 | 0.030503885 |
| CTA-29F11.1 | NA | NA | NA | TH2 | 231 | 0.03129747 |
| HRH1 | NA | NA | NA | TH2 | 232 | 0.035738879 |
| MCOLN2 | 0.2558 | 0 | TH2 | TH2 | 233 | 0.035738879 |
| NOP10 | 0.2578 & | 0 & | TH1 & | TH2 | 234 | 0.036954604 |
| | 0.2717 | 0 | TH2 | | | |
| DDX24 | NA | NA | NA | TH2 | 235 | 0.037874001 |
| VMP1 | NA | NA | NA | TH2 | 236 | 0.04366083 |
| TNIK | NA | NA | NA | TH2 | 237 | 0.043815338 |
| GNL3 | NA | NA | NA | TH2 | 238 | 0.044302508 |
| PIGT | NA | NA | NA | TH2 | 239 | 0.046974547 |
| PURB | NA | NA | NA | TH2 | 240 | 0.047917958 |
| CD52 | 0.275 & | 0 & | ACT2 & | TH1 | 1 | 4.21983E−36 |
| | 0.6402 | 0 | TH1 | | | |
| RGS16 | 0.4199 & | 0 & | ACT3 & | TH1 | 2 | 3.09883E−29 |
| | 1.0155 | 0 | TH1 | | | |
| CD27 | 0.5241 | 0 | TH1 | TH1 | 3 | 5.63326E−24 |
| ZBED2 | 0.5598 | 0 | TH1 | TH1 | 4 | 8.68096E−17 |
| TSHZ2 | 0.4747 | 0 | TH1 | TH1 | 5 | 3.46368E−16 |
| TAGAP | 0.4304 | 0 | TH1 | TH1 | 6 | 5.3092E−16 |
| ID3 | 0.5582 | 0 | TH1 | TH1 | 7 | 5.50554E−15 |
| GAPDH | 0.5927 | 0 | TH1 | TH1 | 8 | 1.71504E−14 |
| TGFBR2 | NA | NA | NA | TH1 | 9 | 9.61008E−14 |
| XCL2 | NA | NA | NA | TH1 | 10 | 1.35421E−12 |
| APOBEC3G | 0.3712 | 0 | TH1 | TH1 | 11 | 3.88414E−12 |
| CD3D | 0.3623 | 0 | TH1 | TH1 | 12 | 6.27444E−11 |
| IFNG | 2.9731 | 0 | TH1 | TH1 | 13 | 7.03473E−11 |
| CD3E | 0.2528 | 0 | TH1 | TH1 | 14 | 7.76682E−11 |
| C1orf228 | NA | NA | NA | TH1 | 15 | 1.3252E−10 |
| IL7R | 0.4869 | 0 | TH1 | TH1 | 16 | 3.04932E−10 |
| MSMO1 | NA | NA | NA | TH1 | 17 | 2.6728E−07 |
| CD84 | 0.3841 | 0 | TH1 | TH1 | 18 | 8.6909E−07 |
| AC013264.2 | NA | NA | NA | TH1 | 19 | 1.81669E−06 |
| DUSP2 | 0.453 & | 0 & | ACT3 & | TH1 | 20 | 1.9053E−06 |
| | 0.8383 & | 0 & | TH1 & | | | |
| | 0.5106 | 0 | TH2 | | | |
| SERPINE2 | NA | NA | NA | TH1 | 21 | 2.46521E−06 |
| XCL1 | NA | NA | NA | TH1 | 22 | 3.34578E−06 |
| JUND | 0.3551 | 0 | TH1 | TH1 | 23 | 5.22326E−06 |
| PRF1 | NA | NA | NA | TH1 | 24 | 8.34905E−06 |
| C12orf57 | NA | NA | NA | TH1 | 25 | 1.08225E−05 |
| SCML1 | NA | NA | NA | TH1 | 26 | 1.71721E−05 |
| CD226 | 0.2782 | 0 | TH1 | TH1 | 27 | 2.04169E−05 |
| COX7A2 | NA | NA | NA | TH1 | 28 | 6.68002E−05 |
| ALDOA | 0.2648 | 0 | TH1 | TH1 | 29 | 9.19579E−05 |
| DDX5 | NA | NA | NA | TH1 | 30 | 0.000132455 |
| APOBEC3C | NA | NA | NA | TH1 | 31 | 0.00015244 |
| CD69 | 0.9865 & | 0 & | TH1 & | TH1 | 32 | 0.000428672 |
| | 0.6839 | 0 | TH2 | | | |
| CXCR3 | NA | NA | NA | TH1 | 33 | 0.000445346 |
| NFATC1 | NA | NA | NA | TH1 | 34 | 0.000899351 |
| RGS10 | 0.3863 | 0 | TH1 | TH1 | 35 | 0.001139175 |
| ARHGEF3 | NA | NA | NA | TH1 | 36 | 0.001679606 |
| FASLG | 0.4849 | 0 | TH1 | TH1 | 37 | 0.001784797 |
| RGCC | 1.4151 & | 0 & | TH1 & | TH1 | 38 | 0.001817534 |
| | 1.0465 | 0 | TH2 | | | |
| PTPN7 | 0.3466 | 0 | TH1 | TH1 | 39 | 0.002018267 |
| KLRG1 | NA | NA | NA | TH1 | 40 | 0.002023879 |
| ARHGEF1 | NA | NA | NA | TH1 | 41 | 0.002060018 |
| BMI1 | NA | NA | NA | TH1 | 42 | 0.002371872 |
| PTMA | NA | NA | NA | TH1 | 43 | 0.002643295 |
| SLC25A5 | NA | NA | NA | TH1 | 44 | 0.002643927 |
| FBLN7 | NA | NA | NA | TH1 | 45 | 0.003043968 |
| BTG1 | 0.2893 & | 0 & | ACT3 & | TH1 | 46 | 0.003554612 |
| | 0.3341 | 0 | TH1 | | | |
| SNX9 | NA | NA | NA | TH1 | 47 | 0.004133905 |
| CD40LG | 0.7695 & | 0 & | TH1 & | TH1 | 48 | 0.004734422 |
| | 0.7872 & | 0 & | TH2 & | | | |
| | 0.2644 | 0 | TH17 | | | |
| CDC42EP3 | NA | NA | NA | TH1 | 49 | 0.005511721 |
| PTPN22 | 0.5527 & | 0 & | TH1 & | TH1 | 50 | 0.006626666 |
| | 0.3105 | 0 | TH2 | | | |
| TPST2 | NA | NA | NA | TH1 | 51 | 0.010684195 |
| SEMA7A | NA | NA | NA | TH1 | 52 | 0.014239714 |
| CD200 | 1.0562 & | 0 & | TH1 & | TH1 | 53 | 0.014572524 |
| | 0.6846 | 0 | TH2 | | | |
| LTBP4 | NA | NA | NA | TH1 | 54 | 0.015837887 |
| SNRPD2 | NA | NA | NA | TH1 | 55 | 0.021026892 |
| ATP6V1G1 | NA | NA | NA | TH1 | 56 | 0.022516548 |
| UCP2 | NA | NA | NA | TH1 | 57 | 0.023307805 |
| HRH2 | NA | NA | NA | TH1 | 58 | 0.026037854 |
| CD97 | 0.2919 | 0 | TH1 | TH1 | 59 | 0.027318222 |
| GADD45A | 0.2813 & | 0 & | ACT3 & | TH1 | 60 | 0.031988571 |
| | 0.4252 | 0 | TH1 | | | |
| EEMO1 | NA | NA | NA | TH1 | 61 | 0.039622755 |
| BEX2 | NA | NA | NA | TH1 | 62 | 0.039819059 |
| LSM4 | NA | NA | NA | TH1 | 63 | 0.047454552 |
| CD2 | NA | NA | NA | TH1 | 64 | 0.049373322 |
| IL17F | 4.7538 | 0 | TH17 | TH17 | 1 | 1.621E−141 |
| IL17A | 4.63 | 0 | TH17 | TH17 | 2 | 4.4083E−138 |
| CCL20 | 1.4417 & | 0 & | ACT3 & | TH17 | 3 | 1.95981E−23 |
| | 0.6342 & | 0 & | TH1 & | | | |
| | 1.659 | 0 | TH17 | | | |
| CTSH | 0.4354 & | 0 & | ACT2 & | TH17 | 4 | 1.48231E−08 |
| | 0.8981 | | TH17 | | | |
| IL22 | 2.1328 | 0 | TH17 | TH17 | 5 | 2.34946E−06 |
| MSC | 0.6309 | 0 | TH17 | TH17 | 6 | 3.6822E−06 |
| AC092580.4 | 1.1839 | 0 | TH17 | TH17 | 7 | 7.15633E−06 |
| CCR6 | 0.3053 & | 0 & | ACT2 & | TH17 | 8 | 2.92962E−05 |
| | 0.7008 | 0 | TH17 | | | |
| IQCG | 0.4646 | 0 | TH17 | TH17 | 9 | 0.000205155 |
| MACC1 | NA | NA | NA | TH17 | 10 | 0.000582344 |
| IL2RA | 0.532 & | 0 & | ACT2 & | TH17 | 11 | 0.003574409 |
| | 0.3538 & | 0 & | ACT3 & | | | |
| | 0.5108 | 0 | TH17 | | | |
| BLM | 0.4426 | 0 | TH17 | TH17 | 12 | 0.00442416 |
| PTPN4 | NA | NA | NA | TH17 | 13 | 0.023358956 |
| NTRK2 | 0.3486 | 0 | TH17 | TH17 | 14 | 0.038905525 |
| MX1 | 1.7729 | 0 | THIFNR | THIFNR | 1 | 0 |
| IFI6 | 2.3187 | 0 | THIFNR | THIFNR | 2 | 0 |
| ISG15 | 2.2117 | 0 | THIFNR | THIFNR | 3 | 0 |
| IFI44L | 1.6839 | 0 | THIFNR | THIFNR | 4 | 4.9414E−303 |
| ISG20 | 1.5731 | 0 | THIFNR | THIFNR | 5 | 2.1729E−232 |
| IFIT3 | 1.2313 | 0 | THIFNR | THIFNR | 6 | 2.2274E−149 |
| LY6E | 1.1856 | 0 | THIFNR | THIFNR | 7 | 9.692E−146 |
| MX2 | 1.185 | 0 | THIFNR | THIFNR | 8 | 4.0834E−138 |
| IFITM1 | 1.1001 | 0 | THIFNR | THIFNR | 9 | 2.9789E−112 |
| EIF2AK2 | 0.9951 | 0 | THIFNR | THIFNR | 10 | 1.00451E−95 |
| SAT1 | 1.5356 | 0 | THIFNR | THIFNR | 11 | 1.52492E−94 |
| XAF1 | 0.983 | 0 | THIFNR | THIFNR | 12 | 2.72792E−86 |
| OAS1 | 0.7593 | 0 | THIFNR | THIFNR | 13 | 1.02783E−85 |
| OAS3 | 0.9362 | 0 | THIFNR | THIFNR | 14 | 4.63309E−77 |
| IFIT1 | 0.6729 | 0 | THIFNR | THIFNR | 15 | 1.0982E−75 |
| HERC5 | 0.9032 | 0 | THIFNR | THIFNR | 16 | 1.40299E−73 |
| EPSTI1 | 1.0362 | 0 | THIFNR | THIFNR | 17 | 4.94024E−65 |
| USP18 | 0.5739 | 0 | THIFNR | THIFNR | 18 | 1.17515E−62 |
| SAMD9 | 0.9651 | 0 | THIFNR | THIFNR | 19 | 1.5497E−59 |
| RSAD2 | 0.5548 | 0 | THIFNR | THIFNR | 20 | 2.11858E−56 |
| PLSCR1 | 0.6735 | 0 | THIFNR | THIFNR | 21 | 5.38077E−56 |
| IRF7 | 0.6857 | 0 | THIFNR | THIFNR | 22 | 7.37072E−55 |
| IFI16 | 0.8746 | 0 | THIFNR | THIFNR | 23 | 8.29059E−55 |
| SP100 | 0.7907 | 0 | THIFNR | THIFNR | 24 | 4.79953E−51 |
| SAMD9L | 0.2537 & | 0 & | ACT2 & | THIFNR | 25 | 8.07362E−48 |
| | 1.0344 | 0 | THIFNR | | | |
| SP110 | 0.709 | 0 | THIFNR | THIFNR | 26 | 9.56639E−45 |
| IFI35 | 0.7576 | 0 | THIFNR | THIFNR | 27 | 1.92829E−43 |
| OAS2 | 0.538 | 0 | THIFNR | THIFNR | 28 | 3.02817E−37 |
| DDX60L | 0.4395 | 0 | THIFNR | THIFNR | 29 | 7.67064E−37 |
| OASL | 0.4663 | 0 | THIFNR | THIFNR | 30 | 2.09733E−35 |
| HERC6 | 0.5875 | 0 | THIFNR | THIFNR | 31 | 4.49484E−35 |
| BST2 | 0.2983 & | 0 & | ACT2& | THIFNR | 32 | 6.90947E−35 |
| | 0.849 | 0 | THIFNR | | | |
| CMPK2 | 0.3482 | 0 | THIFNR | THIFNR | 33 | 2.8911E−33 |
| TRIM22 | 0.7808 | 0 | THIFNR | THIFNR | 34 | 2.92937E−32 |
| IFIT5 | 0.4722 | 0 | THIFNR | THIFNR | 35 | 3.40612E−32 |
| RNF213 | 0.3851 & | 0 & | ACT2 & | THIFNR | 36 | 4.43847E−31 |
| | 0.9896 | 0 | THIFNR | | | |
| STAT1 | 0.674 & | 0 & | ACT2 & | THIFNR | 37 | 7.55105E−29 |
| | 1.0491 | 0 | THIFNR | | | |
| NT5C3A | 0.7003 | 0 | THIFNR | THIFNR | 38 | 7.98939E−28 |
| IFIT2 | 0.4379 | 0 | THIFNR | THIFNR | 39 | 3.26571E−27 |
| DDX60 | 0.4861 | 0 | THIFNR | THIFNR | 40 | 7.38225E−27 |
| PPM1K | 0.6577 | 0 | THIFNR | THIFNR | 41 | 3.64923E−24 |
| CHMP5 | 0.5627 | 0 | THIFNR | THIFNR | 42 | 6.43729E−23 |
| DRAP1 | 0.5001 | 0 | THIFNR | THIFNR | 43 | 3.86961E−21 |
| C19orf66 | 0.5042 | 0 | THIFNR | THIFNR | 44 | 2.36244E−20 |
| SPATS2L | 0.482 | 0 | THIFNR | THIFNR | 45 | 2.68496E−20 |
| IFIH1 | 0.4752 | 0 | THIFNR | THIFNR | 46 | 2.98058E−20 |
| RPL27 | NA | NA | NA | THIFNR | 47 | 3.48012E−18 |
| DDX58 | 0.4289 | 0 | THIFNR | THIFNR | 48 | 1.68755E−17 |
| NUB1 | 0.5279 | 0 | THIFNR | THIFNR | 49 | 5.24313E−17 |
| LAP3 | 0.4773 | 0 | THIFNR | THIFNR | 50 | 9.25962E−17 |
| LGALS9 | NA | NA | NA | THIFNR | 51 | 1.20866E−16 |
| DTX3L | 0.4749 | 0 | THIFNR | THIFNR | 52 | 5.2774E−16 |
| TNFSF13B | NA | NA | NA | THIFNR | 53 | 2.01292E−15 |
| LAMP3 | 0.2592 | 0 | THIFNR | THIFNR | 54 | 2.17486E−15 |
| IFI44 | 0.7675 | 0 | THIFNR | THIFNR | 55 | 3.5814E−15 |
| HELZ2 | 0.3001 | 0 | THIFNR | THIFNR | 56 | 1.04805E−14 |
| PARP9 | 0.498 | 0 | THIFNR | THIFNR | 57 | 1.66295E−14 |
| HSH2D | NA | NA | NA | THIFNR | 58 | 1.9865E−14 |
| NMI | 0.4821 | 0 | THIFNR | THIFNR | 59 | 2.16774E−14 |
| ADAR | 0.56 | 0 | THIFNR | THIFNR | 60 | 3.40199E−14 |
| TNFSF10 | 0.7194 | 0 | THIFNR | THIFNR | 61 | 7.69912E−13 |
| PLAC8 | 0.4307 | 0 | THIFNR | THIFNR | 62 | 2.83581E−12 |
| SLFN5 | 0.4761 | 0 | THIFNR | THIFNR | 63 | 8.2399E−12 |
| PSMB9 | 0.4148 & | 0 & | ACT2 & | THIFNR | 64 | 1.93363E−11 |
| | 0.5265 | 0 | THIFNR | | | |
| NAPA | NA | NA | NA | THIFNR | 65 | 2.4163E−11 |
| SMCHD1 | 0.4973 | 0 | THIFNR | THIFNR | 66 | 3.34046E−11 |
| STAT2 | 0.3081 | 0 | THIFNR | THIFNR | 67 | 1.24073E−10 |
| IFITM3 | 0.3462 | 0 | THIFNR | THIFNR | 68 | 1.5376E−10 |
| PARP14 | 0.3176 & | 0 & | ACT2 & | THIFNR | 69 | 1.75148E−10 |
| | 0.6848 | 0 | THIFNR | | | |
| CD164 | 0.3492 | 0 | THIFNR | THIFNR | 70 | 9.10272E−10 |
| UBE2L6 | 0.2727 & | 0 & | ACT2 & | THIFNR | 71 | 5.81984E−09 |
| | 0.5231 | 0 | THIFNR | | | |
| XRN1 | 0.4573 | 0 | THIFNR | THIFNR | 72 | 1.46457E−08 |
| PARP11 | NA | NA | NA | THIFNR | 73 | 2.71582E−08 |
| TBIM25 | NA | NA | NA | THIFNR | 74 | 3.62361E−08 |
| TMEM123 | 0.3381 | 0 | THIFNR | THIFNR | 75 | 2.89128E−07 |
| CXCL10 | 0.8969 | 0 | THIFNR | THIFNR | 76 | 3.25822E−07 |
| TAP2.1 | NA | NA | NA | THIFNR | 77 | 5.03241E−07 |
| PGAP1 | NA | NA | NA | THIFNR | 78 | 5.42126E−07 |
| PML | NA | NA | NA | THIFNR | 79 | 5.64109E−07 |
| TRANK1 | NA | NA | NA | THIFNR | 80 | 1.2639E−06 |
| B2M | NA | NA | NA | THIFNR | 81 | 1.42585E−06 |
| UTRN | NA | NA | NA | THIFNR | 82 | 1.51971E−06 |
| PCGF5 | NA | NA | NA | THIFNR | 83 | 2.08916E−06 |
| IFITM2 | 0.3853 & | 0 & | TH1 & | THIFNR | 84 | 4.99597E−06 |
| | 0.3884 | 0 | THIFNR | | | |
| BCL2L14 | NA | NA | NA | THIFNR | 85 | 5.8754E−06 |
| ZNFX1 | NA | NA | NA | THIFNR | 86 | 1.72894E−05 |
| PHF11 | 0.3479 | 0 | THIFNR | THIFNR | 87 | 1.97826E−05 |
| PSME2 | 0.3091 & | 0 & | ACT2 & | THIFNR | 88 | 2.24838E−05 |
| | 0.4423 | 0 | THIFNR | | | |
| IRF9 | 0.372 | 0 | THIFNR | THIFNR | 89 | 2.38402E−05 |
| TRIM38 | NA | NA | NA | THIFNR | 90 | 0.000138496 |
| NEXN | NA | NA | NA | THIFNR | 91 | 0.000159283 |
| LGALS3BP | NA | NA | NA | THIFNR | 92 | 0.000161904 |
| TREX1 | NA | NA | NA | THIFNR | 93 | 0.000188849 |
| EMR1 | NA | NA | NA | THIFNR | 94 | 0.000215617 |
| RBCK1 | NA | NA | NA | THIFNR | 95 | 0.000240042 |
| PSMB8 | 0.3139 & | 0 & | ACT2 & | THIFNR | 96 | 0.000258696 |
| | 0.4054 | 0 | THIFNR | | | |
| TYMP | 0.4552 & | 0 & | ACT2 & | THIFNR | 97 | 0.000490476 |
| | 0.597 | 0 | THIFNR | | | |
| C5orf56 | 0.2588 | 0 | THIFNR | THIFNR | 98 | 0.000633146 |
| HSPB1 | NA | NA | NA | THIFNR | 99 | 0.000660961 |
| HLA-C | 0.2923 | 0 | THIFNR | THIFNR | 100 | 0.000824839 |
| APOL6 | 0.2932 & | 0 & | ACT2 & | THIFNR | 101 | 0.001034164 |
| | 0.4934 | 0 | THIFNR | | | |
| LBH | NA | NA | NA | THIFNR | 102 | 0.001158896 |
| DCK | NA | NA | NA | THIFNR | 103 | 0.001251144 |
| PARP12 | NA | NA | NA | THIFNR | 104 | 0.001661888 |
| TMEM140 | NA | NA | NA | THIFNR | 105 | 0.00269824 |
| NCOA3 | NA | NA | NA | THIFNR | 106 | 0.002809824 |
| BBX | NA | NA | NA | THIFNR | 107 | 0.004160711 |
| RTP4 | NA | NA | NA | THIFNR | 108 | 0.004622544 |
| TRIM5 | NA | NA | NA | THIFNR | 109 | 0.004816107 |
| FYTTD1 | NA | NA | NA | THIFNR | 110 | 0.005708154 |
| SUSD3 | NA | NA | NA | THIFNR | 111 | 0.006592773 |
| STYX | NA | NA | NA | THIFNR | 112 | 0.007985824 |
| MALAT1 | NA | NA | NA | THIFNR | 113 | 0.008336263 |
| RDH14 | NA | NA | NA | THIFNR | 114 | 0.009362548 |
| PNPT1 | 0.371 | 0 | THIFNR | THIFNR | 115 | 0.011841875 |
| CLEC2B | NA | NA | NA | THIFNR | 116 | 0.013269577 |
| HLA-A | NA | NA | NA | THIFNR | 117 | 0.021013513 |
| UBXN2A | NA | NA | NA | THIFNR | 118 | 0.036013222 |

### Example 2

Increasing prevalence of asthma and allergy—Asthma is a rising health problem with no current curative solutions. Asthma is a non-infectious chronic disease of the airways characterized by recurrent attacks of breathlessness and wheezing. Asthma affects 1 in 12 Americans which represents over 25 million people, 334 million worldwide, making it the most common chronic non-infectious disease (Moodley D, et al., Proc Natl Acad Sci USA. 2016; 113(35):9852-7. Epub 2016 Aug. 16; Vollmer W M, et al., Am J Respir Crit Care Med. 2002; 165(2):195-9. Epub 2002 Jan. 16; Bateman E D, et al., Eur Respir J. 2018; 51(2). Epub 2018 Feb. 2). Patients suffer from poor quality of life and have a high risk of asthma-related ICU hospitalizations, ER visits, or death (Chung K F, et al., Eur Respir J. 2014; 43(2):343-73. Epub 2013 Dec. 18). Furthermore, the prevalence of asthma has been steadily increasing in the US, due to multiple factors such as increased levels of pollution, urbanization, obesity, and climate change (D'Amato G, et al., World Allergy Organ J. 2015; 8(1):25. Epub 2015 Jul. 25; Gold D R, Wright R. Annu Rev Public Health. 2005; 26:89-113. Epub 2005 Mar. 12). Biologically, asthma is a complex disease, with many endotypes (Fahy J V. Nat Rev Immunol. 2015; 15(1):57-65. Epub 2014 Dec. 24; Lambrecht B N, et al., Nat Immunol. 2015; 16(1):45-56. Epub 2014 Dec. 19). The dominant endotype of asthma (T2^{high}) is characterized by the recruitment, accumulation in tissue, and aberrant activation of T_{H}2 cells and eosinophils (Lambrecht B N, et al., Nat Immunol. 2015; 16(1):45-56. Epub 2014 Dec. 19; Akuthota P, et al., Semin Hematol. 2012; 49(2):113-9. Epub 2012 Mar. 28). The alarming epidemiologic statistics coupled with the failure of the current T_{H}2 therapies to control/cure asthma underscores the need to revisit the understanding of immune mechanisms underpinning allergic responses in asthma.

Role of allergen-specific T cells in asthma—Asthma has long been considered to result from aberrant type 2 immune responses to airborne innocuous substances (Lambrecht B N, et al., Nat Immunol. 2015; 16(1):45-56. Epub 2014 Dec 19; Kay A B. N Engl J Med. 2001; 344(1):30-7. Epub 2001 Jan. 4). Indeed, specific allergen sensitization and/or exposure to numerous non-pathogenic antigens such as pollens, house dust mite (HDM) or molds (fungi) has been strongly associated with type 2 responses and asthma-severity-related outcomes (Targonski P V, et al., J Allergy Clin Immunol. 1995; 95(5 Pt 1):955-61. Epub 1995 May 1; Dullaers M, Schuijs M J, et al., J Allergy Clin Immunol. 2017; 140(1):76-88 e7. Epub 2016 Oct. 18; Arshad S H, et al., Am J Respir Crit Care Med. 1998; 157(6 Pt 1):1900-6. Epub 1998 Jun. 25). The hallmarks of asthma, namely airway narrowing and sputum eosinophilia, have been shown to result from the specific activation of (MHC) class II-restricted CD4⁺ helper T cells (T_{H}) by challenging asthmatics with synthetic allergen-derived peptides (Arshad S H, et al., Am J Respir Crit Care Med. 1998; 157(6 Pt 1):1900-6. Epub 1998 Jun. 25; Ali F R, et al., Am J Respir Crit Care Med. 2004; 169(1):20-6. Epub 2003 Sep. 23; Muehling L M, et al., J Allergy Clin Immunol. 2017; 140(6):1523-40. Epub 2017 Apr. 27). Further evidence of the centrality of T_{H} cells in asthma pathology is that their depletion reduces allergic airway inflammation in animal models (Raemdonck K, et al., Respir Res. 2016; 17:45. Epub 2016 Apr. 27), and that inhibition of T_{H} cell-derived type 2 cytokines (IL-5, IL-13, IL-4) is clinically beneficial in patients with asthma (Gibeon D, et al., Expert Rev Respir Med. 2012; 6(4):423-39. Epub 2012 Sep. 14). However, despite the central role of allergen-reactive T_{H} cells and their products in driving airway inflammation, the full spectrum and function of T_{H} cell subsets that respond to common allergens has yet to be defined. Studies of allergen-reactive T cells, such as the one disclosed herein as EXAMPLE 1, have characterized the phenotype based on the expression of cell-surface markers or canonical cytokines (Seumois G, et al., Sci Immunol. 2020; 5(48). Epub 2020 Jun. 14; Wambre E, et al., Sci Transl Med. 2017; 9(401). Epub 2017 Aug. 5; Upadhyaya B, et al., J Immunol. 2011; 187(6):3111-20. Epub 2011 Aug. 19; Smith K A, et al., BMC Immunol. 2013; 14:49. Epub 2013 Nov. 6) and showed presence of other T cell subsets such as T_{H}1, T_{H}17, T_{H}22, T_{H}9 (Durrant D M, et al., Immunol Invest. 2010; 39(4-5):526-49. Epub 2010 May 11; Ling M F, et al., Ann Am Thorac Soc. 2016; 13 Suppl 1:S25-30. Epub 2016 Mar. 31). Furthermore, many studies showed that dysregulated immune response in chronic diseases is often associated with the development of new cell subsets with enhanced pathogenic properties. For instance, T_{H}2 cells with pathogenic properties have been identified from patients with allergy and asthma (Seumois G, et al., J Immunol. 2016; 197(2):655-64. Epub 2016 Jun. 9; Shinoda K, et al., Allergol Int. 2017; 66(3):369-76. Epub 2017/04/11), and the existence of T_{H}2/T_{H}17 (Liu W, et al., J Allergy Clin Immunol. 2017; 139(5):1548-58 e4. Epub 2016 Oct. 6), T_{H}2/T_{H}9 (Micosse C, et al., Sci Immunol. 2019; 4(31). Epub 2019 Jan. 20), “super-pathogenic” IL5^{high} T_{H}2 cells (Upadhyaya B, et al., J Immunol. 2011; 187(6):3111-20. Epub 2011 Aug. 19) has also been reported. Therefore, the investigation and understanding of the heterogeneity of T_{H} cells in allergic asthma remains an important challenge to overcome.

Single-cell analysis of allergen-reactive T cells—Due to the relative rarity, analyses of allergen-specific these cells usually require in vitro expansion, which can alter their molecular properties, thus limiting the value of unbiased transcriptomic studies (Seumois G, et al., J Allergy Clin Immunol. 2019; 144(5):1150-3. Epub 2019 Nov. 11). A recent single-cell analysis of T_{H} cells in a mouse models of allergic airway inflammation revealed substantial heterogeneity, and also identified T_{H} subsets that had not been previously described (Tibbitt C A, et al., Immunity. 2019; 51(1):169-84 e5. Epub 2019 Jun. 25; Rahimi R A, et al., J Exp Med. 2020; 217(9). Epub 2020 Jun. 25). To better understand the diversity of allergen-specific human T cell subsets in allergic asthma, analyzed herein is the single-cell transcriptome of 50,000 HDM-reactive T_{H} cells and regulatory T_{(REG)} cells from asthmatics with HDM allergy and from three control groups: asthmatics without HDM allergy and non-asthmatics with and without HDM allergy (Seumois G, et al., Sci Immunol. 2020; 5(48). Epub 2020 Jun. 14). The analyses as disclosed herein show that HDM-reactive T_{H} and T_{REG} cells are highly heterogeneous, and certain subsets are quantitatively and qualitatively different in subjects with HDM-reactive asthma.

T_{H}IFNR cells, a novel, protective allergen-specific CD4⁺ helper T cell subset—The single-cell study also helped address an important unanswered question in the field of allergy and asthma research. Most fundamentally, exposure to common allergens, such as HDM, is nearly ubiquitous and T_{H} responses to these allergens are seen in both allergic and non-allergic subjects (Sette A, et al., J Allergy Clin Immunol. 2017; 139(3):769-70. Epub 2016 Dec. 21; Hinz D, et al., Clin Exp Allergy. 2016; 46(5):705-19. Epub 2015 Dec. 15)—Why do only some people develop T_{H}2 responses to allergens? By comparing HDM-reactive T cells from asthmatics with and without HDM allergy (T_{H}2 responses versus no T_{H}2 responses), it was found that in subjects without HDM allergy, a subset of T_{H} and T_{REG} cells expressing the interferon response signature genes, called here as T_{H}IFNR cells, was expanded. These cells expressed higher levels of TRAIL, a molecule that can inhibit TCR signaling (Lehnert C, et al., J Immunol. 2014; 193(8):4021-31. Epub 2014 Sep. 14; Chyuan I T, et al., Mucosal Immunol. 2019; 12(4):980-9. Epub 2019 May 12; Chyuan I T, et al., Cell Mol Immunol. 2018; 15(9):846-57. Epub 2017/04/11), activation of T_{H} cells and inflammation in model systems. Therefore, without wishing to be bound by the theory, it is hypothesized that these TRAIL-expressing HDM-reactive T cells could play an important role in dampening T_{H}2 inflammation in allergy and asthma. This finding implies that T_{H}IFNR cells can be generated and sustained in vivo, and that HDM sensitization of mice may be an appropriate system to test the role of these cells in allergic inflammation as described in this application.

T_{H}IFNR cells are a stable and ubiquitous population of CD4⁺ helper T cells in the blood and lungs—Analysis of the single-cell RNA-seq data sets obtained as disclosed herein or published previously show that T_{H}IFNR cells reactive to other allergens (Aspergillus) and respiratory viruses (SARS-CoV2, influenza, para influenza, metapneumovirus) (Meckiff B J, et al., Single-Cell Transcriptomic Analysis of SARS-CoV-2 Reactive CD4 (+) T Cells. SSRN. 2020:3641939. Epub 2020 Aug. 4) are present in healthy and infected subjects. Furthermore, it was found that T_{H}IFNR cells with tissue-residency features are present in the airways/lungs of healthy and asthmatics subjects (Example 1 and Seumois et al.). Finally, in a large-scale study of 91 healthy subjects, it was found that T_{H}IFNR cells are a stable population of CD4⁺ memory T cells suggesting a potentially broader roles in host immune responses.

Interferons in allergy and asthma—Up to 80% of asthma “attacks”, called exacerbation episodes, are caused by respiratory viral infections (Jartti T, et al., Semin Immunopathol. 2020; 42(1):61-74. Epub 2020 Jan. 29; Busse W W, et al., Lancet. 2010; 376(9743):826-34. Epub 2010 Sep. 8). Defects in the production of type I and III interferons by airway epithelial cells from asthmatic patients has been linked to increased risk of viral exacerbations (Contoli M, et al., Nat Med. 2006; 12(9):1023-6. Epub 2006 Aug. 15). Indeed, interferons (IFN-α/β (McNab F, et al., Nat Rev Immunol. 2015; 15(2):87-103. Epub 2015 Jan. 24), IFN-γ and IFN-λ (Der S D, et al., Proc Natl Acad Sci USA. 1998; 95(26):15623-8. Epub 1998 Dec. 23)), have a hallmark function of antiviral activity, and a multifaceted role in modulating the innate and adaptive defense (Takaoka A, et al., Cell Microbiol. 2006; 8(6):907-22. Epub 2006 May 10; Schreiber G, et al., Trends Immunol. 2015; 36(3):139-49. Epub 2015 Feb. 18). In allergic diseases, multiple human and murine studies have shown that decreased production of IFNs (IFN-β and IFN-λ) and an apparent defective anti-viral IFN response leads to recurrent viral infections (Contoli M, et al., Nat Med. 2006; 12(9):1023-6. Epub 2006 Aug. 15; Wark P A, et al., J Exp Med. 2005; 201(6):937-47. Epub 2005 Mar. 23; Spann K M, et al., Thorax. 2014; 69(10):918-25. Epub 2014 May 9; Lynch J P, et al., J Allergy Clin Immunol. 2016; 138(5):1326-37. Epub 2016 May 30). Those studies also suggest that the apparent deficiency in anti-viral immunity is due to a dominant T_{H}2 inflammatory response (Contoli M, et al., Nat Med. 2006; 12(9):1023-6. Epub 2006 Aug. 15; Wark P A, et al., J Exp Med. 2005; 201(6):937-47. Epub 2005 Mar. 23; Spann K M, et al., Thorax. 2014; 69(10):918-25. Epub 2014 May 9; Lynch J P, et al., J Allergy Clin Immunol. 2016; 138(5):1326-37. Epub 2016 May 30). However, it is conceivable that defects in IFN response may also lead to a failure in the generation the sufficient numbers of T_{H}IFNR cells and resulting unrestrained T_{H}2 cell development. Thus, studies were required to understand the T cell intrinsic role of IFNs and T_{H}IFNR cells in the context of allergy and asthma pathogenesis, which is addressed in this EXAMPLE 2.

In this EXAMPLE 2, the origin, phenotype, and function of a subset of CD4⁺ T cells, the T_{H}IFNR cells are fully characterized.

Building on the discovery of a CD4⁺ T cell subset from single-cell transcriptomic studies of allergen-specific T cells as disclosed in EXAMPLE 1. Key findings are summarized below:

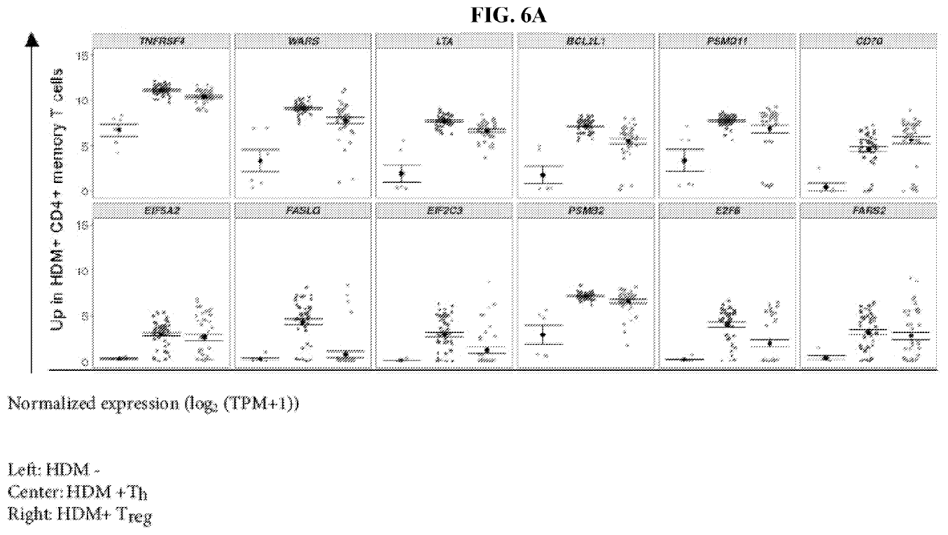

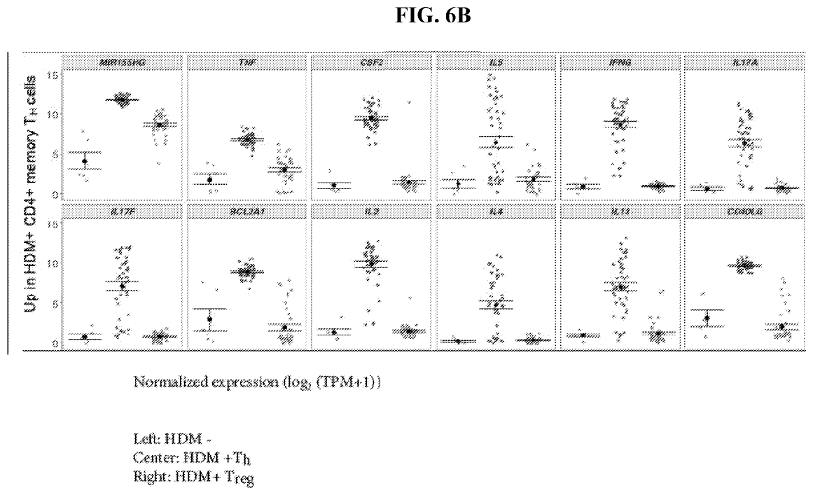

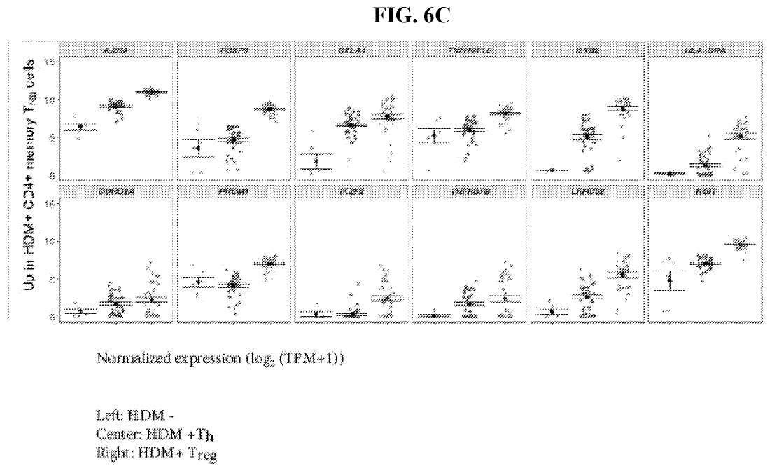

T_{H}IFNR cells, a protective allergen-specific CD4⁺ T cell subset. CD4⁺ helper T cells (T_{H}) and regulatory T cells (T_{REG}) that respond to common allergens play an important role in driving and dampening airway inflammation in patients with asthma. Until recently, direct, unbiased molecular analysis of allergen-reactive T_{H} and T_{REG} cells has not been possible. To better understand the diversity of these T cell subsets in allergy and asthma, the single-cell transcriptome was analyzed of 50,000 house dust mite (HDM) allergen-reactive T_{H} cells and T_{REG} cells from asthmatics with HDM allergy and from three control groups: asthmatics without HDM allergy and non-asthmatics with and without HDM allergy (FIGS. 1-10). Among multiple distinct clusters including T_{H}1, T_{H}2, and T_{H}17 cells, inventors identified a novel subset of HDM-reactive T cells characterized by an interferon response (IFNR) gene signature. The subset of T cells characterized by the IFNR gene signature is referred to herein as T_{H}IFNR cells. It was found that HDM allergen-reactive T_{H} cells are highly heterogeneous, and certain subsets are quantitatively and qualitatively different in subjects with HDM-reactive asthma. Most importantly, it was found that proportions of HDM-reactive T_{H}IFNR cells are increased in asthmatics without HDM allergy compared with those with HDM allergy. T_{H}IFNR cells expressed high levels of TNFSF10, which encodes for TRAIL (Beyer K, et al., Cancers (Basel). 2019; 11(8). Epub 2019 Aug. 16). It was shown that recombinant TRAIL dampens activation of human T_{H} cells. T_{H}IFNR T cells play an important role through TRAIL engagement in dampening T_{H}2 inflammation in allergy and asthma.

T_{H}IFNR cells, a stable and ubiquitous population (FIGS. 11-14). Analysis of single-cell RNA-seq data sets (Seumois G, et al., Sci Immunol. 2020; 5(48). Epub 2020 Jun. 14; Meckiff B J, et al. SSRN. 2020:3641939. Epub 2020 Aug. 4; EXAMPLE 1; Tibbitt C A, et al., Immunity. 2019; 51(1):169-84 e5. Epub 2019 Jun. 25.) show that T_{H}IFNR cells reactive to other allergens and respiratory viruses are present in healthy and infected subjects. Furthermore, it was found that T_{H}IFNR cells with tissue-residency features are present in the airways/lungs of healthy and asthmatics subjects. Finally, in a large-scale study of 91 healthy subjects, it was found that T_{H}IFNR cells are a stable population of CD4⁺ memory T cells, indicating broader roles in host immune responses. In this example, the origins, nature, and function of a novel subset of CD4⁺ T cells, the T_{H}IFNR cells, are fully characterized in the context of allergic airway inflammation in asthma models.

1: The functional role of T_{H}IFNR cells in vivo—Interferon-stimulated response element (ISRE) reporter mice (Uccellini M B, et al., Cell Rep. 2018; 25(10):2784-96 e3. Epub 2018 Dec. 6; Stifter S A, et al., Cell Rep. 2019; 29(11):3539-50 e4. Epub 2019 Dec. 12) and T cell-specific interferon receptor 1 (IFNAR1) knockout mice (Muller U, et al., Science. 1994; 264(5167):1918-21. Epub 1994 Jun. 24) are utilized to determine the importance of T_{H}IFNR cells in controlling allergic airway inflammation in asthma models.

(A) Characteristics of T_{H}IFNR cells— ISRE reporter mice are utilized to define the functional characteristics of T_{H}IFNR cells generated in vivo in an ‘asthma-inflammation model’ (standard HDM-sensitized/challenge (Debeuf N, et al., Curr Protoc Mouse Biol. 2016; 6(2):169-84. Epub 2016 Jun. 2; Nieuwenhuizen N E, et al., J Allergy Clin Immunol. 2012; 130(3):743-50 e8. Epub 2012 May 4; Haspeslagh E, et al., Methods Mol Biol. 2017; 1559:121-36. Epub 2017 Jan. 8)) and ‘asthma-protection model’ (HDM+LPS sensitized/challenge (Bachus H, et al., Immunity. 2019; 50(1):225-40 e4. Epub 2019 Jan. 13; Schuijs M J, et al., Science. 2015; 349(6252):1106-10. Epub 2015 Sep. 5)). The association of the abundance is determined with the degree of allergic inflammation observed in different types of asthma models.

(B) Function of T_{H}IFNR cells—The role of T_{H}IFNR cells is tested in inhibiting allergic airway inflammation in asthma models. In loss-of-function studies, it is determined if mice selectively lacking T cells with IFN response signaling show increased allergic airway inflammation and T_{H}2 cell in the airways. In gain-of-function studies, it is determined if adoptive transfer of HDM-reactive T_{H}IFNR curtails allergic airway inflammation and T_{H}2 cells.

(C) Mechanism of action of T_{H}IFNR cells—TRAIL-deficient and TRAIL-sufficient T_{H}IFNR cells are adoptively transferred in HDM-induced asthma-inflammation model to determine if TRAIL expression by T_{H}IFNR cells is required for suppressing asthma (Picarda G, et al., J Virol. 2019; 93(16). Epub 2019 May 31; Dufour F, et al., Cell Death Differ. 2017; 24(3):500-10. Epub 2017 Feb. 12; Verma S, et al., PLoS Pathog. 2014; 10(8):e1004268. Epub 2014 Aug. 15; Stacey M A, et al., Cell Host Microbe. 2014; 15(4):471-83. Epub 2014 Apr. 12; Nemcovicova I, et al., PLoS Pathog. 2013; 9(3):e1003224. Epub 2013 Apr. 5; Smith W, et al., Cell Host Microbe. 2013; 13(3):324-35. Epub 2013 Mar. 19; Benedict C A, et al., J Exp Med. 2012; 209(11):1903-6. Epub 2012 Oct. 24). Overall, these studies provide much needed insights into the function and mechanism of action of T_{H}IFNR cells in vivo.

2: Identify transcription factors driving the development and maintenance of human T_{H}IFNR cells—Genome-wide enhancer profiling—The single-cell transcriptomic analyses of human T_{H}IFNR cells in various contexts have confirmed that T_{H}IFNR subset is a stable and ubiquitous CD4⁺ helper T cell population present in both the blood and lungs. Here, the transcription factors that are essential for epigenetically programing the development and maintenance of the CD4⁺ T cell subset are defined. Single-cell ATAC-seq analysis is performed in HDM-reactive T_{H}IFNR cells and non-T_{H}IFNR cells from humans and mice to identify enhancers that are specific to T_{H}IFNR cells. Using PageRank (Yu B, et al., Nat Immunol. 2017; 18(5):573-82. Epub 2017 Mar. 14) analysis of ATAC-seq data, the most critical TFs active at enhancers specific to T_{H}IFNR cells are predicted, and actual DNA binding is experimentally tested by expressed TFs using micro-scaled ChIP assays. Together, these analyses identify species-conserved TFs and enhancers that are likely to play an essential role in the differentiation and function of T_{H}IFNR cells. These predictions are followed up with functional experiments. Candidate TFs are knocked down or overexpressed in T cells and their effects on the differentiation and function of T_{H}IFNR cells is determined in vivo (Schmiedel B J, Singh D, Madrigal A, Valdovino-Gonzalez A G, White B M, et al., Cell. 2018; 175(6):1701-15 e16. Epub 2018 Nov. 20).

Overall, studies described in this disclosure improve the understanding of how T_{H}IFNR cells are generated and how they interact with other CD4⁺ T cells subsets like T_{H}2 cells to curtail allergic airway inflammation in asthma, allergy, and viral disease models.

Novel CD4⁺ T cell subtype—Described herein is the first and most extensive survey of single-cell transcriptomes of allergen-specific CD4⁺ T cells from patients with allergy and asthma (Seumois G, et al.). This unbiased approach led to the discovery of a CD4⁺ T cell subset (T_{H}IFNR) that has the potential to control allergic airway inflammation, asthma, and viral disease.

Novel genetic mouse models— IFN-reporter are employed to characterize the molecular properties of T_{H}IFNR cells in vivo in mouse asthma models. Conditional knock-out mice with T cell-specific deletion of interferon receptors or TRAIL is employed to study the functional role of T_{H}IFNR cells (Uccellini M B, et al., Cell Rep. 2018; 25(10):2784-96 e3. Epub 2018 Dec. 6; Stifter S A, et al., Cell Rep. 2019; 29(11):3539-50 e4. Epub 2019 Dec. 12).

New single-cell “unbiased” technologies—Single-cell genomics, such as transcriptomics and genome-wide enhancer profiling assays (epigenetics) are used to investigate the mechanisms by which T_{H}IFNR cells are generated and function (Seumois G, et al., J Allergy Clin Immunol. 2019; 144(5):1150-3). The micro-scaled and single-cell genome-wide assays are used to overcome the challenges of working with small numbers. The molecular drivers of T_{H}IFNR cell development and function are investigated directly from relevant tissue samples, the best possible approach to understanding the biology of immune cells and diseases.

More details about the approaches and experimental results are provided below.

Presented herein are results that support the rationale for investigating the function of a novel CD4⁺ T cell subset— T_{H}IFNR cells—in controlling pathogenesis of allergic asthma. The T_{H}IFNR population was first discovered by the single-cell RNA-seq analysis of HDM allergen-specific T cells in subjects without HDM-allergy (FIGS. 1-10). This T_{H}IFNR population was also detected among virus-specific T cells (Meckiff B J, et al.), airway T cells from asthmatics, and in the circulation of healthy subjects (FIGS. 11-14). Without wishing to be bound by the theory, based on the molecular properties and the context of the discovery, the T_{H}IFNR population plays an important role in modulating the development of allergen-specific T_{H}2 cells and thus allergic asthma, as well as viral disease.

Allergen-specific T cells in asthma at single-cell resolution. Despite the central role of allergen-reactive CD4⁺ T cells and the products thereof in driving airway inflammation, the full spectrum and function of cells that respond to allergens are still unclear. Although the imbalance between T_{REG} and T_{H} cell responses to specific allergens is often associated with the development of allergic asthma (Tibbitt C A et al., Immunity. 2019; 51(1):169-84 e5. Epub 2019 Jun. 25; Schulten V, et al., J Allergy Clin Immunol. 2018; 141(2):775-7 e6. Epub 2017 May 17; Noval Rivas M, et al., J Allergy Clin Immunol. 2016; 138(3):639-52. Epub 2016 Sep. 7; Lewkowich I P, et al., J Exp Med. 2005; 202(11):1549-61. Epub 2005 Nov. 30; Strickland D H, et al., J Exp Med. 2006; 203(12):2649-60. Epub 2006 Nov. 8), the heterogeneity of allergen-reactive T cells remains understudied (Seumois G, et al., J Allergy Clin Immunol. 2019; 144(5):1150-3. Epub 2019 Nov. 11). An important caveat to the previous studies of antigen-reactive T cells studies is that those cells were characterized based on the expression of cell-surface markers or canonical cytokines (Wambre E, et al., Sci Transl Med. 2017; 9(401). Epub 2017 Aug. 5; Upadhyaya B, et al., J Immunol. 2011; 187(6):3111-20. Epub 2011 Aug. 19; Smith K A, et al., BMC Immunol. 2013; 14:49. Epub 2013 Nov. 6). Indeed, single-cell transcriptomic analysis can help define the molecular properties of allergen-reactive T_{H} cells associated with pathology and assess whether these features are the result of an expansion of a pre-existing population of cells or the result of their aberrant differentiation in response to environmental signals (Geginat J, et al., Front Immunol. 2014; 5:630. Epub 2015 Jan. 8; DuPage M, et al., Nat Rev Immunol. 2016; 16(3):149-63. Epub 2016 Feb. 16). To address the latter issue, the subsets of allergen-reactive T_{H} cells must also be defined in subjects without asthma and allergy. Such allergen-reactive T_{H} cells are present even in non-allergic subjects, although it is not known why or how these cells fail to cause allergic responses (Hinz D, et al., Clin Exp Allergy. 2016; 46(5):705-19. Epub 2015 Dec. 15; Birrueta G, et al., PLoS One. 2018; 13(10):e0204620. Epub 2018 Oct. 12; Schulten V, et al., Front Immunol. 2018; 9:235. Epub 2018 Mar. 1; Akdis M, et al., J Exp Med. 2004; 199(11):1567-75. Epub 2004 Jun. 3). To address these questions in a hypothesis-free manner, performed as described herein is a single-cell transcriptomic analysis of HDM-reactive T_{H} and T_{REG} cells, one of the most common and ubiquitous allergens, and sensitization is associated with both the onset of allergic asthma and its severity (Dullaers M, et al., J Allergy Clin Immunol. 2017; 140(1):76-88 e7. Epub 2016 Oct. 18; Gandhi V D, et al., Curr Allergy Asthma Rep. 2013; 13(3):262-70. Epub 2013 Apr. 16).

HDM-allergen reactive T cells are detected in subjects not allergic to HDM—To comprehensively characterize the molecular properties of allergen-reactive T_{H} and T_{REG} cells from patients with asthma, HDM-reactive memory T_{H} and T_{REG} cells were isolated ex vivo from asthmatics with HDM allergy (N=6) and performed both bulk and single-cell RNA-seq (FIG. 1A). To distinguish the molecular features that are specific to asthma as opposed to HDM allergy, similar assays were performed in HDM-reactive T_{H} and T_{REG} cells isolated from HDM-allergic subjects without asthma (N=6). Because allergen-reactive T cells are present even in non-allergic subjects, HDM-reactive T_{H} and T_{REG} cells were also isolated from asthmatic (N=6) and healthy subjects (N=6) without HDM allergy to uncover features that contribute to the lack of HDM allergy (FIG. 1A). HDM-reactive T_{H} cells (0.2-3% of all memory T_{H} cells) and T_{REG} cells (1-5% of all memory T_{REG} cells) were detected in all subject groups.

Single-cell transcriptomic analysis reveals heterogeneity in HDM allergen-reactive T cells—Analysis of the single-cell transcriptomes of >50,000 HDM-reactive T cells from allergic asthmatic subjects and relevant control groups (allergic non-asthmatic, asthmatic non-allergic to HDM and healthy subjects) revealed 7 distinct clusters present at varying frequency among subjects (data not shown). To understand the molecular properties unique to each cluster, multiple pair-wise single-cell differential gene expression analyses were performed. Several hundred genes (N=687) were especially highly expressed by each cluster, allowing classification into specific T_{H} subsets (FIGS. 2A-2B). Cells in cluster 1 were highly enriched for transcripts encoding canonical type 2 cytokine gene products (IL-5, IL-13, IL-4), the T_{H}2 master transcription factor GATA3, and surface receptors (IL1RL1 and IL17RB) for the T_{H}2-polarizing cytokines IL-33 and IL-25, indicating that this cluster represented bona fide T_{H}2 cells (FIGS. 2A-2B). A second cluster of cells was enriched for T_{H}1 phenotype- and function-related genes such as IFNG, CXCR3, and PRF1. A third cluster was enriched for T_{H}17 phenotype- and function-related genes such as IL17A, IL17F, CCR6, IL22. Three other clusters, although not associated with bona-fide canonical T_{H} subset biology were enriched for genes linked with cell activation, ribosomal proteins, RNA translation, endocytosis, and membrane trafficking.

A novel HDM-reactive T_{H} subset with IFN response signature increased in non-allergic subjects—The third largest cluster of HDM-reactive T cells was enriched for IFN response genes (IFI6, MX1, ISG20, OAS1, IFIT1, IFI44L) (Lee A J, et al., Front Immunol. 2018; 9:2061. Epub 2018 Sep. 27) indicating that they represent a previously uncharacterized T_{H} subset, which is termed as the T_{H} subset expressing the interferon response signature (T_{H}IFNR) (FIGS. 2A-2B). Gene set enrichment analysis (GSEA) confirmed enrichment of IFN response genes (Subramanian A, et al., Proc Natl Acad Sci USA. 2005; 102(43):15545-50. Epub 2005 Oct. 4) in this cluster (FIGS. 2A-2B). It was then determined if the proportions of any of the HDM-reactive subsets varied between subjects with or without HDM allergy or asthma (FIG. 3A). As expected, the T_{H}2 cluster was present only in the HDM-allergic groups consistent with the central role of T_{H}2 cells and type 2 cytokines in IgE class switching and allergic asthma pathogenesis (Georas S N, et al., Eur Respir J. 2005; 26(6):1119-37. Epub 2005 Dec. 2; Oettgen H C, et al., J Clin Invest. 1999; 104(7):829-35. Epub 1999 Oct. 8). On the other hand, the T_{H}1 cluster, though observed in all subject groups, was present at greater proportions in subjects without HDM allergy, consistent with the reciprocal role of T_{H}1 cells in dampening T_{H}2 differentiation. Most interestingly, in subjects without HDM allergy, both asthmatic and healthy control, displayed a substantially broader T_{H} response to HDM but failed to generate T_{H}2 cells and on the contrary, a large majority of HDM-reactive T_{H} cells were associated with the IFN response signature. This negative association with HDM-allergy led to the hypothesis that T_{H}IFNR cells may play a role in dampening T_{H}2 response to allergens and thus reduce allergic airway inflammation.

T_{H}IFNR cells express TRAIL that inhibits activation of T cells.

To determine the molecular properties of HDM-reactive T_{H}IFNR cells, the transcripts that are specifically enriched in this subset were examined. It was found that T_{H}IFNR cells expressed the highest levels of CXCL10 (FIGS. 3C-3D). CXCL10 encodes for a chemokine that recruits T_{H} cells expressing the receptor CXCR3, essentially T_{H}1 cells (Gauthier M, et al., JCI Insight. 2017; 2(13). Epub 2017 Jul. 7). T_{H}1 cells produce IFN-γ which is known to inhibit T_{H}2 differentiation as well as activation (Huber J P, et al., J Immunol. 2010; 185(2):813-7. Epub 2010 Jun. 18; Huber J P, et al., J Immunol. 2014; 192(12):5687-94. Epub 2014 May 13). Therefore, activation of T_{H}IFNR cells is likely to induce the recruitment of T_{H}1 cells, which in turn dampen T_{H}2 responses. T_{H}IFNR cells also expressed high levels of TNFSF10, which encodes Tumor necrosis factor-Related Apoptosis-Inducing Ligand (TRAIL) (FIGS. 3C-3D). Membrane bound TRAIL have been shown to induce apoptosis of cells expressing its receptor (TRAIL-R) (Groom J R, et al., Immunity. 2012; 37(6):1091-103. Epub 2012 Nov. 6; Peteranderl C, et al., Front Immunol. 2017; 8:313. Epub 2017 Apr. 7). Although TRAIL/TRAIL-receptors is a complex system, it has recently been shown to have the capacity to dampen TCR signaling by inhibiting the phosphorylation of downstream kinases (Lehnert C, et al., J Immunol. 2014; 193(8):4021-31. Epub 2014 Sep. 14; Chyuan I T, et al., Mucosal Immunol. 2019; 12(4):980-9. Epub 2019 May 12; Chyuan I T, et al., Cell Mol Immunol. 2018; 15(9):846-57. Epub 2017 Apr. 11). It was confirmed that following TCR stimulation TRAIL was expressed by population of T_{H} cells, which is likely to be enriched for the T_{H}IFNR subset (FIG. 3E). Given that activated T_{H} cells express TRAIL-R (Roberts A I, et al., Immunol Res. 2003; 28(3):285-93. Epub 2004 Jan. 10; Zhang X R, et al., Cell Death Differ. 2003; 10(2):203-10. Epub 2003 Apr. 18), TRAIL produced by T_{H}IFNR cells may play an important role in blocking T_{H} cell responses in vivo. TRAIL's function was experimentally tested and it was found that recombinant TRAIL inhibited TCR-dependent activation of T_{H} cells ex vivo, as measured by the surface expression of the activation markers (Bacher P, et al., J Immunol. 2013; 190(8):3967-76. Epub 2013 Mar. 13) (FIG. 3E). Although TRAIL's role has been ambiguous with conflicting studies, it has been shown to reduce inflammation in disease models including allergic inflammation (Benedict C A, et al., J Exp Med. 2012; 209(11):1903-6. Epub 2012 Oct. 24; Chyuan I T, et al., Cell Mol Immunol. 2018; 15(9):846-57. Epub 2017 Apr. 11; Tisato V, et al., Cell Mol Life Sci. 2016; 73(10):2017-27. Epub 2016 Feb. 26; Zauli G, et al., Diabetes. 2010; 59(5):1261-5. Epub 2010 Feb. 27; Faustino L, et al., Mucosal Immunol. 2014; 7(5):1199-208. Epub 2014 Feb. 27). Therefore, without wishing to be bound by the theory, it was hypothesized that these TRAIL-expressing HDM-reactive T cells could play an important role in dampening T_{H}2 inflammation in allergy and asthma.

HDM-Reactive T_{REG} Cells with IFN-Response are Increased in Non-Allergic Patients.

It was also investigated whether HDM-reactive T_{REG} cells differed between HDM allergic and non-allergic subjects. It was confirmed that the proportion of HDM-reactive T_{REG} cells was not related to HDM allergic status. To determine whether specific subsets of HDM-reactive T_{REG} cells varied with disease state, performed was a single-cell transcriptomic analysis of ˜10,000 HDM-reactive T_{REG} cells across the 4 subject groups (FIGS. 4A and 4D-4E). Interestingly, the proportion of cells for one cluster was greater in non-allergic asthmatics compared with allergic asthmatics, suggesting a preferential expansion of this subset in asthmatic subjects without HDM allergy (FIGS. 4A and 4D-4E). GSEA analysis of transcripts enriched in this cluster (N=248) revealed significant enrichment of IFN response genes (FIGS. 4A and 4D-4E). The features of this T_{REG} cluster were similar to those of the T_{H}IFNR cluster, which was also present at higher proportions in non-allergic subjects; for example, IFN responsive T_{REG} cells (T_{REG}IFNR) also expressed higher levels of transcripts encoding for TRAIL (FIGS. 4A and 4D-4E).

T_{H}IFNR cells are present in lungs/airways from mouse models of asthma—A recent single-cell transcriptomic study identified T_{H} cells expressing the IFN response signature in the lung tissue of mice sensitized and challenged with HDM (Tibbitt et al., Immunity. 2019; 51(1):169-84 e5. Epub 2019 Jun. 25). This finding implies that T_{H}IFNR cells can be generated and sustained in vivo, and that HDM sensitization of mice may be an appropriate system to test the role of these cells in allergic inflammation. Further studies, as disclosed herein, are required to determine T_{H}IFNR cells function in vivo and understand the molecular mechanisms and signals that drive their differentiation and persistence.

T_{H}IFNR cells are present in lungs/airways of asthmatics patients and control subjects—To corroborate the findings in mouse models of asthma, examined is the single-cell RNA-seq data generated from CD4⁺ T cells present in bronchoalveolar lavage specimens from 26 patients with mild and severe asthma (FIG. 11). Although tissue-resident memory T cells (T_{RM} cells) were the major CD4 T cells subset in the airway, it was found that a small fraction of cells (3%) expressed the IFN response signature genes i.e., were T_{H}IFNR cells (FIG. 11). Importantly, these T_{H}IFNR cells were enriched for the expression of tissue-residency gene, suggesting that they are tissue-resident cells and not likely to re-circulate in the blood. Although, T_{RM} cells have only recently been identified, their importance in asthma pathogenesis and protection against viral infection is well established (Rahimi R A, et al., J Exp Med. 2020; 217(9). Epub 2020 Jun. 25; Faustino L, et al., Mucosal Immunol. 2014; 7(5):1199-208. Epub 2014 Feb. 27; Beura L K, et al., Nat Immunol. 2018; 19(2):173-82. Epub 2018 Jan. 10; Hondowicz B D, et al., Immunity. 2016; 44(1):155-66. Epub 2016 Jan. 12; Clarke J, et al., J Exp Med. 2019; 216(9):2128-49. Epub 2019 Jun. 23). To further explore, if tissue-resident T_{H}IFNR cells are present in normal lung tissue (non-asthmatics), published single-cell datasets were explored and it was found that CD4⁺ T cells in human lungs expressed interferon-response signature genes and TRAIL, which indicated that T_{H}IFNR subset is also present in the human lung tissue (data not shown). Together, without wishing to be bound by the theory, these findings suggest that the tissue-resident (airway) T_{H}IFNR cells, by expressing TRAIL like in their circulating counterparts, play an important role in dampening allergic inflammation. That this population is present in at higher levels in healthy patients, as compared to the levels seen in asthmatic/allergic patients/virally infected (see below), is supportive that these cells play a protective role

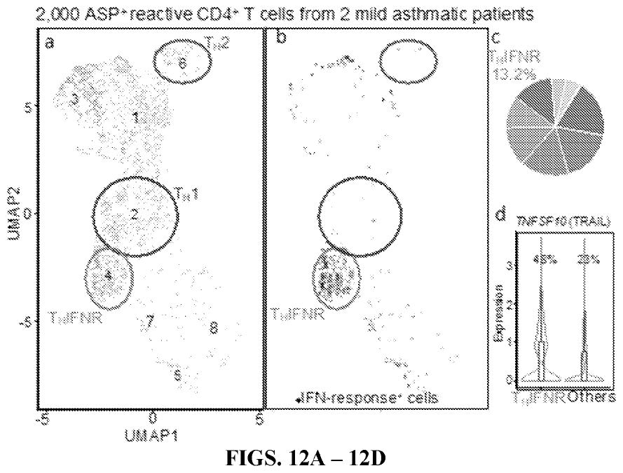

T_{H}IFNR cells are generated in response to other allergens—In order to see if T_{H}IFNR cells were specific to HDM or if they were also reactive to other allergens, (in other words, whether T_{H}IFNR cells were generated as a stable CD4⁺ memory subset in response to other allergens), CD4 T cells reactive to another important aero-allergen, Aspergillus fumigatus (ASP) were analyzed. ARTE assay (Seumois G, et al., Sci Immunol. 2020; 5(48). Epub 2020 Jun. 14; Bacher P, et al., Cell. 2016; 167(4):1067-78 e16. Epub 2016 Oct. 25; Frentsch M, et al., Nat Med. 2005; 11(10):1118-24. Epub 2005 Sep. 28) was performed using peptide pools specific to ASP and responding memory CD4⁺ T cells were isolated for single-cell RNA-seq analysis. It was found that 13.2% of ASP-reactive CD4⁺ memory T cells expressed IFN response signature i.e., were T_{H}IFNR cells (FIG. 12). This finding suggests that T_{H}IFNR cells can be generated against other common allergens.

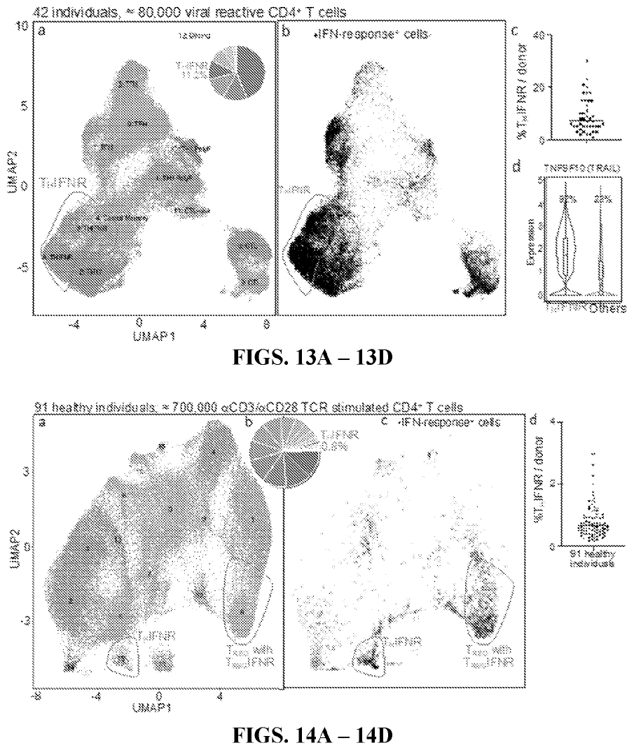

T_{H}IFNR cells are generated in response to viral infections—In order to see whether T_{H}IFNR cells were also generated in response to viral infections, a large-scale single-cell transcriptomic data of viral-reactive CD4⁺ T cells generated (Meckiff B J, et al) were used. This dataset included SARS-CoV-2-reactive CD4⁺ T cells from patients with COVID-19 illness, and influenza, parainfluenza- and metapneumo-virus-reactive CD4⁺ memory T cells from healthy subjects. Clustering analysis of virus-reactive CD4⁺ T cells showed multiple clusters of cells, including T_{H}1, T_{H}17, T_{CM}, T_{FH}, and cytotoxic-CD4 T cells (FIG. 13). Most importantly, a distinct cluster of cells were enriched in IFN response signature genes in both COVID-19 patients and healthy subjects. The viral-reactive T_{H}IFNR subset comprised nearly 11% of all viral-reactive cells and was not specific to any virus or COVID-19 patients (Meckiff B J, et al). This finding suggests that T_{H}IFNR cells may have a broader role in diverse immune responses (allergic and viral). Given the importance of viral infections in asthma attacks, it is tempting to speculate if viral-specific T_{H}IFNR cells play a role in dampening airway inflammation.

T_{H}IFNR cells are observed as a stable subset in healthy subjects—If T_{H}IFNR cells are generated in response to allergens and viruses and are a stable memory population then it would be expected to see these cells as a ubiquitous population in healthy subjects. Here, a single-cell RNA-seq analysis of 91 healthy subjects enrolled in the DICE project was performed (dice-database.org) (Schmiedel B J, et al., Cell. 2018; 175(6):1701-15 e16. Epub 2018 Nov. 20). In this study, memory CD4⁺ T cells were stimulated with anti-CD3 and anti-CD28 for 6 hours and single-cell RNA-seq performed on the activated cells. In total >1 million single-cell transcriptomes were analyzed, comprising of ˜10,000 single cells per subject. It was found that nearly 1% of all cells expressed IFN response signature and had all features indicative of T_{H}IFNR cells (FIG. 14). This population of cells was consistently seen in all individuals (FIG. 14). This finding from a large-scale study suggests that T_{H}IFNR cells are indeed a stable and ubiquitous T_{H} subset that has eluded discovery by immunologists for decades. The wide-use of single-cell unbiased transcriptomic analysis in immunology helped its discovery.

Summary—Extensive preliminary data (FIGS. 1-14), obtained from the multiple single-cell RNA-seq studies performed in various contexts provide a compelling argument for investigating the role of a new subset of T_{H} cells. More specifically, without wishing to be bound by the theory, in the context of allergy and asthma, it was hypothesized that this population may play an “anti-T_{H}2” role and curb type 2 inflammation. A better understanding of the function of those cells as well as their development could bring novel therapeutic opportunities to cure or control allergies and asthma.

More details about the experimental methods and assays are provided below.

The single-cell transcriptomic analysis revealed the existence of a novel CD4⁺ T cell subset—T_{H}IFNR cells—in the blood and airways/lungs of healthy and asthmatic individuals. Furthermore, it was found that HDM allergen-specific T_{H}IFNR are highly enriched in subjects who neither generate HDM-specific T_{H}2 cells nor develop HDM allergy, suggesting they may antagonize the development of T_{H}2 responses to allergen and impact asthma outcomes. It was shown that expression of TNFSF10 was enriched in T_{H}IFNR cells, and its product, TRAIL, dampened activation of T_{H} cells in vitro. These findings suggest that the T_{H}IFNR may dampen allergic responses in asthma. Here, to test this hypothesis, mouse models of HDM-induced allergic airway inflammation (asthma) are employed. As explained in more detail below in Point 1 below, the characteristics and functional role of T_{H}IFNR cells are defined in vivo with special emphasis on the role of TRAIL. In Point 2 as detailed below, unbiased enhancer profiling of T_{H}IFNR cells are defined what transcription factors play a role in the development and maintenance of these cells.

1: The Functional Role of T_{H}IFNR Cells In Vivo

Systematically addressed below is how T_{H}IFNR cells modulate allergic airway inflammation in mouse models of asthma. More specifically, the following questions are answered.

(i) What are the functional characteristics of T_{H}IFNR cells generated in vivo? What is their association with the degree of allergic inflammation observed in different models of allergic asthma?

(ii) Do T_{H}IFNR cells inhibit allergic airway inflammation and the development of T_{H}2 cells in vivo?

(iii) Is TRAIL expression in T_{H}IFNR cells required for inhibiting allergic airway inflammation?

1A. The functional characteristics of T_{H}IFNR cells in asthma models using ISRE reporter mice.

In mouse models of asthma, the number of T_{H}IFNR cells in the airways/lungs will be negatively associated with that of T_{H}2 cells and the degree of allergic airway inflammation.

The function of immune cells in vivo can be best studied in model organisms where manipulation of cell function can be done without significant risks. Murine models of allergic airway inflammation are widely used to define mechanisms of the immunopathology of allergic asthma (Stevenson C S, et al., Pharmacol Ther. 2011; 130(2):93-105. Epub 2010 Nov. 16; Aun M V, et al., J Asthma Allergy. 2017; 10:293-301. Epub 2017 Nov. 22). In these models the hallmarks of allergic asthma and adaptive immune responses, including allergen-specific IgE and T_{H}2 responses are induced by sensitization and challenge with allergens (HDM, fungal allergens) or antigens with adjuvants (e.g. ovalbumin+alum). One type of model that has been shown to be clinically relevant, is a model with HDM sensitization (intranasal) and challenge (Debeuf N, et al., Curr Protoc Mouse Biol. 2016; 6(2):169-84. Epub 2016 Jun. 2). This model and multiple variations of it have been widely used to study experimental allergic asthma, and hence, it is used as the standard ‘asthma-inflammation model’ herein (Debeuf N, et al., Curr Protoc Mouse Biol. 2016; 6(2):169-84. Epub 2016 Jun. 2). Modifying the adjuvants or addition of TLR agonists (HDM+LPS) during the sensitization phase has been shown to result in protection or reduction in HDM-induced allergic airway inflammation. This HDM+LPS model (‘asthma-protection model’) is used as a model in which HDM-induced airway inflammation is relatively less compared to standard ‘asthma-inflammation model’ (Bachus H, et al., Immunity. 2019; 50(1):225-40 e4. Epub 2019 Jan. 13; Schuijs M J, et al., Science. 2015; 349(6252):1106-10. Epub 2015 Sep. 5).

Are T_{H}IFNR cells detected in mouse asthma models? It has been recently shown that T_{H}IFNR cells are generated in the standard HDM-induced asthma mouse model (Tibbitt C A, et al., Immunity. 2019; 51(1):169-84 e5. Epub 2019 Jun. 25). It was shown by single-cell RNA-seq analysis of 764 CD4⁺ T cells in BAL specimens (FIGS. 1C, 1D, 2B and 2D of Tibbitt et al. 2019). 6 clusters of cells were identified, which included the canonical T_{H}2, T_{H}1, and T_{H}17 subsets. The cluster 6 showed strong enrichment in IFN response genes such as MX1, ISG20, and IFIT3, reminiscent of the T_{H}IFNR cells discovered in the single-cell studies in humans as disclosed herein. T_{H}IFNR cells represent ≈3% of all CD4⁺ T cells compared to 25% for T_{H}2 cells. Importantly, it was showed that by treating mice with a neutralizing antibody directed against type I IFN receptor (IFNAR1), the expression of the IFN-induced genes by CD4 T cells was significantly reduced, confirming the role of type I IFNs in the generation of T_{H}IFNR cells. This evidence supports the use of HDM-induced asthma models for studying the function of T_{H}IFNR cells.

Why IFN reporter mice are required to characterize T_{H}IFNR cells in vivo? In humans and mice, the IFN family is divided into type I (IFN-α/β), II (IFN-γ), and III (IFN-λ) (McNab F, et al., Nat Rev Immunol. 2015; 15(2):87-103. Epub 2015 Jan. 24; Lee A J, et al., Front Immunol. 2018; 9:2061. Epub 2018 Sep. 27). Their receptors are INFAR1/2, IFNGR1/2, IL28RA/IL10R, respectively. With the exception of type III IFNs which are restricted to neutrophils and epithelial cells, IFN receptors are expressed ubiquitously on all nucleated cells, including T cells. T_{H}IFNR cells express interferon-response genes. To date there are no surface or intracellular markers to reliably detect T_{H}IFNR cells using standard FACS analysis for characterization or isolation for downstream studies. Since the expression of these genes is the results of signaling cascade following IFN engagement with their cognitive receptors, T_{H}IFNR cells can be detected using the following IFN-stimulated response element (ISRE) reporter mice:

(i) Mx1 GFP reporter mice (B6.Cg-Mx1^{tm1.1Agsa}/J) (Uccellini M B, et al., Cell Rep. 2018; 25(10):2784-96 e3. Epub 2018 Dec. 6): In this C57BL/6 mouse strain GFP reporter gene is knocked into the endogenous Mx1 locus and expressed under the control of Mx1 promoter. Mx1 encodes a protein that participates in the cellular antiviral response. Only type I and III IFNs induce the expression of Mx1, not type II IFNs. Notably, T cells neither express type III IFNs nor their receptors making this Mx1, a type I IFN specific reporter for T cells. The inclusion of GFP does not have any functional impact on this strain (Staeheli P, et al., Mol Cell Biol. 1988; 8(10):4518-23. Epub 1988 Oct. 1). A breeding colony of MX1 GFP mice (Jackson Lab) are used for these experiments.

(ii) Irgm1-DsRed reporter mice (Stifter S A, et al., Cell Rep. 2019; 29(11):3539-50 e4. Epub 2019 Dec. 12): This reporter strain, a gift from Dr. Feng (University of Sydney), has a DsRed gene knocked into the translational start site of the IFN-g-related GTPase ml (Irgm1) gene. This gene is induced by signaling of all 3 IFNs types and thus these mice report expression of all IFNs.

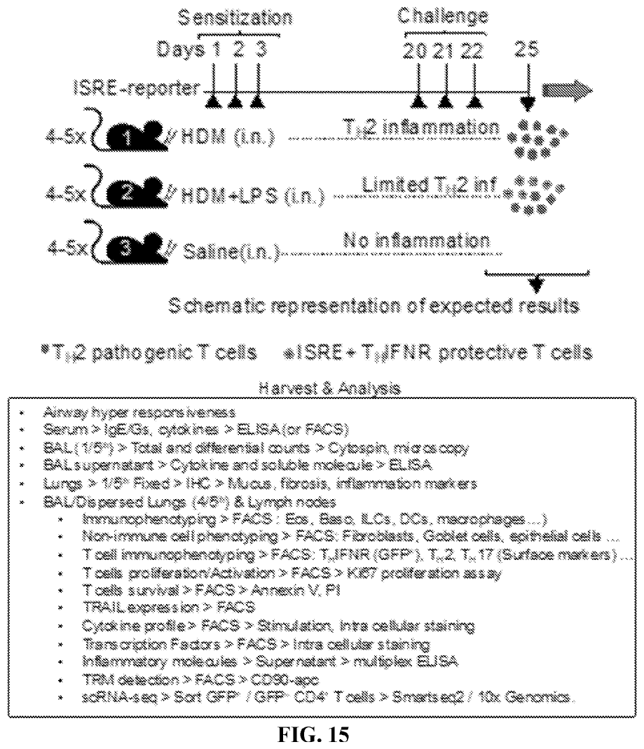

Methods (FIG. 15)—Asthma model: IFN reporter mice are ensitized and challenged with either HDM (‘asthma-inflammation model’) or HDM+LPS (‘asthma-protection model’) and compared with saline control mice. Additional asthma models as below are considered and used.

Readouts: (i) Quantify T_{H}IFNR and T_{H}2 cells in airway/lungs: T_{H}IFNR cells are quantified based on expression of GFP/DsRed in the CD4⁺ T cells obtained from BAL and dispersed lung tissue samples (as described (Debeuf N, et al., Curr Protoc Mouse Biol. 2016; 6(2):169-84. Epub 2016 Jun. 2; Clarke J, et al., J Exp Med. 2019; 216(9):2128-49. Epub 2019 Jun. 23; Vijayanand P, et al., Immunity. 2012; 36(2):175-87. Epub 2012 Feb. 14)). Five minutes before harvesting organs, mice are treated intraperitoneally with anti-CD90.1-APC antibodies in order to be distinguish circulating immune cells from lung-resident T cells. Airways T_{H}2 cells, eosinophils and basophils are enumerated and their ability to produce type 2 cytokines (IL-4, IL-13, and IL-5) are tested ex vivo. (ii) Phenotype of T_{H}INFR cells: resting and stimulated airway/lung cells are stained with a cocktail of antibodies to determine their (a) proliferation status (Ki67), (b) degree of apoptosis (Annexin V/PI), (c) TRAIL expression, (d) cytokine profile (IL-4, -13, -5, -17, -21, -2, -12, IFN-γ, TNF, CXCL10), (e) transcription factor expression (FOXP3, GATA3, RORG, T-bet, . . . ), and (f) tissue-residency status (CD69⁺ and CD90.1 negative cells). (iii) Transcriptomic profile of T_{H}IFNR cells: FACS-sorted T_{H}IFNR cells (GFP⁺) and GFP-ve CD4⁺ T cells (non-T_{H}IFNR cells) are processed for RNA-seq analysis using the Smart-seq2 platform (Picelli S, et al., Nat Methods. 2013; 10(11):1096-8. Epub 2013 Sep. 24) that is well optimized (Rosales S L, et al., Methods Mol Biol. 2018; 1799:275-302. Epub 2018 Jun. 30). The most differentially expressed transcripts in T_{H}IFNR cells that are encoding for effector molecules are validated at the protein level. (iv) Measure of airway responsiveness: to increasing dosage of β-methacholine (2.5-20 mg/ml; Sigma-Aldrich) using a barometric plethysmograph (Flexivent) (Debeuf N, et al., Curr Protoc Mouse Biol. 2016; 6(2):169-84. Epub 2016 Jun. 2; Palipane M, et al., Microb Pathog. 2019; 127:212-9. Epub 2018 Dec. 12). (v) Measure of allergic airway inflammation: Differential cell count is performed on BAL cells to assess degree of type 2 inflammation. A fragment of lung is perfused and immersed in 4% paraformaldehyde fixation solution overnight, stored in OCT resin for standard immunohistochemistry staining to assess degree of airway inflammation and remodeling (Miller M, et al., J Immunol. 2014; 192(8):3475-87. Epub 2014 Mar. 14).

Extensive characterization of T_{H}IFNR cells is generated in the HDM-induced (‘asthma-inflammation model’) or HDM+LPS-induced (‘asthma-protection model’). It also is determined if increased numbers of T_{H}IFNR cells in the airways is associated with a reduction in T_{H}2 cells and type 2 airway inflammation. If T_{H}IFNR cells are playing an important role in reducing type 2 inflammation in the ‘asthma-protection model’, then it is expected to see higher ratio of T_{H}IFNR cells to T_{H}2 cells in this model when compared to the ‘asthma-inflammation model’ (FIG. 15).

1B. The Function of T_{H}IFNR Cells in Allergic Airway Inflammation (Asthma Models).

Without being bound by theory, Applicant submits that failure to generate T_{H}IFNR cells (mice with T cell-specific deletion of type I IFN receptor) results in worsening of allergic airway inflammation in mouse models of asthma.

Without wishing to be bound by the theory, T_{H}IFNR cells develop following TCR engagement with co-stimulatory signals and type I IFNs with or without co-signaling from type II IFNs. Therefore, blocking type I IFNs signaling pathways in HDM-asthma model is likely to impede the development of T_{H}IFNR cells (Tibbitt C A, et al), and thus permits assessing its impact on allergic airway inflammation. First, type I IFNs pathway(s) are pharmacologically blocked; second, genetically-modified mice that cannot activate IFN pathways (global IFNAR1 knockout) are used; third, conditional IFNAR1 mice are used to block type I IFN signaling specifically in T cells. Finally, adoptive transfer studies is performed to test the anti-T_{H}2 protective role of T_{H}IFNR cells.

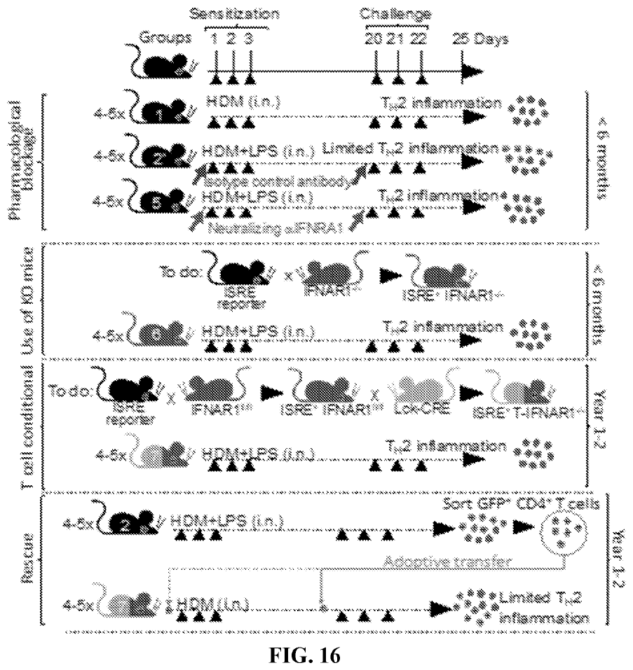

Methods (FIG. 16)—The following are utilized: (i) HDM (‘asthma-inflammation model’) and (ii) HDM+LPS (‘asthma-protection model’) to assess impact of blocking type 1 IFNs on allergic inflammation.

Loss-of-function studies—Complete blockage/absence of type I IFNs signaling—One of the 2 subunits of the type I IFNs receptor, IFNAR1 is blocked in 2 ways, one by using neutralizing antibodies and second by using global knock-out mice (Muller U, et al., Science. 1994; 264(5167):1918-21. Epub 1994 Jun. 24; Park C, et al., Immunity. 2000; 13(6):795-804. Epub 2001 Feb. 13). (i) Pharmacological inhibition: IFN-reporter mice are treated with an anti-mouse IFNAR1 neutralizing antibody (BioCell, clone: MAR1-5a3) before sensitization (intraperitoneally 500 μg/mouse) and during challenges (Tibbitt C A, et al., 5; Uccellini M B, et al., Cell Rep. 2018; 25(10):2784-96 e3. Epub 2018 Dec. 6). As controls, anti-mouse IgG1 isotype is used. (ii) IFNAR1^{-/-} and STAT2^{-/-} mice: IFN-reporter mice are crossed with both knock-out mice strains: IFNAR1^{-/-} and STAT2^{-/-} (STAT2 being a canonical factor involved in the type I IFNs signaling). Both strains are tested in both asthma models. Those strains are available to one of skill in the art, while conditional mice are being generated (FIG. 16). T cell-specific absence of type I IFNs signaling—In order to selectively block the type I IFNs signaling pathways only in T cell lineage, T cell conditional knock-out mice for IFNAR1 are generated. Ifnar1^{fl/fl} (Jackson lab) are crossed with Lck-Cre mouse strain to produce mice that specifically lack IFNAR1 in T cells. Without being bound by theory, T_{H}IFNR cells play an important role in dampening T_{H}2 inflammation, by blocking IFNAR1 receptor signaling (complete blockage); fewer T_{H}IFNR cells (GFP⁺ cells) and an increase in T_{H}2 cells and allergic airway inflammation is observed in both models.

Gain-of-function studies—The role of T_{H}IFNR cells in impeding development of T_{H}2 cells and allergic inflammation in vivo, can be confirmed using methods known in the art, e.g., adoptive transfer of T_{H}IFNR cells (GFP⁺ cells from reporter mice, as described herein) into wild type and IFNAR1^{fl/fl}×Lck-CRE is performed. T_{H}IFNR is isolated from the HDM+LPS-induced asthma model and between 10⁵ to 10⁶ T_{H}IFNR cells is transferred intravenously into recipient mice before sensitization or challenge. Both models (HDM+LPS and HDM alone) are tested, and the impact of transferring T_{H}IFNR cells on allergic airway inflammation is assessed as described herein. Appropriate controls will be performed accordingly (e.g., transfer of GFP-negative cells). Without being bound by theory, the transfer of T_{H}IFNR cells in mice with IFNAR1-deficient T cells antagonize T_{H}2 inflammation and reduce allergic airway inflammation (FIG. 16).

1C. The Role of TRAIL in the Function of T_{H}IFNR Cells in Allergic Airway Inflammation.

T_{H}IFNR cells deficient in TRAIL fail to suppress allergic airway inflammation.

In humans TRAIL has been associated with 5 receptors: TRAIL-R1/DR4 and -R2/DR5 contain a ‘death domain’ and lead to apoptosis. Although TRAIL/TRAIL-Rs is a complex system, it has been shown that both, surface-bound and soluble TRAIL, have the capacity to dampen TCR signaling by inhibiting the phosphorylation of downstream kinases (Lehnert C, et al., J Immunol. 2014; 193(8):4021-31. Epub 2014 Sep. 14; Chyuan I T, et al., Mucosal Immunol. 2019; 12(4):980-9. Epub 2019 May 12; Roberts A I, et al., Immunol Res. 2003; 28(3):285-93. Epub 2004 Jan. 10). Given that activated T_{H} cells express TRAIL-R (Roberts A I, et al., Immunol Res. 2003; 28(3):285-93. Epub 2004 Jan. 10; Zhang X R, et al., Cell Death Differ. 2003; 10(2):203-10. Epub 2003 Apr. 18), TRAIL produced by T_{H}IFNR cells may play an important role in blocking T_{H} cell responses in vivo. It was reported that HDM-reactive T_{H}IFNR cells expressed the highest levels of transcripts encoding for TRAIL. Consistent with published reports, TRAIL was reported to suppress TCR-dependent activation of human T cells ex vivo, as measured by the surface expression of the activation markers CD154, CD69, and CD137 (4-1BB) (FIG. 3E). Studies in mouse models of inflammatory diseases have shown that blocking TRAIL worsens inflammation, suggesting a potential anti-inflammatory role in vivo. Therefore, without wishing to be bound by the theory, TRAIL expressed on T_{H}IFNR cells is believed to play an important anti-inflammatory role (Benedict C A, et al., J Exp Med. 2012; 209(11):1903-6. Epub 2012 Oct. 24; Chyuan I T, et al., Cell Mol Immunol. 2018; 15(9):846-57. Epub 2017 Apr. 11; Tisato V, et al., Cell Mol Life Sci. 2016; 73(10):2017-27. Epub 2016 Feb. 26; Zauli G, et al., Diabetes. 2010; 59(5):1261-5. Epub 2010 Feb. 27; Faustino L, et al., Mucosal Immunol. 2014; 7(5):1199-208. Epub 2014 Feb. 27).

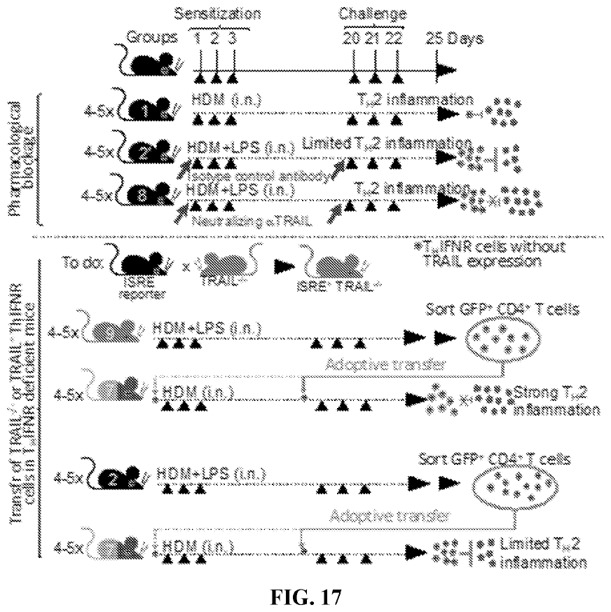

Methods (FIG. 17)—An anti-mTRAIL blocking antibody (clone N2B2) is used to block TRAIL/TRAIL-R engagement pathways. This Ab is injected (200 μg/mouse) intraperitoneally in ISRE-reporter mice before sensitization and/or challenge with HDM (HDM+LPS) and the response of this treatment is evaluated on T_{H}2 cell numbers and allergic airway inflammation.

A complete knockout TRAIL^{-/-} mice strain is used. This strain is crossed with the ISRE-reporter mice and TRAIL^{-/-}×ISRE-reporter mice are sensitized/challenged with HDM-LPS to isolate TRAIL-deficient T_{H}IFNR cells (GFP⁺) in vivo. As described in the gain-of-function studies, TRAIL-sufficient and TRAIL-deficient T_{H}IFNR cells is adoptively transferred into wild type and IFNAR1^{fl/fl}×Lck-CRE (mice with IFNRA1-deficient T cells) prior to sensitization and challenge with HDM (HDM-LPS) (FIG. 17). TRAIL-deficient T_{H}IFNR cells are then analyzed for effectiveness in impeding allergic airway inflammation and the generation of airway T_{H}2 cells. As described herein, these adoptive transfer studies use DER-1 transgenic mice.

Systemic treatment with TRAIL neutralizing antibodies should block the function of T_{H}IFNR role and promote a stronger inflammation. Where it is found that TRAIL-deficiency has no effect on the function of T_{H}IFNR cells in vivo, then the role of other candidate molecules is investigated. For example, the role of other candidate molecules, such as CXCL10 or targets are likely to emerge from the characterization studies (RNA-seq) described herein, and are tested on the function of T_{H}IFNR cells in vivo.

For animal studies, the number of mice per group was calculated to achieve 95% power for detecting statistical differences. Each experiment uses at least 4-6 mice/group. To ensure allocation, treatment and handling of mice is equal, animals within each experimental group are randomized and groups subjected to blinded analysis. Mean response among groups are compared, and data reported as mean±SD. Sex as a biological variable—Studies have shown difference in response to allergen challenge experiments between males and females. First data sets are generated only with female and are confirmed with another set of experiment using males. Differences or trends between males and females are investigated and any differences are evaluated with experiments and analysis.

2—Transcription Factors Driving the Development and Maintenance of Human T_{H}IFNR Cells

Transcription factors (TFs) drive the differentiation and function of T_{H}IFNR cells.

Since the discovery of CD4⁺ T cell subsets, a major quest in T cell biology has been to understand the signals that control the differentiation of the canonical memory subsets. For example, it has been shown that naive CD4⁺ T cells differentiate into T_{H}1 cells following co-activation signals including IL-12 and IFN-γ (type II IFN) that result in the activation of the TFs STAT4 and T-bet (Zhu J, et al., Annu Rev Immunol. 2010; 28:445-89. Epub 2010 Mar. 3). However, the signals that induce the differentiation of a stable population of T_{H}IFNR cells, and the influence of type I IFNs on the commitment of naive T cells is less clear. Type I IFNs activate a canonical pathway with STAT2, STAT1 and Interferon response factor 9 (IRF9) to form the interferon-sensitive gene factor-3 (ISGF3) that subsequently relocates in nucleus and binds to IFN-stimulated response elements (ISREs) to induce transcription of target genes (Ivashkiv L B, et al., Nat Rev Immunol. 2014; 14(1):36-49. Epub 2013 Dec. 24). Though not directly driving proliferation, type I IFNs have been showed to block apoptosis following antigen stimulation in vitro and to promote the development of human central memory-like CD4⁺ T cells with elevated IL-2 expression (Huber J P, et al., J Immunol. 2010; 185(2):813-7. Epub 2010 Jun. 18; Huber J P, et al., J Immunol. 2014; 192(12):5687-94. Epub 2014 May 13). Therefore, type I IFNs can act as factors promoting long-memory cell development by selectively upregulating IL-2 expression at the expense of pro-inflammatory cytokine secretion. However, to date, there is no evidence to show that type I IFNs by themselves can induce reorganization of chromatin in naive T cells to commit their differentiation to a stable CD4⁺ memory T cell subset i.e., T_{H}IFNR cells. Furthermore, it is not clear whether other TFs cooperate with IFN signaling factors to imprint a stable memory phenotype, as T_{H}IFNR cells are a ubiquitous population of CD4⁺ T cells even in healthy subjects.

TFs identified via single-cell RNA-seq analysis of HDM-reactive T cells. Having defined T_{H}IFNR cells, TFs that are highly expressed in the single-cell RNA-seq data was investigated and TF motif enrichment analysis was performed for the promoters of genes enriched for expression in T_{H}IFNR cells. Although several transcription factors (TF) belonging to the type I IFNs signaling pathway (e.g., IFN regulatory factors (IRFs), STATs, SOCS) were found, none of these TFs have been shown to direct the differentiation of naive T cells into T_{H}IFNR cells upon TCR engagement. Therefore, it is highly conceivable that other novel TFs or combination of already known signaling factors play an important role in the differentiation and maintenance of T_{H}IFNR cells.

Value of enhancer profiling and TF footprint analysis: Upstream regulators, such as TFs, involved in differentiation cannot always be identified by simple RNA expression profiling of fully differentiated cells. Many such TFs are only expressed transiently at early developmental stages, such as during priming—which may be challenging to uncover at a later time point. However, lineage-defining pioneering TFs leave a footprint of their activity on enhancers by displacing nucleosomes, creating transposase-accessible sites i.e., enhancer regions that can be accessed by other signaling-induced TFs like STATs to maintain cell fates. Thus, analysis of enhancer profiles in fully differentiated T_{H}IFNR cells is likely to provide insights into the key TFs that instruct differentiation of T_{H}IFNR cell fate in the absence of exogenous IFN signals. The Examples as disclosed herein take advantage of genome-wide epigenetic profiling to characterize specific cell populations (Seumois G, et al., Nature immunology. 2014; 15(8):777-88. Epub 2014 Jul. 7; Engel I, et al., Nature immunology. 2016; 17(6):728-39. Epub 2016 Apr. 19). Here, T_{H}IFNR-specific active enhancers are identified to uncover TFs that play a role in the generation and maintenance of T_{H}IFNR cells. Single-cell (sc)ATAC-seq analysis was chosen to identify enhancers because these assays can be performed with small cell numbers (˜10³-10⁴ cells) (Seumois G, et al., Nature immunology. 2014; 15(8):777-88. Epub 2014 Jul. 7; Dahl J A, et al., Nucleic Acids Res. 2008; 36(3):e15. Epub 2008 Jan. 19; Schmidl C, et al., Nat Methods. 2015; 12(10):963-5; Dahl J A, et al., Nat Protoc. 2008; 3(6):1032-45. Epub 2008 Jun. 10; Bravo Gonzalez-Blas C, et al., Nat Methods. 2019; 16(5):397-400. Epub 2019 Apr. 10; Jia G, et al., Nature communications. 2018; 9(1):4877. Epub 2018 Nov. 20; Mezger A, et al., Nat Commun. 2018; 9(1):3647. Epub 2018 Sep. 9; Rai V, et al., Mol Metab. 2020; 32:109-21. Epub 2020 Feb. 8) and it has a higher resolution. scATAC-seq assays reliably identify enhancers by revealing regions of chromatin accessibility, which correlate with nucleosome-depleted regions (Agarwal S, et al., J Allergy Clin Immunol. 1999; 103(6):990-9. Epub 1999 Jun. 9; Ansel K M, et al., Nat Immunol. 2004; 5(12):1251-9. Epub 2004 Nov. 2; Cruz-Guilloty F, et al., The Journal of experimental medicine. 2009; 206(1):51-9. Epub 2009 Jan. 14; Djuretic I M, et al., Nature immunology. 2007; 8(2):145-53. Epub 2007 Jan. 2; Pipkin M E, et al., Immunol Rev. 2010; 235(1):55-72. Epub 2010 Jun. 12; Solymar D C, et al., Immunity. 2002; 17(1):41-50. Epub 2002 Aug. 2), and serves as the primary assay in the Examples (Corces M R, et al., Nat Methods. 2017; 14(10):959-62. Epub 2017 Aug. 29).

(i) Human T_{H}IFNR cells: To identify enhancers specific to activated fully differentiated human T_{H}IFNR CD4⁺ cells, combined scATAC-seq and scRNA-seq assay are performed in HDM-reactive CD4⁺ T cells from up to 12 subjects without HDM allergy, in whom a substantial number of T_{H}IFNR cells was observed (FIG. 1). At least 10% of HDM-reactive CD4⁺ T cells to be T_{H}IFNR cells is likely. HDM-reactive CD4⁺ T cells are barcoded and pooled from each subject to obtain >10,000 cells. Then, using commercially available kits (Chromium single-cell multiome ATAC+gene expression, 10× Genomics), scRNA- and scATAC-seq are performed on the same cells. (ii) Murine T_{H}IFNR cells: To better understand the TFs involved in the various stages of differentiation of T_{H}IFNR cells, especially in compartments that are relatively inaccessible (draining lymph nodes) in humans, scATAC-seq are performed in T_{H}IFNR cells (GFP/DsRed⁺ cells) and non-T_{H}IFNR cells (GFP/DsRed-ve) generated in vivo from ISRE-reporter mice subjected to the HDM-induced-asthma model. T_{H}IFNR cells are isolated from BAL, lungs, lung draining lymph nodes and spleen at specific time-points: D6 (priming), and D19, 25 (pre- and post-challenge) over the course of allergic airway inflammation (as described in 1A).

Bioinformatic analysis: For single-cell studies of HDM-reactive human T_{H}IFNR cells, scRNA-seq profile are first used to discriminate T_{H}IFNR cells from other CD4⁺ T cells subsets. The enhancer landscape (chromatin accessibility) of T_{H}IFNR cells are compared with that of non-T_{H}IFNR cells to determine which enhancers are specific to human T_{H}IFNR cells. Similarly, the chromatin accessibility profile of murine T_{H}IFNR cells (GFP⁺) and non-T_{H}IFNR cells (GFP-ve) are compared to define enhancers that specific to murine T_{H}IFNR cells at different stages of differentiation (Day 6, 19, 25 analysis). These studies provide a list of T_{H}IFNR cell-specific enhancers that are species conserved and human-specific. Single-cell ATAC-seq data is analyzed to predict enrichment of TF binding sites at enhancers specific to T_{H}IFNR cells generated in both humans and mice. PageRank TF analysis (Chen R, et al., Immunity. 2014; 41(2):325-38. Epub 2014 Aug. 26) are employed, which is highly effective in predicting critical TFs involved in defining cellular fates. Correlation of enhancer and promoter DNA accessibility measured using scATAC-seq are used to link enhancers, and hence TFs, to their putative target genes. Single-cell trajectory analysis are explored to define cells in an intermediately differentiated (Bergen V, et al., Nat Biotechnol. 2020. Epub 2020 Aug. 5; Chen H, et al., Genome Biol. 2019; 20(1):241. Epub 2019 Nov. 20; Pliner H A, et al., Mol Cell. 2018; 71(5):858-71 e8. Epub 2018 Aug. 7). Analysis of cells in the transition stage will provide insights into enhancers and TFs that play a role in the initiation of the T_{H}IFNR cells.

Expression of key TFs predicted to be important in both human and murine T_{H}IFNR cells are determined in the scRNA-seq datasets from the same cell types. Once the expression of key candidate TFs is verified, actual DNA binding are tested by TF ChIP assays, as described (Schmiedel B J, et al., Cell. 2018; 175(6):1701-15 e16. Epub 2018 Nov. 20; Cayford J, et al., J Vis Exp. 2020(162). Epub 2020 Sep. 1; Schmiedel B J, et al., Nat Commun. 2016; 7:13426. Epub 2016 Nov. 17). 2-5 of the top predicted TFs are evaluated using commercial antibodies. CUT and Run assays (Meers M P, et al., Elife. 2019; 8. Epub 2019 Jun. 25) are employed to confirm binding of TF to target enhancer regions.

Functional Analysis The role of the top TF factors are functionally tested using genetically-modified mouse strains (knock-out mice where possible) and it is assessed if the absence of those TF in T cells affect the development and function of T_{H}IFNR cells and impact severity of allergic airway inflammation in mouse models. Where genetically modified mouse strains are not available, TF-specific shRNAmir sequences are expressed from LMPd-Ametrine vectors to knock down TFs of interest (Chen R et al., Immunity. 2014; 41(2):325-38. Epub 2014 Aug. 26) in naive CD4⁺ T cells from DER-1 transgenic mice using protocols that are well-optimized. DER-1 T cells with altered levels of candidate TFs or control cells (scramble shRNAmir) are adoptively transferred into wildtype mice that are subjected to HDM-induced asthma model (as described in 1C). These studies establish if the predicted TFs play a role in the development and function of T_{H}IFNR cells in vivo. Separately, expression of the same TFs are enhanced in CD4⁺ T cells using constitutive expression vectors (e.g., STAT2 retroviruses) with a fluorescent protein marker (Ametrine). This approach has also been used to show that overexpression of certain TFs enhances T_{FH} differentiation (Johnston R J, et al., Science. 2009; 325(5943):1006-10. Epub 2009 Jul. 18; Pedros C, et al., Nature immunology. 2016; 17(7):825-33. Epub 2016 May 3) and CD8⁺ T_{RM} differentiation in vivo (Milner J J, et al., Nature. 2017; 552(7684):253-7. Epub 2017 Dec. 7). Controls—Positive shRNAmir controls target TFs such as STAT2 that would be expected to block IFN signaling; negative shRNAmir controls include non-expressed genes such as CD19. For constitutive overexpression of TF in vector-transduced DER-1 CD4⁺ T cells, empty vector are used as the control. These findings are translated to human T_{H}IFNR cells by testing the effects of knocking down TFs of interest in naive T cells and assessing effects on their differentiation into T_{H}IFNR cells in vitro following TCR and type I IFN signals, as described (Schmiedel B J, et al., Cell. 2018; 175(6):1701-15 e16. Epub 2018 Nov. 20; Schmiedel B J, et al., Nat Commun. 2016; 7:13426. Epub 2016 Nov. 17; Shaw L A, et al., Nat Immunol. 2016; 17(7):834-43. Epub 2016 May 24). Protocols for knocking down candidate TF have been established in primary human T cells (Schmiedel B J, et al., Cell. 2018; 175(6):1701-15 e16. Epub 2018 Nov. 20; Schmiedel B J, et al., Nat Commun. 2016; 7:13426. Epub 2016 Nov. 17).

Without being bound by theory, it is believed that novel TFs predicted by ATAC-Seq analysis play a key role in the differentiation and/or function of T_{H}IFNR cells. If a candidate TF is important, its knockdown should reduce the number of T_{H}IFNR cells generated in vivo and impair their functional activity, i.e., ability to curtain allergic airway inflammation or TRAIL expression. These problems can be mitigated by verifying effects of RNAi using multiple distinct shRNAmirs for each target. Furthermore, overexpression studies provide additional evidence of their importance. If TFs that are specific to human T_{H}IFNR cells only (not conserved in mice) are defined, then functional studies are restricted to studies of human T cell differentiation in vitro.

### Example 3

In EXAMPLE 2, functionally studies evaluate a newly discovered subset of CD4⁺ T cells with immune-regulatory function, called T_{H}IFNR, in the context of allergy and asthma. To achieve this, following describes work as exemplified in EXAMPLE 1, interferon-stimulated response element (ISRE) reporter mice (Uccellini M B, et al., Cell Rep. 2018; 25(10):2784-96 e3. Epub 2018 Dec. 6; Stifter S A, et al., Cell Rep. 2019; 29(11):3539-50 e4. Epub 2019 Dec. 12) is used and the importance of T_{H}IFNR cells is determined in controlling allergic airway inflammation in different murine models of allergy.

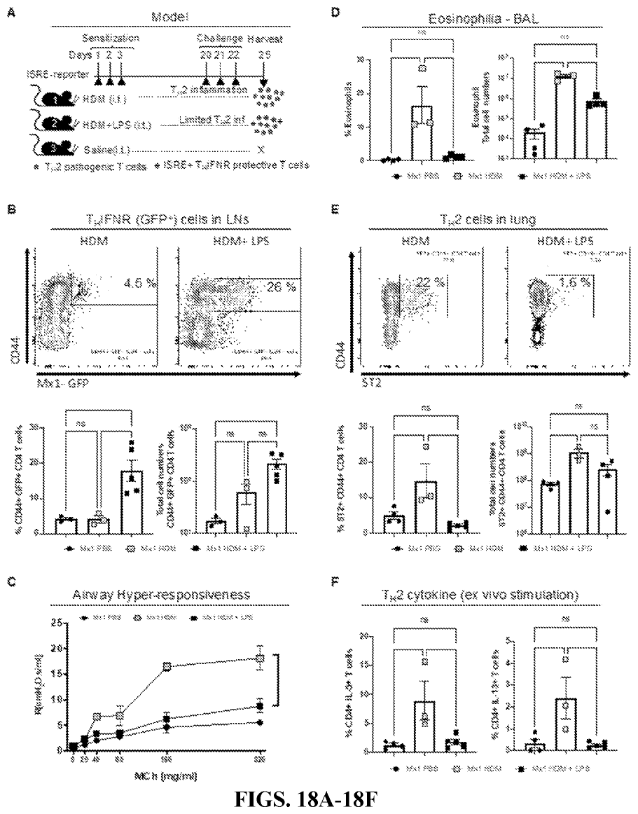

Multiple strains of mouse were acquired: ISRE Mx1-GFP, IFNAR1^{-/-}, STAT2^{-/-}, Lck-CRE, and the ISRE Irgm1-DsRed reporter mice (transfer from Sydney, AU). The ‘asthma-protection model’ was established and it showed that this model generates T_{H}IFNR cells (GFP⁺). Briefly, as described in EXAMPLE 1, mice were administered intratracheally (i.t.) with 50 μg of House Dust Mite (HDM) extract±LPS from Escherichia coli 0111:B4 (Sigma-Aldrich) daily for 1-3 days and challenged intranasally (i.n.) with 50 μg of HDM on days 20-22, and then analyzed on day 25 (FIG. 18A, and FIG. 15). Main outcome results are provided, such as a significant increased frequencies and numbers of T_{H}IFNR (GFP⁺) cells in the lung draining lymph nodes (FIG. 18B) in the HDM/LPS model. Additionally, the ‘asthma-protection’ model suggests a T_{H}2 disease limiting phenotype associated with the generation of the T_{H}IFNR cells. Indeed, in comparison to the standard ‘asthma-inflammation’ model, significate reduction was observed of (i) airway hyperresponsiveness measurements (cmH₂O/mL/s) (FIG. 18C), (ii) airway eosinophilia in BAL (FIG. 18D), (iii) lung CD44⁺ ST2⁺ T_{H}2 cells proportions and counts (FIG. 18E), and (iv) ex vivo T_{H}2 cytokines measurement after PMA stimulation for 6 hr with Brefeldin (2 hr) (FIG. 18F). Altogether, these data support that the development of T_{H}IFNR cells in allergic mice induce a reduction of T_{H}2 inflammatory response hall marks of allergic disease such as AHR, eosinophilia and T_{H}2 cells recruitment, accumulation and activation in the local lung tissue. Accordingly, the role of T_{H}IFNR cells is defined in vivo.

### Exemplified Embodiments of the Disclosure

Embodiment 1. A method of detecting the presence of an antigen-specific T cell characterized with an elevated expression level of an interferon response gene profile in a biological sample, comprising:
a) generating a gene expression profile of at least one T cell from each of a population of CD154-enriched T cells and a population of CD137-enriched T cells obtained from a biological sample by a sequencing method; and
b) analyzing the gene expression profile to detect the presence of an antigen-specific T cell that express an elevated level of an interferon response gene profile in the biological sample.

Embodiment 2. The method of embodiment 1, wherein the interferon response gene profile comprises an elevated expression level of at least one of TNFSF10, CXCL10, IFI6, MX1, ISG15, ISG20, OAS1, OAS3, IFIT1, IFIT3, IFITM1, IFI44L, SAMDL9, or a combination thereof.

Embodiment 3. The method of embodiment 1, wherein the interferon response gene profile comprises an elevated expression level of at least one of TNFSF10, CXCL10, or a combination thereof.

Embodiment 4. The method of embodiment 1, wherein the interferon response gene profile comprises an elevated expression level of at least one of IFI6, MX1, ISG15, ISG20, OAS1, OAS3, IFIT1, IFIT3, IFI44L, SAMDL9, or a combination thereof.

Embodiment 5. The method of embodiment 1, wherein the interferon response gene profile comprises an elevated expression level of TNFSF10.

Embodiment 6. The method of embodiment 1, wherein the interferon response gene profile comprises an elevated expression level of CXCL10.

Embodiment 7. The method of any one of the embodiments 1-6, wherein the antigen-specific T cell is an antigen-specific T_{H}2 cell that express an elevated expression level of an interferon response gene profile or an antigen-specific T regulatory (T_{reg}) cell that express an elevated expression level of an interferon response gene profile.

Embodiment 8. The method of embodiment 7, wherein the antigen-specific T_{H}2 cell comprises an elevated expression level of at least one of TNFSF10, CXCL10, IFI6, MX1, ISG15, ISG20, OAS1, OAS3, IFIT1, IFIT3, IFITM1, IFI44L, or a combination thereof.

Embodiment 9. The method of embodiment 7, wherein the antigen-specific T_{H}2 cell comprises an elevated expression level of at least one of IFI6, MX1, ISG15, ISG20, OAS1, OAS3, IFIT1, IFIT3, IFITM1, IFI44L, or a combination thereof.

Embodiment 10. The method of embodiment 7, wherein the antigen-specific T_{H}2 cell comprises an elevated expression level of at least one of TNFSF10, CXCL10, or a combination thereof.

Embodiment 11. The method of embodiment 7, wherein the antigen-specific T_{H}2 cell comprises an elevated expression level of TNFSF10

Embodiment 12. The method of embodiment 7, wherein the antigen-specific T_{H}2 cell comprises an elevated expression level of CXCL10.

Embodiment 13. The method of embodiment 7, wherein the antigen-specific T_{reg} cell comprises an elevated expression level of at least one of TNFSF10, ISG15, IFI6, MX1, IFIT3, SAMDL9, OAS1, or a combination thereof.

Embodiment 14. The method of embodiment 7, wherein the antigen-specific T_{reg} cell comprises an elevated expression level of TNFSF10.

Embodiment 15. The method of embodiment 7, wherein the antigen-specific T_{reg} cell comprises an elevated expression level of at least one of ISG15, IFI6, MX1, IFIT3, SAMDL9, OAS1, or a combination thereof.

Embodiment 16. The method of any one of the embodiments 1-15, wherein the antigen-specific T cell is a house dust mite (HDM)-reactive T cell.

Embodiment 17. The method of embodiment 16, wherein the HDM-reactive T cell is characterized with an interferon response gene profile comprising an elevated expression level of at least one of TNFSF10, CXCL10, IFI6, MX1, ISG15, ISG20, OAS1, OAS3, IFIT1, IFIT3, IFITM1, IFI44L, SAMDL9, or a combination thereof.

Embodiment 18. The method of embodiment 16 or 17, wherein the HDM-reactive T cell is a HDM-reactive T_{H}2 cell that express an elevated expression level of an interferon response gene profile (T_{H}IFNR) or a HDM-reactive T regulatory (T_{reg}) cell that express an elevated expression level of an interferon response gene profile (T_{reg}IFNR).

Embodiment 19. The method of any one of the embodiments 16-18, wherein the T_{H}IFNR comprises an elevated expression level of at least one of TNFSF10, CXCL10, IFI6, MX1, ISG15, ISG20, OAS1, OAS3, IFIT1, IFIT3, IFITM1, IFI44L, or a combination thereof.

Embodiment 20. The method of any one of the embodiments 16-18, wherein the T_{H}IFNR comprises an elevated expression level of at least one of IFI6, MX1, ISG15, ISG20, OAS1, OAS3, IFIT1, IFIT3, IFITM1, IFI44L, or a combination thereof.

Embodiment 21. The method of any one of the embodiments 16-18, wherein the T_{H}IFNR comprises an elevated expression level of at least one of TNFSF10, CXCL10, or a combination thereof.

Embodiment 22. The method of any one of the embodiments 16-18, wherein the T_{H}IFNR comprises an elevated expression level of TNFSF10.

Embodiment 23. The method of any one of the embodiments 16-18, wherein the T_{H}IFNR comprises an elevated expression level of CXCL10.

Embodiment 24. The method of any one of the embodiments 16-18, wherein the T_{reg}IFNR comprises an elevated expression level of at least one of TNFSF10, ISG15, IFI6, MX1, IFIT3, SAMDL9, OAS1, or a combination thereof.

Embodiment 25. The method of any one of the embodiments 16-18, wherein the T_{reg}IFNR comprises an elevated expression level of TNFSF10.

Embodiment 26. The method of any one of the embodiments 16-18, wherein the T_{reg}IFNR comprises an elevated expression level of at least one of ISG15, IFI6, MX1, IFIT3, SAMDL9, OAS1, or a combination thereof.

Embodiment 27. The method of any one of the embodiments 1-26, wherein the interferon response gene profile further comprises an elevated expression level of at least one of XCL2, SEMATA, CXCR3, FASLG, IFNG, PRF1, KLRG1, XCL1, CD226, NFATC1, or a combination thereof.

Embodiment 28. The method of any one of the embodiments 1-27, wherein the interferon response gene profile comprises an elevated expression level of a gene selected from T_{H}IFNR group of Table 1.

Embodiment 29. The method of any one of the embodiments 1-28, wherein the interferon response gene profile comprises an elevated expression level of a gene selected from T_{H}1 group of Table 1.

Embodiment 30. The method of any one of the embodiments 1-29, wherein the elevated expression level of the interferon response gene profile is compared to a control.

Embodiment 31. The method of embodiment 30, wherein the control is an interferon response gene profile of a CD154 positive T cell that has not been stimulated with HDM.

Embodiment 32. The method of embodiment 30, wherein the control is an interferon response gene profile of a CD137 positive T cell that has not been stimulated with HDM.

Embodiment 33. The method of any one of the embodiments 30-32, wherein the expression level of the interferon response gene profile compared to the control is elevated by about 1-fold, 2-fold, 3-fold, 4-fold, 5-fold, 6-fold, 7-fold, 8-fold, 9-fold, 10-fold, 20-fold, 30-fold, 50-fold, 100-fold, or higher.

Embodiment 34. The method of any one of the embodiments 30-32, wherein the expression level of the interferon response gene profile compared to the control is elevated by about 10%, 20%, 30%, 40%, 50%, 60%, 70%, 80%, 90%, 100%, 200%, 500%, or more.

Embodiment 35. The method of embodiment 1, further comprising harvesting the antigen-specific T cell identified in step c) that express an elevated level of an interferon response gene profile.

Embodiment 36. A method of detecting the presence of an interleukin 9 (IL-9)-expressing antigen-specific T cell in a biological sample, comprising:
a) generating a gene expression profile for at least one T cell from each of a population of CD154-enriched T cells and a population of CD137-enriched T cells obtained from a biological sample by a sequencing method; and
b) analyzing the gene expression profile to detect the presence of an IL-9-expressing antigen-specific T cell in the biological sample.

Embodiment 37. The method of embodiment 36, wherein the analyzing comprises detecting the expression or level of gene IL-5, IL-9, IL1RL1, ZEB2, MAF, MAP3K8, GZMB, MT-ND5, DUSP6, SEC61G, DNAJC3, AHI1, CDK2AP2, FKBP11, FKBP1A, KDELR2, CD109, or SEC61B, or a combination thereof.

Embodiment 38. The method of embodiment 36, wherein the analyzing further comprises detecting the expression or level of gene PPARG, TGFBR3, IL-13, IL-4, IL-31, IL-3, IL-33R, ICOS, IL-21, PLA2G16, GATA3, IL17RB, GADD45G, EFHD2, RAB27A, or RUNX3, or a combination thereof.

Embodiment 39. The method of embodiment 36, wherein the analyzing further comprises detecting the expression or level of gene CD28, BTLA, CTLA-4, PD-1, or a gene encoding a HVEM receptor, or a combination thereof.

Embodiment 40. The method of embodiment 36, wherein the HVEM receptor is encoded by TNFSF14.

Embodiment 41. The method of embodiment 36, wherein the analyzing further comprises detecting the expression or level of gene NFKBID, NIPAI, FOSL2, NEDD9, BCL2A1, BIRC3, DUSP4/MKP-2, or CFLAR, or a combination thereof.

Embodiment 42. The method of embodiment 36, wherein the analyzing further comprises detecting the expression or level of a gene selected from the T_{H}2 group of Table 1.

Embodiment 43. The method of any one of the embodiments 36-42, wherein the IL-9-expressing antigen-specific T cell is an IL-9-expressing T_{H}2 cell.

Embodiment 44. The method of embodiment 43, wherein the IL-9-expressing antigen-specific T cell is a IL-9-expressing house dust mite (HDM)-reactive T cell.

Embodiment 45. The method of any one of the embodiments 36-44, wherein the IL-9-expressing HDM-reactive T cell is an IL-9-expressing HDM-reactive T_{H}2 cell.

Embodiment 46. The method of any one of the embodiments 1-45, wherein the biological sample is processed by a flow cytometry method.

Embodiment 47. The method of any one of the embodiments 1-46, wherein the biological sample is contacted with a plurality of beads labeled with CD154 antibodies, optionally biotinylated CD154 antibodies, and processed to generate the population of CD154-enriched T cells.

Embodiment 48. The method of any one of the embodiments 1-47, wherein the biological sample is contacted with a plurality of beads labeled with CD137 antibodies, optionally biotinylated CD137 antibodies, and processed to generate the population of CD137-enriched T cells.

Embodiment 49. The method of embodiment 47 or 48, wherein the plurality of beads are magnetized beads.

Embodiment 50. The method of any one of the embodiments 1-49, wherein the biological sample is a peripheral blood mononuclear cell (PBMC) sample.

Embodiment 51. The method of any one of the embodiments 1-50, wherein the sequencing method comprises an amplification method.

Embodiment 52. The method of any one of the embodiments 1-51, wherein the sequencing method comprises generating a plurality of barcoded RNAs.

Embodiment 53. The method of any one of the embodiments 1-52, wherein the sequencing method comprises performing a pairwise differential gene expression analysis, optionally a pairwise single-cell differential gene expression analysis.

Embodiment 54. The method of any one of the embodiments 1-53, wherein the biological sample is obtained from a subject.

Embodiment 55. The method of embodiment 54, wherein the subject is suspected of suffering from an inflammation of the skin, airway mucosa, or a combination thereof, optionally due to HDM.

Embodiment 56. The method of embodiment 54 or 55, wherein the subject is suspected of suffering from atopic dermatitis, allergic rhinitis, allergic asthma, allergic conjunctivitis, or a combination thereof.

Embodiment 57. The method of any one of the embodiments 1-56, wherein the biological sample is further stimulated with HDM, optionally a HDM peptide, prior to processing the biological sample to generate the population of CD154-enriched T cells and the population of CD137-enriched T cells.

Embodiment 58. The method of any one of the embodiments 1-57, wherein the biological sample is cultured post stimulation for about 2, 3, 4, or 5 days prior to processing the biological sample.

Embodiment 59. A method of treating an allergy in a subject in need thereof or selecting a subject for treatment of an allergy, comprising:
detecting the presence of an IL-9-expressing antigen-specific T cell in a biological sample obtained from the subject to identify the subject has expressing the IL-9-expressing antigen-specific T cell; and
administering an anti-allergy therapy to the subject expressing the IL-9-expressing antigen-specific T cell.

Embodiment 60. The method of embodiment 59, wherein the IL-9-expressing antigen-specific T cell is detected by the method of any one of embodiments 36-58.

Embodiment 61. The method of embodiment 59 or embodiment 60, wherein the anti-allergy therapy comprises administering a therapeutic agent selected from an antihistamine, a corticosteroid, a mast cell stabilizer, and a leukotriene modifier.

Embodiment 62. The method of embodiment 61, wherein the antihistamine comprises azelastine, cetirizine, chlorpheniramine, diphenhydramine, fexofenadine, levocetirizine, loratadine, olopatadine, promethazine, or triprolidine.

Embodiment 63. The method of embodiment 61, wherein the corticosteroid comprises bethamethasone, ciclesonide, dexamethasone, fluticasone, methylprednisolone, mometasone, prednisone, prednisolone, or triamcinolone.

Embodiment 64. The method of embodiment 61, wherein the mast cell stabilizer comprises cromolyn sodium.

Embodiment 65. The method of embodiment 61, wherein the leukotriene modifier comprises montelukast.

Embodiment 66. The method of embodiment 59 or embodiment 60, wherein the anti-allergy therapy comprises administering to the subject a specific immunotherapy (SIT) treatment or regimen.

Embodiment 67. The method of embodiment 66, wherein the specific immunotherapy comprises sub-cutaneous immunotherapy (SCIT) or sublingual immunotherapy (SLIT).

Embodiment 68. The method of any one of the embodiments 59-67, wherein the allergy comprises an inflammation of the skin, airway mucosa, or a combination thereof.

Embodiment 69. The method of any one of the embodiments 59-68, wherein the allergy is atopic dermatitis, allergic rhinitis, allergic asthma, allergic conjunctivitis, or a combination thereof.

Embodiment 70. The method of any one of the embodiments 59-69, wherein the allergy is a HDM-induced allergy.

Embodiment 71. The method of any one of the embodiments 59-70, wherein the HDM-induced allergy comprises an inflammation of the skin, airway mucosa, or a combination thereof.

Embodiment 72. The method of any one of the embodiments 59-71, wherein the HDM-induced allergy is atopic dermatitis, allergic rhinitis, allergic asthma, allergic conjunctivitis, or a combination thereof.

Embodiment 73. A composition comprising a plurality of antigen-specific T cells detectable using the method of any one of embodiments 1-35 or 46-58; and optionally comprising a carrier, excipient, stabilizer or preservative.

Embodiment 74. A method of treating an allergy in a subject in need thereof comprising administering to the subject the composition of embodiment 73.

Embodiment 75. The method of embodiment 74, wherein the plurality of antigen-specific T cells are allogenic to the subject.

Embodiment 76. The method of embodiment 74, wherein the plurality of antigen-specific T cells are autologous to the subject.

Embodiment 77. The method of any one of the embodiments 74-76, wherein the subject has an inflammation of the skin, airway mucosa, or a combination thereof, optionally due to HDM.

Embodiment 78. The method of any one of the embodiments 74-76, wherein the subject has atopic dermatitis, allergic rhinitis, allergic asthma, allergic conjunctivitis, or a combination thereof, optionally due to HDM.

Embodiment 79. The method of any one of the embodiments 74-78, wherein the plurality of antigen-specific T cells are a plurality of HDM-reactive T cells, optionally allogenic or autologous to the subject.

Embodiment 80. The method of any one of the embodiments 74-79, wherein the method further comprises administering to the subject a therapeutic agent, optionally selected from an antihistamine, a corticosteroid, a mast cell stabilizer, and a leukotriene modifier.

Embodiment 81. A method of generating a TRAIL-expressing T cell, comprising:
incubating a biological sample comprising a plurality of CD4+ T cells with an isolated and recombinant TRAIL protein for at least 10 minutes to generate a population of stimulated T cells; and
separating the population of stimulated T cells by a flow cytometry method to isolate a TRAIL-expressing T cell.

Embodiment 82. The method of embodiment 81, wherein the isolated and recombinant TRAIL protein is a full-length protein or a functional fragment thereof.

Embodiment 83. The method of embodiment 81, wherein the isolated and recombinant TRAIL protein is a wild-type protein or a variant thereof.

Embodiment 84. The method of any one of the embodiments 81-83, wherein the isolated and recombinant TRAIL protein is incubated with the biological sample for at least 30 minutes, 1 hour, 2 hours, 3 hours, 4 hours, 5 hours, 6 hours, or more.

Embodiment 85. The method of any one of the embodiments 81-84, wherein the TRAIL-expressing T cell is a TRAIL-expressing T_{H}2 cell or a TRAIL-expressing T_{reg} cell.

Embodiment 86. The method of embodiment 81, further comprising administering the isolated TRAIL-expressing T cell to a subject in need thereof.

Embodiment 87. The method of any one of the embodiments 81-86, wherein the subject has an allergic response, optionally to HDM.

Embodiment 88. The method of any one of the embodiments 81-87, wherein the subject has an inflammation of the skin, airway mucosa, or a combination thereof, optionally due to HDM.

Embodiment 89. The method of any one of the embodiments 81-88, wherein the subject has atopic dermatitis, allergic rhinitis, allergic asthma, allergic conjunctivitis, or a combination thereof, optionally due to HDM.

Embodiment 90. The method of any one of the embodiments 81-89, wherein the isolated TRAIL-expressing T cell is allogenic to the subject.

Embodiment 91. The method of any one of the embodiments 81-89, wherein the isolated TRAIL-expressing T cell is autologous to the subject.

Embodiment 92. The method of any preceding embodiments, wherein the subject is a human.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scope of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control. Throughout this disclosure, various publication are referenced by a citation, the full bibliographic citation for each are provided immediately preceding the claims.

What is claimed is:

1. A method of detecting the presence of an antigen-specific T cell characterized with an elevated expression level of an interferon response gene profile in a biological sample, comprising:
c) generating a gene expression profile of at least one T cell from each of a population of CD154-enriched T cells, a population of CD137-enriched T cells, or a population of CD4-enriched T cells obtained from the biological sample by a sequencing method; and
d) analyzing the gene expression profile to detect the presence of the antigen-specific T cell that expresses the elevated level of the interferon response gene profile in the biological sample.

2. The method of claim 1, wherein the interferon response gene profile comprises an elevated expression level of at least one of TNFSF10, CXCL10, IFI6, MX1, ISG15, ISG20, OAS1, OAS3, IFIT1, IFIT3, IFITM1, IFI44L, SAMDL9, or a combination thereof.

3. The method of claim 1, wherein the interferon response gene profile comprises an elevated expression level of at least one of TNFSF10, CXCL10, or a combination thereof.

4. The method of claim 1, wherein the interferon response gene profile comprises an elevated expression level of at least one of IFI6, MX1, ISG15, ISG20, OAS1, OAS3, IFIT1, IFIT3, IFITM1, IFI44L, SAMDL9, or a combination thereof.

5. The method of claim 1, wherein the interferon response gene profile comprises an elevated expression level of TNFSF10.

6. The method of claim 1, wherein the interferon response gene profile comprises an elevated expression level of CXCL10.

7. The method of any one of the claims 1-6, wherein the antigen-specific T cell is an antigen-specific T_{H} cell that express an elevated expression level of an interferon response gene profile or an antigen-specific T regulatory (T_{reg}) cell that express an elevated expression level of an interferon response gene profile.

8. The method of claim 7, wherein the antigen-specific T_{H} cell comprises an elevated expression level of at least one of TNFSF10, CXCL10, IFI6, MX1, ISG15, ISG20, OAS1, OAS3, IFIT1, IFIT3, IFITM1, IFI44L, or a combination thereof.

9. The method of claim 7, wherein the antigen-specific T_{H} cell comprises an elevated expression level of at least one of IFI6, MX1, ISG15, ISG20, OAS1, OAS3, IFIT1, IFIT3, IFITM1, IFI44L, or a combination thereof.

10. The method of claim 7, wherein the antigen-specific T_{H} cell comprises an elevated expression level of at least one of TNFSF10, CXCL10, or a combination thereof.

11. The method of claim 7, wherein the antigen-specific T_{H} cell comprises an elevated expression level of TNFSF10.

12. The method of claim 7, wherein the antigen-specific T_{H} cell comprises an elevated expression level of CXCL10.

13. The method of claim 7, wherein the antigen-specific T_{reg} cell comprises an elevated expression level of at least one of TNFSF10, ISG15, IFI6, MX1, IFIT3, SAMDL9, OAS1, or a combination thereof.

14. The method of claim 7, wherein the antigen-specific T_{reg} cell comprises an elevated expression level of TNFSF10.

15. The method of claim 7, wherein the antigen-specific T_{reg} cell comprises an elevated expression level of at least one of ISG15, IFI6, MX1, IFIT3, SAMDL9, OAS1, or a combination thereof.

16. The method of any one of the claims 1-15, wherein the antigen-specific T cell is a house dust mite (HDM)-reactive T cell.

17. The method of claim 16, wherein the HDM-reactive T cell is characterized with an interferon response gene profile comprising an elevated expression level of at least one of TNFSF10, CXCL10, IFI6, MX1, ISG15, ISG20, OAS1, OAS3, IFIT1, IFIT3, IFIT111, IFI44L, SAMDL9, or a combination thereof.

18. The method of claim 16 or 17, wherein the HDM-reactive T cell is a HDM-reactive T_{H} cell that express an elevated expression level of an interferon response gene profile (T_{H}IFNR) or a HDM-reactive T regulatory (T_{reg}) cell that express an elevated expression level of an interferon response gene profile (T_{reg}IFNR).

19. The method of any one of the claims 16-18, wherein the T_{H}IFNR comprises an elevated expression level of at least one of TNFSF10, CXCL10, IFI6, MX1, ISG15, ISG20, OAS1, OAS3, IFIT1, IFIT3, IFITM1, IFI44L, or a combination thereof.

20. The method of any one of the claims 16-18, wherein the T_{H}IFNR comprises an elevated expression level of at least one of IFI6, MX1, ISG15, ISG20, OAS1, OAS3, IFIT1, IFIT3, IFITM1, IFI44L, or a combination thereof.

21. The method of any one of the claims 16-18, wherein the T_{H}IFNR comprises an elevated expression level of at least one of TNFSF10, CXCL10, or a combination thereof.

22. The method of any one of the claims 16-18, wherein the T_{H}IFNR comprises an elevated expression level of TNFSF10.

23. The method of any one of the claims 16-18, wherein the T_{H}IFNR comprises an elevated expression level of CXCL10.

24. The method of any one of the claims 16-18, wherein the T_{reg}IFNR comprises an elevated expression level of at least one of TNFSF10, ISG15, IFI6, MX1, IFIT3, SAMDL9, OAS1, or a combination thereof.

25. The method of any one of the claims 16-18, wherein the T_{reg}IFNR comprises an elevated expression level of TNFSF10.

26. The method of any one of the claims 16-18, wherein the T_{reg}IFNR comprises an elevated expression level of at least one of ISG15, IFI6, MX1, IFIT3, SAMDL9, OAS1, or a combination thereof.

27. The method of any one of the claims 1-26, wherein the interferon response gene profile further comprises an elevated expression level of at least one of XCL2, SEMATA, CXCR3, FASLG, IFNG, PRF1, KLRG1, XCL1, CD226, NFATC1, or a combination thereof.

28. The method of any one of the claims 1-27, wherein the interferon response gene profile comprises an elevated expression level of a gene selected from T_{H}IFNR group of Table 1.

29. The method of any one of the claims 1-28, wherein the interferon response gene profile comprises an elevated expression level of a gene selected from T_{H}1 group of Table 1.

30. The method of any one of the claims 1-29, wherein the elevated expression level of the interferon response gene profile is compared to a control.

31. The method of claim 30, wherein the control is an interferon response gene profile of a CD154 positive T cell that has not been stimulated with an antigen specifically recognized and bound by the antigen-specific T cell, optionally HDM.

32. The method of claim 30, wherein the control is an interferon response gene profile of a CD137 positive T cell that has not been stimulated with the antigen, optionally HDM.

33. The method of claim 30, wherein the control is an interferon response gene profile of a CD4 positive T cell that has not been stimulated with the antigen, optionally HDM.

34. The method of claim 30, wherein the control is the expression level of the interferon response gene profile of one or more of a T_{H}1 cell, a T_{H}2 cell, a T_{H}17, a T_{H}ACT1, a T_{H}ACT2, or a T_{H}ACT3, TREGACT1, TREGACT2, or any combination thereof.

35. The method of any one of the claims 30-34, wherein the expression level of the interferon response gene profile compared to the control is elevated by about 1-fold, 2-fold, 3-fold, 4-fold, 5-fold, 6-fold, 7-fold, 8-fold, 9-fold, 10-fold, 20-fold, 30-fold, 50-fold, 100-fold, or higher.

36. The method of any one of the claims 30-34, wherein the expression level of the interferon response gene profile compared to the control is elevated by about 10%, 20%, 30%, 40%, 50%, 60%, 70%, 80%, 90%, 100%, 200%, 500%, or more.

37. The method of any one of the claims 1-36, further comprising enriching or isolating or enriching and isolating the antigen-specific T cell identified in step b) that expresses an elevated level of an interferon response gene profile.

38. The method of any one of the claims 1-37, wherein the T cells in the biological sample are enriched by a flow cytometry method.

39. The method of any one of the claims 1-38, wherein the population of CD154-enriched T cells are enriched by contacting the biological sample with a plurality of beads labeled with CD154 antibodies or biotinylated CD154 antibodies.

40. The method of any one of the claims 1-39, wherein the population of CD137-enriched T cells are enriched by contacting the biological sample with a plurality of beads labeled with CD137 antibodies or biotinylated CD137 antibodies.

41. The method of any one of the claims 1-40, wherein the population of CD4-enriched T cells are enriched by contacting the biological sample with a plurality of beads labeled with CD4 antibodies or biotinylated CD4 antibodies.

42. The method of any one of the claims 39-41, wherein the plurality of beads are magnetized beads.

43. The method of any one of the claims 1-42, wherein the biological sample is a peripheral blood mononuclear cell (PBMC) sample.

44. The method of any one of claims 1-42, wherein the biological sample is selected from a broncheoalveolar lavage fluid sample, a lung draining lymph node sample, or a lung biopsy.

45. The method of any one of the claims 1-44, wherein the sequencing method comprises an amplification method.

46. The method of any one of the claims 1-45, wherein the sequencing method comprises generating a plurality of barcoded RNAs.

47. The method of any one of the claims 1-46, wherein the sequencing method comprises performing a pairwise differential gene expression analysis, optionally a pairwise single-cell differential gene expression analysis.

48. The method of any one of the claims 1-47, wherein the biological sample is obtained from a subject.

49. The method of claim 48, wherein the subject is suspected of suffering from an inflammation of the skin, airway mucosa, or a combination thereof, optionally due to HDM.

50. The method of claim 48 or 49, wherein the subject is suspected of suffering from atopic dermatitis, allergic rhinitis, allergic asthma, allergic conjunctivitis, or a combination thereof.

51. The method of claim 48, wherein the subject is suspected of suffering from a pathogenic infection, optionally a viral infection.

52. The method of claim 51, wherein the viral infection is induced by a coronavirus, optionally an alpha-type coronavirus or a beta-type coronavirus, further optionally 229E, NL63, OC43, HKU1, MERS-CoV, SARS-CoV, or SARS-CoV-2.

53. The method of claim 51, wherein the viral infection is induced by an influenza virus, optionally an influenza A virus.

54. The method of claim 51, wherein the viral infection is induced by cytomegalovirus, Epstein-Barr virus, variola virus, Ebola, dengue, Measles virus, mumps virus, or rubella virus.

55. The method of claim 51, wherein the pathogenic infection is induced by a bacterium, a fungus, a protozoan, or a parasite.

56. The method of claim 48, wherein the subject lacks an inflammation of the skin, airway mucosa, or a combination thereof, optionally due to HDM.

57. The method of claim 48, wherein the subject is a healthy subject.

58. The method of claim 48, wherein the subject is free of an allergy.

59. The method of any one of the claims 1-58, wherein the biological sample is further stimulated with an antigen specifically recognized and bound by the antigen-specific T cells, optionally HDM, further optionally a HDM peptide, prior to processing the biological sample to generate the population of CD154-enriched T cells, the population of CD137-enriched T cells, or the population of CD4-enriched T cells.

60. The method of any one of the claims 1-59, wherein the biological sample is cultured post stimulation for about 2, 3, 4, or 5 days prior to processing the biological sample.

61. A composition comprising a plurality of antigen-specific T cells detectable using the method of any one of the claims 1-60 or the T cells enriched or isolated using the method of any one of the claims 37-60; and optionally further comprising a carrier, excipient, stabilizer or preservative.

62. A method of generating a TRAIL-expressing T cell, comprising:
incubating a biological sample comprising a plurality of CD4+ T cells with a TCR stimulator, optionally selected from an antigen, an anti-CD3 antibody, an anti-CD28 antibody, or any combination thereof, for at least 10 minutes to generate a population of stimulated T cells; and
isolating or enriched or both isolating and enriching TRAIL-expressing T cells from the stimulated T cells.

63. The method of claim 62, wherein the incubation with the biological sample is at least 30 minutes, 1 hour, 2 hours, 3 hours, 4 hours, 5 hours, 6 hours, or longer.

64. A method comprising expanding or isolating or both expanding and isolating a plurality of CD4+ T cells that express an elevated level of an interferon response gene profile.

65. The method of claim 64, further comprising stimulating the plurality of CD4+ T cells by contacting with a TCR stimulator, optionally selected from an anti-CD3 antibody or an anti-CD28 antibody or both.

66. The method of any one of the claims 62-65, wherein the isolated or enriched T cells comprise a TRAIL-expressing T_{H} cell or a TRAIL-expressing T_{reg} cell.

67. The method of any one of the claims 62-66, wherein the isolated or enriched T cells comprise a T_{H}IFNR cell comprising an elevated expression level of at least one of TNFSF10, CXCL10, IFI6, MX1, ISG15, ISG20, OAS1, OAS3, IFIT1, IFIT3, IFITM1, IFI44L, or a combination thereof.

68. The method of any one of the claims 62-67, wherein the isolated or enriched T cells comprise a T_{H}IFNR cell comprising an elevated expression level of at least one of IFI6, MX1, ISG15, ISG20, OAS1, OAS3, IFIT1, IFIT3, IFITM1, IFI44L, or a combination thereof.

69. The method of any one of the claims 62-68, wherein the isolated or enriched T cells comprise a T_{H}IFNR cell comprising an elevated expression level of at least one of TNFSF10, CXCL10, or a combination thereof.

70. The method of any one of the claims 62-69, wherein the isolated or enriched T cells comprise a T_{H}IFNR cell comprising an elevated expression level of TNFSF10.

71. The method of any one of the claims 62-70, wherein the isolated or enriched T cells comprise a T_{H}IFNR cell comprising an elevated expression level of CXCL10.

72. The method of any one of the claims 62-71, wherein the isolated or enriched T cells comprise a T_{reg}IFNR cell comprising an elevated expression level of at least one of TNFSF10, ISG15, IFI6, MX1, IFIT3, SAMDL9, OAS1, or a combination thereof.

73. The method of any one of the claims 62-72, wherein the isolated or enriched T cells comprise a T_{reg}IFNR cell comprising an elevated expression level of TNFSF10.

74. The method of any one of the claims 62-73, wherein the isolated or enriched T cells comprise a T_{reg}IFNR cell comprising an elevated expression level of at least one of ISG15, IFI6, MX1, IFIT3, SAMDL9, OAS1, or a combination thereof.

75. The method of any one of the claims 64-74, wherein the interferon response gene profile further comprises an elevated expression level of at least one of XCL2, SEMATA, CXCR3, FASLG, IFNG, PRF1, KLRG1, XCL1, CD226, NFATC1, or a combination thereof.

76. The method of any one of the claims 64-75, wherein the interferon response gene profile comprises an elevated expression level of a gene selected from T_{H}IFNR group of Table 1.

77. A composition comprising T cells isolated or enriched using the method of any one of the claims 62-76 and optionally further comprising a carrier, excipient, stabilizer or preservative.

78. A method of treating an allergy in a subject in need thereof comprising administering to the subject the composition of claim 61 or claim 77.

79. The method of claim 78, wherein the subject has an inflammation of the skin, airway mucosa, or a combination thereof, optionally due to HDM.

80. The method of claim 78 or 79, wherein the subject has atopic dermatitis, allergic rhinitis, allergic asthma, allergic conjunctivitis, or a combination thereof.

81. The method of any one of the claims 78-80, wherein the plurality of T cells are a plurality of HDM-reactive T cells, optionally allogenic or autologous to the subject.

82. The method of any one of the claims 78-81, wherein the method further comprises administering to the subject a therapeutic agent, optionally selected from an antihistamine, a corticosteroid, a mast cell stabilizer, or a leukotriene modifier.

83. A method of treating an infection in a subject in need thereof comprising administering to the subject the composition of claim 61 or claim 77.

84. The method of claim 83, wherein the subject has a pathogenic infection, optionally a viral infection.

85. The method of claim 84, wherein the viral infection is induced by a coronavirus, optionally an alpha-type coronavirus or a beta-type coronavirus, further optionally 229E, NL63, OC43, HKU1, MERS-CoV, SARS-CoV, or SARS-CoV-2.

86. The method of claim 84, wherein the viral infection is induced by an influenza virus, optionally an influenza A virus.

87. The method of claim 84, wherein the viral infection is induced by cytomegalovirus, Epstein-Barr virus, variola virus, Ebola, dengue, Measles virus, mumps virus, or rubella virus.

88. The method of claim 84, wherein the pathogenic infection is induced by a bacterium, a fungus, a protozoan, or a parasite.

89. The method of any one of the claims 83-88, wherein the method further comprises administering to the subject a therapeutic agent suitable for treating the infection, optionally an anti-viral therapeutic agent or an antibiotic.

90. A method of treating an inflammation in a subject in need thereof comprising administering to the subject the composition of claim 61 or claim 77.

91. The method of claim 90, wherein the inflammation is in the subject's lung.

92. The method of claim 90 or 91, wherein the inflammation is induced by an allergy or an infection.

93. The method of any one of the claims 90-92, wherein the method further comprises administering to the subject a therapeutic agent suitable for treating the inflammation, optionally selected from an antihistamine, a corticosteroid, a mast cell stabilizer, or a leukotriene modifier.

94. The method of any one of the claims 78-93, wherein the plurality of the T cells are allogenic to the subject.

95. The method of any one of the claims 78-93, wherein the plurality of the T cells are autologous to the subject.

96. The method of any preceding claims, wherein the subject is a human.

97. The composition of claim 61 or 77, wherein the T cells are engineered to express a chimeric antigen receptor (CAR).

98. The composition of claim 61 or 77, wherein the T cells express a T cell receptor (TCR).

99. The composition of claim 97 or 98, wherein the CAR or the TCR or both specifically recognizes and binds an antigen of a pathogen or a cancer.

100. The composition of claim 99, wherein the pathogen is selected from a virus, a bacterium, a fungus, a protozoan, or a parasite.

101. The composition of claim 100, wherein the virus is a coronavirus, optionally an alpha-type coronavirus or a beta-type coronavirus, further optionally 229E, NL63, OC43, HKU1, MERS-CoV, SARS-CoV, or SARS-CoV-2.

102. The composition of claim 100, wherein the virus is an influenza virus, optionally an influenza A virus.

103. The composition of claim 100, wherein the virus is selected from cytomegalovirus, Epstein-Barr virus, variola virus, Ebola, dengue, Measles virus, mumps virus, or rubella virus.

104. A method of treating a subject in need thereof, comprising administering the composition of any one of the claims 98-103 to the subject.

105. The method of claim 104, wherein the subject is infected or is suspected of being infected by a pathogen, and wherein the CAR or the TCR or both specifically recognizes and binds an antigen of the pathogen.

106. The method of claim 105, further comprising administering a therapeutic agent suitable for treating the infection.

107. The method of claim 104, wherein the subject has or is suspected of having a cancer, and wherein the CAR or the TCR or both specifically recognizes and binds an antigen of the cancer.

108. The method of claim 107, further comprising applying an anti-cancer therapy to the subject, optionally selected from an ablation therapy, a radiation therapy, a chemotherapy, an immunotherapy, or a targeted therapy.

109. The method of any one of claims 104 to 108, whereby the method does not induce an inflammation in the subject.
